(19) United States
(12) Patent Application Publication
(10) Pub. No.: US 2026/0071277 A1
(43) Pub. Date: Mar. 12, 2026

(54) COMPOSITIONS AND METHODS FOR DETECTING OROPHARYNGEAL CANCER

(71) Applicant: Mayo Foundation for Medical Education and Research, Rochester, MN (US)

(72) Inventors: William R. Taylor, Lake City, MN (US); John B. Kisiel, Rochester, MN (US); Douglas W. Mahoney, Rochester, MN (US)

(21) Appl. No.: 18/705,695

(22) PCT Filed: Nov. 4, 2022
(86) PCT No.: PCT/US2022/079270

§ 371 Date: Apr. 29, 2024

Related U.S. Application Data
(60) Provisional application No. 63/276,058, filed on Nov. 5, 2021.

(51) Int. Cl. C12Q 1/6886

(52) U.S. Cl. CPC C12Q 1/6886; C12Q 2600/154

(57) ABSTRACT

The present disclosure relates to detecting one or more types of oropharyngeal cancer in a biological sample from a subject. In particular, the present disclosure provides compositions and methods for detecting the presence or absence of one or more types of oropharyngeal cancer (e.g., HPV⁺ oropharyngeal squamous cell cancer) in a biological sample from a subject having or suspected of having an oropharyngeal cancer.

### CROSS REFERENCE TO RELATED APPLICATIONS

This application claims priority to and the benefit of U.S. Provisional Patent Application Ser. No. 63/276,058 filed Nov. 5, 2021, which is incorporated herein by reference in its entirety and for all purposes.

### SEQUENCE LISTING

The text of the computer readable sequence listing filed herewith, titled “40013_601_SequenceListing”, created Nov. 4, 2022, having a file size of 154,000 bytes, is hereby incorporated by reference in its entirety.

### FIELD

The present disclosure relates to detecting one or more types of oropharyngeal cancer in a biological sample from a subject. In particular, the present disclosure provides compositions and methods for detecting the presence or absence of one or more types of oropharyngeal cancer (e.g., HPV⁺ oropharyngeal squamous cell cancer) in a biological sample from a subject having or suspected of having an oropharyngeal cancer.

### BACKGROUND

Oropharyngeal (or head and neck) cancers account for 3% of the cancers diagnosed each year in the US and are projected to kill approximately 11,000 people in 2021. While the incidence of HPV-cancers are relatively constant, the HPV+ cancer subtype incidence is increasing. There is an urgent need for a non-invasive molecular test to screen patients at increased risk for oropharyngeal cancer as it likely would decrease the burden of this disease and save lives. The various embodiments disclosed herein address this need.

### SUMMARY

Embodiments of the present disclosure provide methods, compositions, and systems for screening one or more types of oropharyngeal cancer from a biological sample. In accordance with these embodiments, the present disclosure includes, but is not limited to, methods and compositions for detecting the presence of one or more types or subtypes of oropharyngeal cancer from a biological sample. In some embodiments, the biological sample is a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample. In some embodiments, the tissue sample is an oropharyngeal tissue sample comprising one or more of soft palate cells or tissues, throat cells or tissues, tongue cells or tissues, and tonsil cells or tissues. In some embodiments, the tissue sample is an HPV(+) tissue sample. In some embodiments, the subject is a human.

As described further herein, embodiments of the present disclosure include novel differentially methylated regions (DMRs), each individually capable of distinguishing an oropharyngeal cancer (e.g., HPV⁺ oropharyngeal squamous cell cancer (HPV⁺ OPSCC)) from a control sample (e.g., benign tissue, including but not limited to, oropharyngeal tissue, cervical tissue, tonsil tissue, a buffy coat sample, and a saliva sample). In accordance with these embodiments, the novel DMR(s) is from a gene selected from ABCB1, ARHGAP12, ASCL1, Clorf114, EMX1, GRIN2D, LOC645323, MAX.chr6.58147682-58147771, MAX.chr9.36739811-36739868, NEUROG3, NID2, TBX15, TMEM200C, TSPYL5, TTYH1, VWC2, ZNF610, ZNF69, ZNF773, ZNF781, ALX4, ATP10A, C1QL3, CA8, CACNA1A, CACNG8, CALCA, CCNA1, CLIC6, CLSTN2, CR1, CTNND2, DAB1, DGKG, DOK1, DOK6, DPP4, DUXA, ELMO1, EMBP1, EPDR1, FGF12, FLJ43390, FMN2, FOXB2, FOXD4, FREM3, GALR1, GDF6, GFRA1, GRIK3, HOXB3, HOXB4, HPSE2, LDLRAD2, LHX2, LOC100131366, LOC345643, LOC386758, LOC648809, LOC728392, MAML3, MAPRE2, MAX.chr1.226288154-226288189, MAX.chr1.2375078-2375126, MAX.chr1.241587339-241587784, MAX.chr1.50798781-50799423, MAX.chr10.22765150-22765477, MAX.chr10.23462342-23462436, MAX.chr11.14926602-14927044, MAX.chr11.58903531-58903592, MAX.chr13.28527984-28528214, MAX.chr13.29106641-29107037, MAX.chr14.100784488-100784782, MAX.chr16.3221176-3221223, MAX.chr16.3222040-3222098, MAX.chr16.71460171-71460282, MAX.chr19.11805263-11805639, MAX.chr19.16394457-16394646, MAX.chr19.21657626-21657769, MAX.chr19.22034646-22034887, MAX.chr19.23299989-23300156, MAX.chr19.30713427-30713588, MAX.chr19.30716926-30717074, MAX.chr19.30718373-30719719, MAX.chr2.118981724-118982174, MAX.chr2.127783107-127783403, MAX.chr2.173099712-173099791, MAX.chr2.66808635-66808731, MAX.chr22.50064113-50064259, MAX.chr3.137489884-137490061, MAX.chr5.138923141-138923219, MAX.chr5.42995180-42995535, MAX.chr6.38683091-38683226, MAX.chr7.121952014-121952084, MAX.chr7.155166980-155167310, MAX.chr8.99986792-99986864, MAX.chr9.79627078-79627116, MAX.chr9.79638034-79638077, MAX.chr9.98789824-98789847, MDFI, MECOM, MED12L, MIR129-2, MIR196A1, NELL1, NPY, ONECUT2, OPCML, PARP15, PDGFD, PEX5L, PRR15, SEMA6A, SFMBT2, SGIP1, SIM2, SLC35F3, SLCO4C1, SORCS3, ST6GALNAC5, ST8SIA5, SV2C, TACC2, TFAP2E, TLX2, TLX3, TRH, TRIM58, VAV3, VSTM2B, WDR17, ZNF254, ZNF43, ZNF486, ZNF491, ZNF518B, ZNF542, ZNF625, ZNF665, ZNF671, ZNF763, ZNF844, AGRN, ANKRD35, ARHGAP27, ARHGAP30, BCL2L11, BIN2, C10orf114, C4orf31, C6orf132, C6orf186, CCDC88B, CRHBP, DAPK1, DNMT3A, DPP10, FAM19A2, FLJ45983, FOSL1, FOXB1, GREM1, HMHA1, HOXA9, IFFO1, INPP4B, ITGB2, ITGB4, ITPKB, KCNIP2, KLHDC7B, LAT, LHX6, LIMK1, LOC100128239, LOC100192379, LOC646278, MAP2K2, MAX.chr1.210426156-210426257, MAX.chr1.84326495-84326656, MAX.chr10.119312785-119312882, MAX.chr15.67326025-67326060, MAX.chr16.54316401-54316453, MAX.chr16.85482306-85482494, MAX.chr17.74994454-74994572, MAX.chr17.76339840-76339972, MAX.chr2.7571082-7571136, MAX.chr21.45577347-45577679, MAX.chr3.14852538-14852568, MAX.chr3.187676564-187676668, MAX.chr4.174430662-174430793, MAX.chr5.177411809-177411836, MAX.chr6.45631561-45631625, MAX.chr7.25892382-25892451, MAX.chr7.402563-402641, MAX.chr7.64349554-64349606, MAX.chr8.142046239-142046398, MAX.chr8.145900842-145901246, MAX.chr9.126101804-126101848, MAX.chr9.126978999-126979182, MAX.chr9.36458633-36458725, MAX.chr9.87905315-87905326, MBP, MENG, MT1A, MT1IP, NCOR2, NFATC1, NKX3-2, NRN1, OLIG1, PALLD, PAPLN, PDLIM2, PKN1, PRDM14, PRKG1, PRMT7, PTGER2, PTK2B, RAD52, RBM38, RHOF, RNF220, RTN4RL1, RXRA, SDCCAG8, SHROOM1, SKI, SLC12A8, SLC25A47, SPEG, SUCLG2, TBC1D10C, TMEM132E, VIPR2, WDR66, WNT6, ZDHHC18, ZNF382, and ZNF626.

Embodiments of the present disclosure also include novel differentially methylated regions (DMRs), each individually capable of distinguishing an oropharyngeal cancer (e.g., oropharyngeal squamous cell carcinoma (HPV(+)OPSCC)) and/or cervical squamous cell carcinoma (HPV(+) CSCC) from a control tissue sample (e.g., normal oropharyngeal tissue or normal cervical tissue). In accordance with these embodiments, the novel DMR(s) is from a gene selected from ABCB1, ARHGAP12, ASCL1, Clorf114, EMX1, GRIN2D, LOC645323, MAX.chr6.58147682-58147771, MAX.chr9.36739811-36739868, NEUROG3, NID2, TBX15, TMEM200C, TSPYL5, TTYH1, VWC2, ZNF610, ZNF69, ZNF773, and ZNF781.

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., tonsil tissue controls). In accordance with these embodiments, the novel DMR(s) is from a gene selected from ALX4, ATP10A, Clorf114, C1QL3, CA8, CACNA1A, CACNG8, CALCA, CCNA1, CLIC6, CLSTN2, CR1, CTNND2, DAB1, DGKG, DOK1, DOK6, DPP4, DUXA, ELMO1, EMBP1, EPDR1, FGF12, FLJ43390, FMN2, FOXB2, FOXD4, FREM3, GALR1, GDF6, GFRA1, GRIK3, HOXB3, HOXB4, HPSE2, LDLRAD2, LHX2, LOC100131366, LOC345643, LOC386758, LOC645323, LOC648809, LOC728392, MAML3, MAPRE2, MAX.chr1.226288154-226288189, MAX.chr1.2375078-2375126, MAX.chr1.241587339-241587784, MAX.chr1.50798781-50799423, MAX.chr10.22765150-22765477, MAX.chr10.23462342-23462436, MAX.chr11.14926602-14927044, MAX.chr11.58903531-58903592, MAX.chr13.28527984-28528214, MAX.chr13.29106641-29107037, MAX.chr14.100784488-100784782, MAX.chr16.3221176-3221223, MAX.chr16.3222040-3222098, MAX.chr16.71460171-71460282, MAX.chr19.11805263-11805639, MAX.chr19.16394457-16394646, MAX.chr19.21657626-21657769, MAX.chr19.22034646-22034887, MAX.chr19.23299989-23300156, MAX.chr19.30713427-30713588, MAX.chr19.30716926-30717074, MAX.chr19.30718373-30719719, MAX.chr2.118981724-118982174, MAX.chr2.127783107-127783403, MAX.chr2.173099712-173099791, MAX.chr2.66808635-66808731, MAX.chr22.50064113-50064259, MAX.chr3.137489884-137490061, MAX.chr5.138923141-138923219, MAX.chr5.42995180-42995535, MAX.chr6.38683091-38683226, MAX.chr7.121952014-121952084, MAX.chr7.155166980-155167310, MAX.chr8.99986792-99986864, MAX.chr9.79627078-79627116, MAX.chr9.79638034-79638077, MAX.chr9.98789824-98789847, MDFI, MECOM, MED12L, MIR129-2, MIR196A1, NELL1, NPY, ONECUT2, OPCML, PARP15, PDGFD, PEX5L, PRR15, SEMA6A, SFMBT2, SGIP1, SIM2, SLC35F3, SLCO4C1, SORCS3, ST6GALNAC5, ST8SIA5, SV2C, TACC2, TFAP2E, TLX2, TLX3, TRH, TRIM58, VAV3, VSTM2B, WDR17, ZNF254, ZNF43, ZNF486, ZNF491, ZNF518B, ZNF542, ZNF625, ZNF665, ZNF671, ZNF763, and ZNF844.

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal buffy coat controls). In accordance with these embodiments, the novel DMR(s) is from a gene selected from AGRN, ANKRD35, ARHGAP27, ARHGAP30, BCL2L11, BIN2, C10orf114, C4orf31, C6orf132, C6orf186, CCDC88B, CRHBP, DAPK1, DNMT3A, DPP10, ELMO1, EPDR1, FAM19A2, FLJ45983, FOSL1, FOXB1, GREM1, HMHA1, HOXA9, IFFO1, INPP4B, ITGB2, ITGB4, ITPKB, KCNIP2, KLHDC7B, LAT, LHX6, LIMK1, LOC100128239, LOC100192379, LOC646278, MAP2K2, MAX.chr1.210426156-210426257, MAX.chr1.84326495-84326656, MAX.chr10.119312785-119312882, MAX.chr15.67326025-67326060, MAX.chr16.54316401-54316453, MAX.chr16.85482306-85482494, MAX.chr17.74994454-74994572, MAX.chr17.76339840-76339972, MAX.chr2.7571082-7571136, MAX.chr21.45577347-45577679, MAX.chr3.14852538-14852568, MAX.chr3.187676564-187676668, MAX.chr4.174430662-174430793, MAX.chr5.177411809-177411836, MAX.chr6.45631561-45631625, MAX.chr7.25892382-25892451, MAX.chr7.402563-402641, MAX.chr7.64349554-64349606, MAX.chr8.142046239-142046398, MAX.chr8.145900842-145901246, MAX.chr9.126101804-126101848, MAX.chr9.126978999-126979182, MAX.chr9.36458633-36458725, MAX.chr9.87905315-87905326, MBP, MFNG, MT1A, MT1IP, NCOR2, NFATC1, NKX3-2, NRN1, OLIG1, PALLD, PAPLN, PDLIM2, PKN1, PRDM14, PRKG1, PRMT7, PTGER2, PTK2B, RAD52, RBM38, RHOF, RNF220, RTN4RL1, RXRA, SDCCAG8, SHROOM1, SKI, SLC12A8, SLC25A47, SPEG, SUCLG2, TBC1D10C, TMEM132E, VIPR2, WDR66, WNT6, ZDHHC18, ZNF382, and ZNF626.

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue controls). In accordance with these embodiments, the novel DMR(s) is from a gene selected from ALX4, Clorf114, CA8, CCNA1, CLSTN2, CR1, DAB1, DOK1, EMBP1, EPDR1, FLJ43390, FMN2, GDF6, GFRA1, HOXB3, LDLRAD2, LOC648809, MAPRE2, MAX.chr1.241587339-241587784, MAX.chr1.50798781-50799423, MAX.chr13.28527984-in 28528214, MAX.chr16.3221176-3221223, MAX.chr19.11805263-11805639, MAX.chr19.22034646-22034887, MAX.chr19.30718373-30719719, MAX.chr2.173099712-173099791, MAX.chr2.66808635-66808731, MAX.chr6.38683091-38683226, MAX.chr9.79638034-79638077, MECOM, ONECUT2, PARP15, SGIP1, SIM2, SORCS3, ST6GALNAC5, ST8SIA5, TFAP2E, TLX2, TLX3, VSTM2B, WDR17, ZNF254, ZNF43, ZNF491, ZNF763, and ZNF844.

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal buffy coat controls). In accordance with these embodiments, the novel DMR(s) is from a gene selected from FAM19A2, IFFO1, ITGB4, LOC100192379, MAX.chr1.84326495-84326656, MAX.chr16.85482306-85482494, MAX.chr6.45631561-45631625, MAX.chr7.25892382-25892451, MT1IP, NCOR2, OLIG1, RAD52, SHROOM1, SLC12A8, and TBC1D10C.

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue or normal buffy coat controls). In accordance with these embodiments, the novel DMR(s) is from a gene selected from MAX.chr19.30718373-30719719, ITGB4, MAX.chr7.25892382-25892451, RAD52, SHROOM1, SLC12A8, and TBC1D10C.

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue or normal buffy coat controls). In accordance with these embodiments, the novel DMR(s) is from a gene selected from ALX4, Clorf114, CA8, CCNA1, CLSTN2, CR1, DAB1, DOK1, EMBP1, EPDR1, FAM19A2, FLJ43390, FMN2, GDF6, GFRA1, HOXB3, IFFO1, ITGB4, LDLRAD2, LOC100192379, LOC648809, MAPRE2, MAX.chr1.241587339-241587784, MAX.chr1.50798781-50799423, MAX.chr1.84326495-84326656, MAX.chr13.28527984-28528214, MAX.chr16.3221176-3221223, MAX.chr16.85482306-85482494, MAX.chr19.11805263-11805639, MAX.chr19.22034646-22034887, MAX.chr19.30718373-30719719, MAX.chr2.173099712-173099791, MAX.chr2.66808635-66808731, MAX.chr6.38683091-38683226, MAX.chr6.45631561-45631625, MAX.chr7.25892382-25892451, MAX.chr9.79638034-79638077, MECOM, MT1IP, NCOR2, OLIG1, ONECUT2, PARP15, RAD52, SGIP1, SHROOM1, SIM2, SLC12A8, SORCS3, ST6GALNAC5, ST8SIA5, TBC1D10C, TFAP2E, TLX2, TLX3, VSTM2B, WDR17, ZNF254, ZNF43, ZNF491, ZNF763, and ZNF844.

Embodiments of the present disclosure also include novel differentially methylated regions (DMRs), each individually capable of distinguishing an oropharyngeal cancer (e.g., HPV⁺ oropharyngeal squamous cell cancer (HPV OPSCC)) from control or benign tissue (e.g., normal tissue or normal buffy coat controls). In accordance with these embodiments, the novel DMR(s) is from a gene selected from CA8, EMBP1, HOXB3, IFFO1, ITGB4, LOC100192379, LOC648809, MAX.chr1.84326495-84326656, MAX.chr16.3221176-3221223, MAX.chr16.85482306-85482494, MAX.chr19.30718373-30719719, MAX.chr9.79638034-79638077, MT1IP, ONECUT2, SHROOM1, SIM2, SLC12A8, TLX3, and ZNF763.

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue or normal buffy coat controls). In accordance with these embodiments, the novel DMR(s) is from a gene selected from Clorf114, CA8, CCNA1, EMBP1, EPDR1, FAM19A2, FMN2, HOXB3, IFFO1, ITGB4, LDLRAD2, LOC100192379, LOC648809, MAPRE2, MAX.chr1.50798781-50799423, MAX.chr1.84326495-84326656, MAX.chr16.3221176-3221223, MAX.chr19.11805263-11805639, MAX.chr2.66808635-66808731, MAX.chr6.38683091-38683226, MAX.chr6.45631561-45631625, MAX.chr9.79638034-79638077, MECOM, MT1IP, ONECUT2, PARP15, SHROOM1, SIM2, SLC12A8, SORCS3, ST6GALNAC5, ST8SIA5, TBC1D10C, TLX3, ZNF254, ZNF491, ZNF763, and ZNF844.

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue or normal buffy coat controls). In accordance with these embodiments, the novel DMR(s) is from a gene selected from CA8, EMBP1, HOXB3, IFFO1, ITGB4, LOC100192379, LOC648809, MAX.chr1.84326495-84326656, MAX.chr16.3221176-3221223, MAX.chr9.79638034-79638077, MT1IP, ONECUT2, SHROOM1, SIM2, SLC12A8, TLX3, and ZNF763.

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) in a saliva sample from a subject from control or benign tissue (e.g., saliva control samples). In accordance with these embodiments, the novel DMR(s) is from a gene selected from TLX3, MAX.chr16.3221176-3221223, TBC1D10C, and SHROOM1.

In some embodiments, the novel DMR(s) capable of distinguishing an oropharyngeal cancer from a control sample were validated using at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR, and based on at least one of an area under a ROC curve (AUC), fold-change in methylation, methylation percentage, and/or hypermethylation ratio between a test sample and a control sample.

In accordance with the above, a control sample comprises a sample from a subject that does not have cancer, a sample from a subject that does not have an oropharyngeal cancer, a sample from a subject that has a type of cancer that is not an oropharyngeal cancer, or a sample from a subject that has an HPV(+) cancer that is not an oropharyngeal cancer. In some embodiments, the control sample is from a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, or a stool sample. In some embodiments, the control sample is from an oropharyngeal tissue sample comprising one or more of soft palate cells or tissues, throat cells or tissues, tongue cells or tissues, and tonsil cells or tissues. In some embodiments, the tissue sample is an HPV(+) tissue sample.

In some embodiments, the novel DMR(s) capable of distinguishing an oropharyngeal cancer from a control sample is associated with an area under a ROC curve (AUC) greater than or equal to 0.5, wherein the ROC curve discriminates between a subject having or suspected of having OPSCC and a control DNA sample. In some embodiments, the novel DMR(s) capable of distinguishing an oropharyngeal cancer from a control sample is associated with an area under a ROC curve (AUC) greater than or equal to 0.6, wherein the ROC curve discriminates between a subject having or suspected of having OPSCC and a control DNA sample. In some embodiments, the novel DMR(s) capable of distinguishing an oropharyngeal cancer from a control sample is associated with an area under a ROC curve (AUC) greater than or equal to 0.7, wherein the ROC curve discriminates between a subject having or suspected of having OPSCC and a control DNA sample. In some embodiments, the novel DMR(s) capable of distinguishing an oropharyngeal cancer from a control sample is associated with an area under a ROC curve (AUC) greater than or equal to 0.8, wherein the ROC curve discriminates between a subject having or suspected of having OPSCC and a control DNA sample. In some embodiments, the novel DMR(s) capable of distinguishing an oropharyngeal cancer from a control sample is associated with an area under a ROC curve (AUC) greater than or equal to 0.9, wherein the ROC curve discriminates between a subject having or suspected of having OPSCC and a control DNA sample.

In some embodiments, the novel DMR(s) capable of distinguishing an oropharyngeal cancer from a control sample comprises an increased methylation percentage as compared to a control DNA sample. In some embodiments, the novel DMR(s) capable of distinguishing an oropharyngeal cancer from a control sample comprises an increased hypermethylation ratio as compared to a control DNA sample.

In some embodiments, the biological sample is obtained from the subject, and the method further comprises extracting the DNA sample from the biological sample. In some embodiments, the biological sample is collected with a collection device having an absorbing element capable of collecting the biological sample upon contact. In some embodiments, the biological sample is collected with a collection device having an extraction element capable of extracting the biological sample. In some embodiments, the absorbing element or extraction element is configured for insertion into an orifice (mouth, nose, or throat).

In some embodiments, the reagent that modifies DNA in a methylation-specific manner is a borane reducing agent. In some embodiments, the reagent that modifies DNA in a methylation-specific manner comprises one or more of a methylation-sensitive restriction enzyme, a methylation-dependent restriction enzyme, and a bisulfite reagent.

In some embodiments, determining the methylation profile of at least one DMR comprises amplifying at least a portion of the DMR using a set of primers (e.g., Tables 3 and 12). In some embodiments, determining the methylation profile of at least one DMR comprises performing at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR. In some embodiments, determining the methylation profile of at least one DMR comprises determining the presence or absence of methylation at a CpG site. In some embodiments, the one or more CpG sites are present in a coding region, a non-coding region, and/or a regulatory region of a gene (e.g., any one of the genes disclosed herein).

Embodiments of the present disclosure also include a method of identifying an oropharyngeal cancer. In accordance with these embodiments, the method includes determining a methylation profile in at least one differentially methylated region (DMR) of a DNA sample obtained from a subject having or suspected of having an oropharyngeal cancer by treating the sample with a reagent that modifies DNA in a methylation-specific manner. In some embodiments, the methylation profile indicates that the subject has an oropharyngeal cancer (e.g., HPV⁺ oropharyngeal squamous cell cancer (HPV⁺ OPSCC)). In some embodiments, the method further includes treating the subject with an anti-cancer therapy.

### BRIEF DESCRIPTION OF THE DRAWINGS

FIG. 1: Representative boxplot illustrating the ability of a DMR from GRIND2 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

10027| FIG. 2: Representative boxplot illustrating the ability of a DMR from EMX1 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 3: Representative boxplot illustrating the ability of a DMR from VWC2 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 4: Representative boxplot illustrating the ability of a DMR from ZNF610 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 5: Representative boxplot illustrating the ability of a DMR from ZNF781.A to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

10031| FIG. 6: Representative boxplot illustrating the ability of a DMR from TBX15 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 7: Representative boxplot illustrating the ability of a DMR from TSPYL5 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 8: Representative boxplot illustrating the ability of a DMR from LOC645323 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 9: Representative boxplot illustrating the ability of a DMR from ASCL1 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 10: Representative boxplot illustrating the ability of a DMR from ABCB1 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 11: Representative boxplot illustrating the ability of a DMR from ZNF69 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 12: Representative boxplot illustrating the ability of a DMR from MAX.chr9.36739811-36739868 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 13: Representative boxplot illustrating the ability of a DMR from ARHGAP12 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 14: Representative boxplot illustrating the ability of a DMR from Clorf114 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 15: Representative boxplot illustrating the ability of a DMR from MAX.chr6.58147682-58147771 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 16: Representative boxplot illustrating the ability of a DMR from NEUROG3 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 17: Representative boxplot illustrating the ability of a DMR from NID2 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 18: Representative boxplot illustrating the ability of a DMR from TMEM200C to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 19: Representative boxplot illustrating the ability of a DMR from TTYH1 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 20: Representative boxplot illustrating the ability of a DMR from ZNF773 to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 21: Representative boxplot illustrating the ability of a DMR from ZNF781.B to distinguish oropharyngeal cancer (HPV(+) oropharyngeal squamous cell carcinoma (OSPCC)) from controls (normal oropharyngeal tissue (NOP) and normal cervical tissue (NCS)), and HPV(+) cervical squamous cell carcinoma (CSCC).

FIG. 22: Representative boxplot of β-actin as a control for FIGS. 1-21.

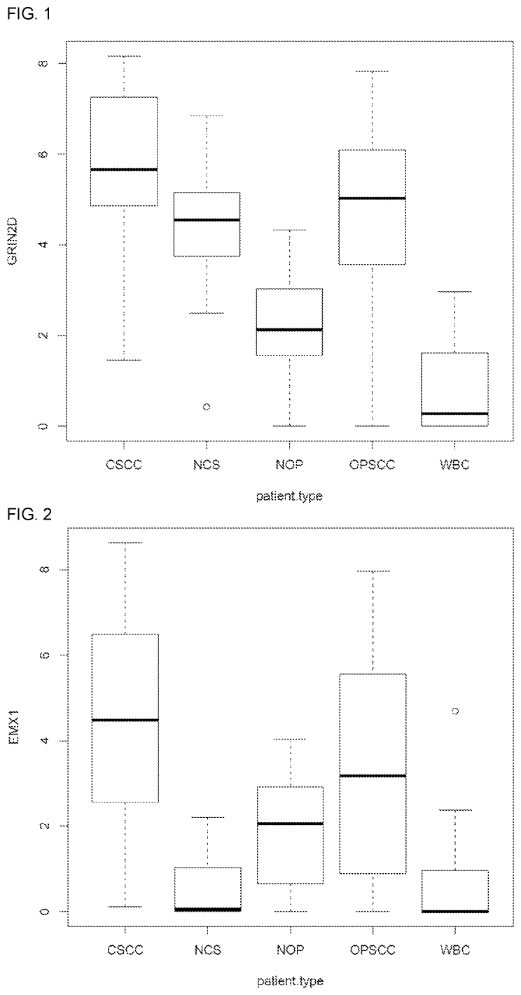

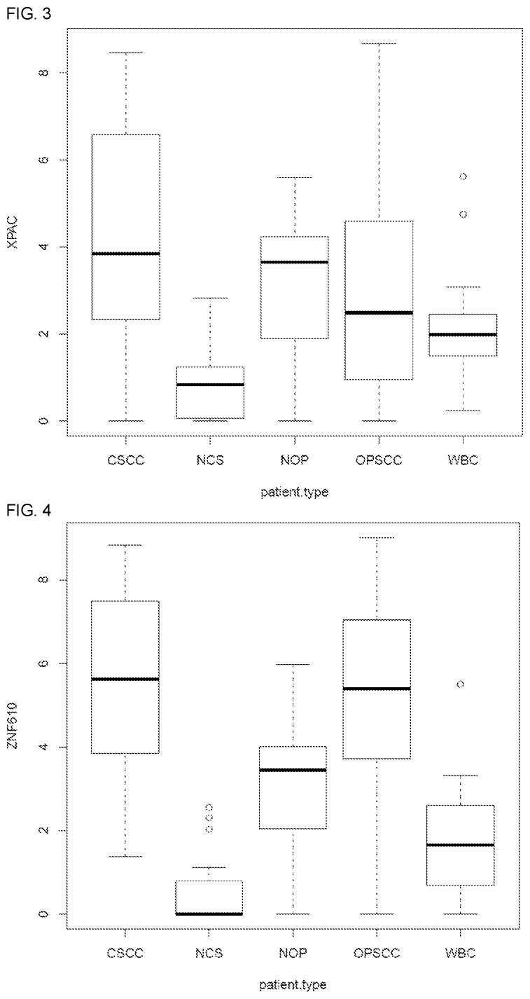

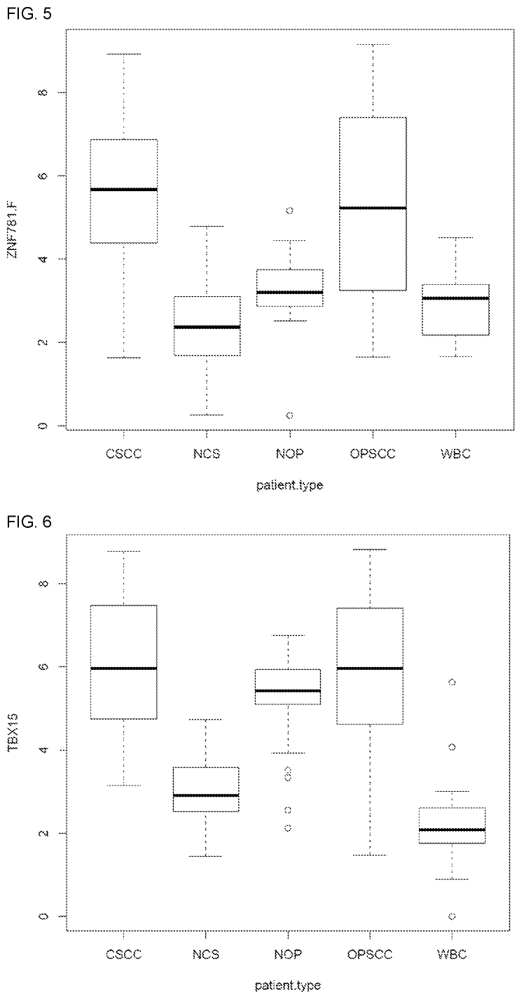

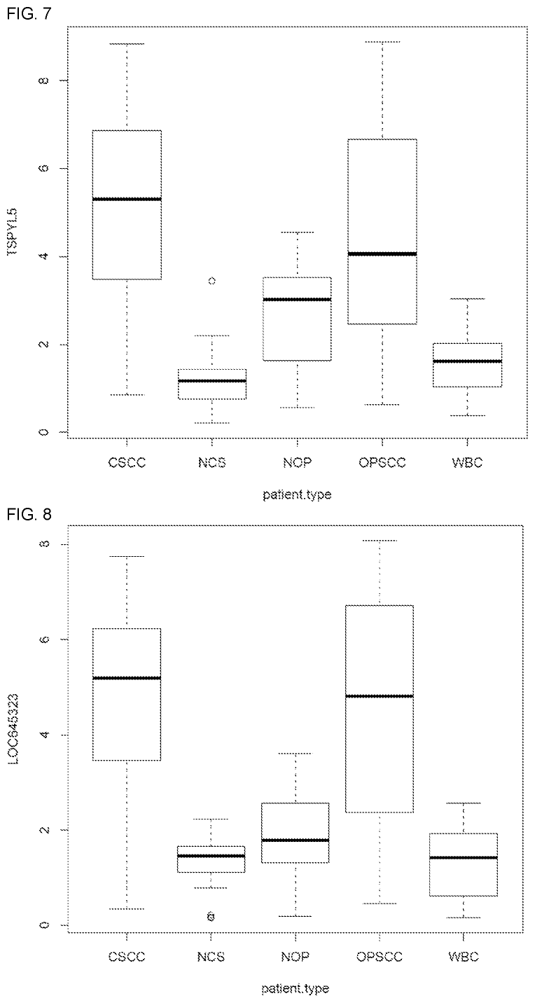

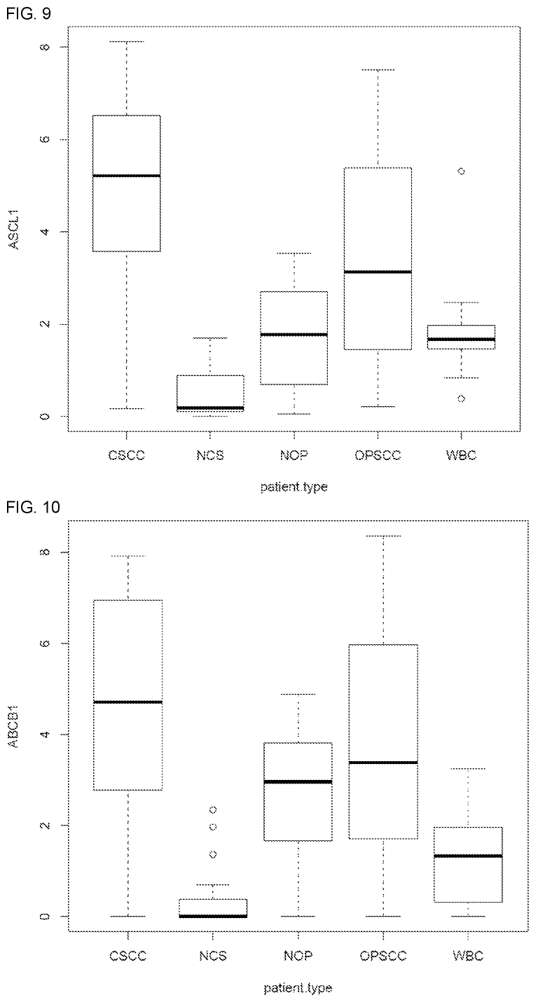

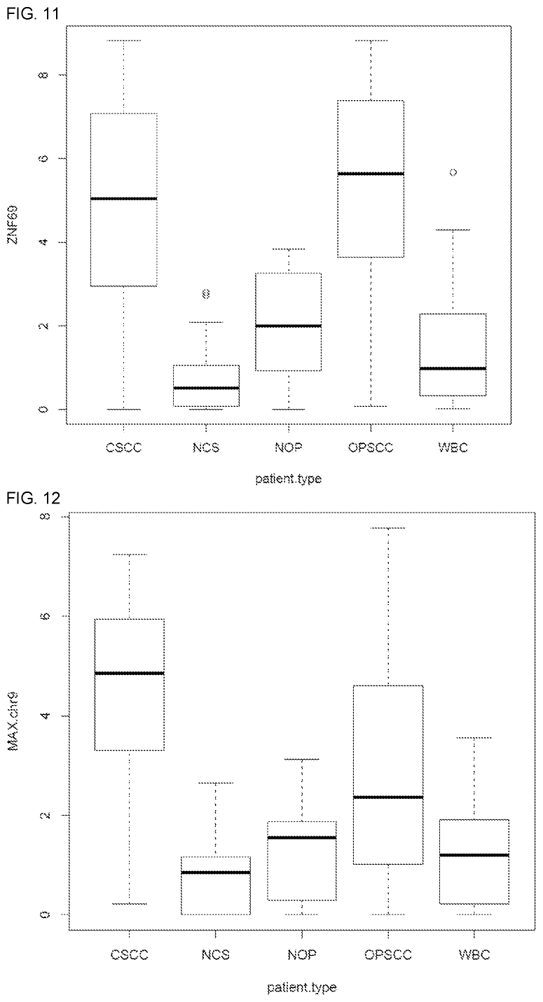

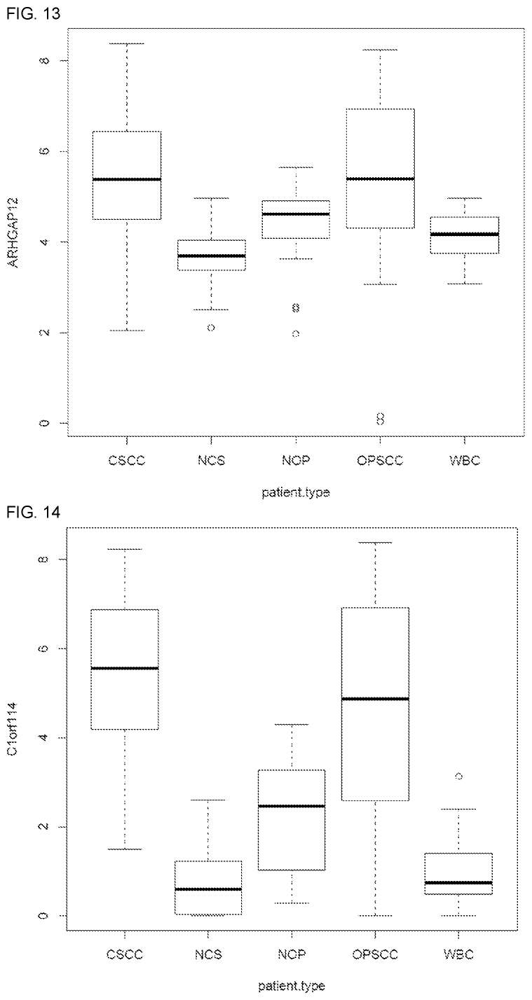

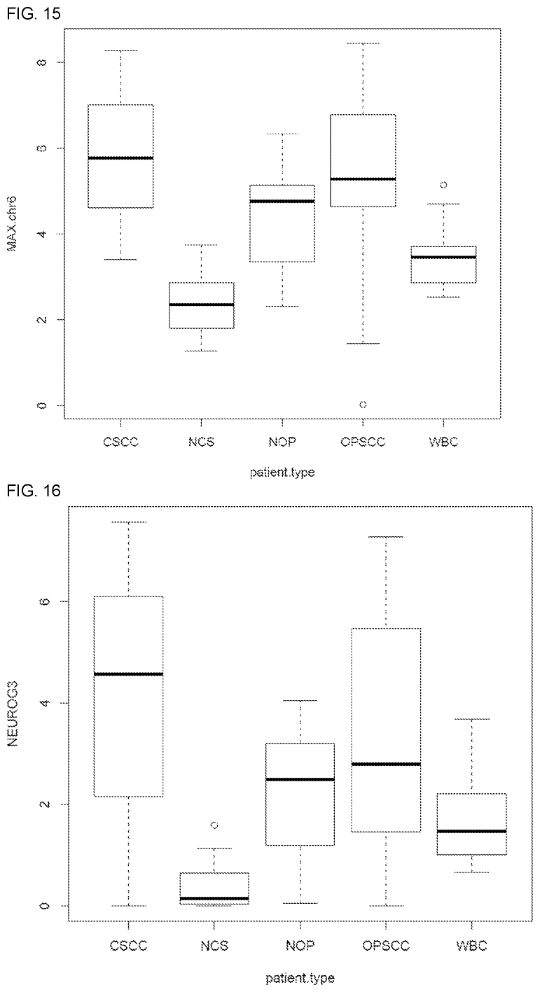

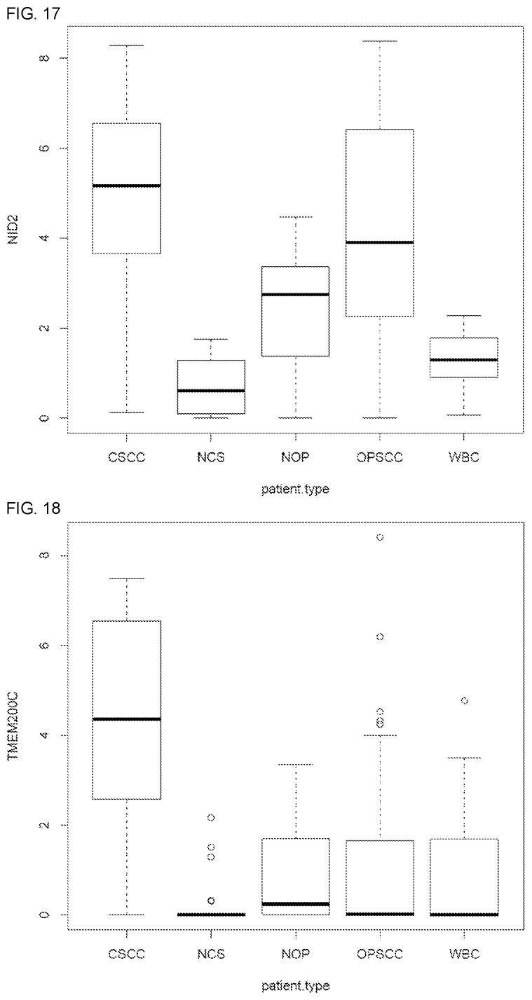

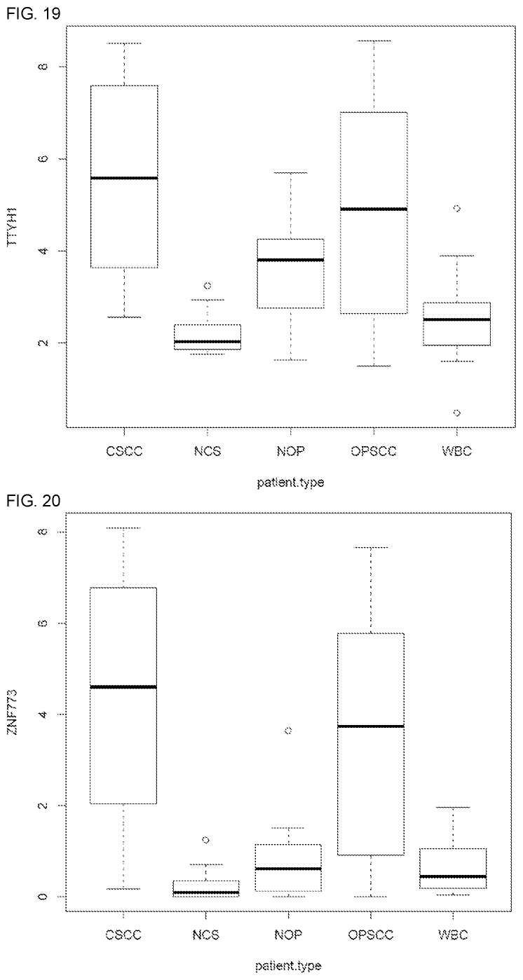

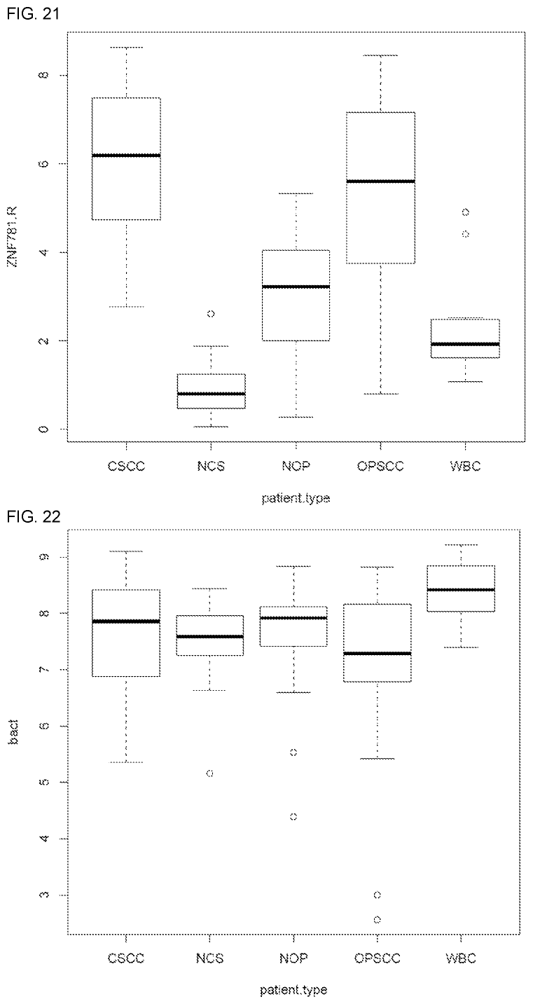

### DETAILED DESCRIPTION

Oropharyngeal (or head and neck) cancers account for 3% of the cancers diagnosed each year in the US and are projected to kill approximately 11,000 people in 2021. While the incidence of HPV-cancers are relatively constant, the HPV+ cancer subtype incidence is increasing. There is an urgent need for a non-invasive molecular test to screen patients at increased risk for oropharyngeal cancer as it likely would decrease the burden of this disease and save lives. At the present time, there are no accurate, user-friendly, and widely accessible screening tools for ideal clinical management of oropharyngeal tumors. To address these clinical gaps, experiments were conducted to develop a new approach anchored on marker detection in tissue, across multiple tissue compartments and in bodily fluid to target novel, highly discriminant methylated DNA markers with capacity to predict features of the primary tumor using an exquisitely sensitive analytical platform.

Accordingly, the various experiments described herein were conducted to discover novel methylated DNA markers in tissue for malignant oropharyngeal tumors by unbiased whole methylome sequencing (RRBS) and validate top candidates in independent tissue, to assess detection accuracy for oropharyngeal cancers by assay of top methylated DNA markers in plasma, to identify detection accuracy for oropharyngeal cancers by assay of top methylated DNA markers in bodily fluid, and to identify methylated DNA markers with potential oropharyngeal site-specificity by in silico comparison of discovered candidates against a whole methylome database created for neoplasms across multiple organs.

New molecular technologies offer the opportunity to re-imagine how cancer screening might be conducted. With sufficiently discriminant markers and highly sensitive assay tools, detection of multiple cancers at earliest stages by a blood or other distant media such as urine or saliva for oropharyngeal tumors may now be possible. As such, the traditional approach of single organ screening could give way to a new paradigm of multi-organ cancer screening using a single noninvasive test. The potential gains in cost-efficiency and the benefits in reducing the cancer death toll could be staggering. Blood testing represents the most attractive approach to universal cancer screening. However, blood test methods throughout the years have largely failed to detect early-stage cancers with adequate sensitivity or specificity to achieve clinical practicality or effectiveness. Historically, blood tests for cancer have focused on the clear compartment (i.e., plasma or serum) and have targeted various proteins or acquired genetic alterations associated with cancer. Such markers have often lacked site specificity, which creates diagnostic ambiguity and confuses the down-stream clinical evaluation.

As described further herein, the various embodiments of the present disclosure provide solutions to these technical and biological barriers. Analytical sensitivity has been increased by orders of magnitude over historical methods to be within the requisite zone for detection of low abundance markers with early-stage disease. Additionally, the data provided herein demonstrate that assay of markers provide complementary value to plasma testing alone for detection of earliest stage lesions, as alternative compartments may address other mechanisms of marker entry into blood.

Section headings as used in this section and the entire disclosure herein are merely for organizational purposes and are not intended to be limiting.

### 1. DEFINITIONS

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

The term “one or more”, as used herein, refers to a number higher than one. For example, the term “one or more” encompasses any of the following: two or more, three or more, four or more, five or more, six or more, seven or more, eight or more, nine or more, ten or more, twelve or more, thirteen or more, fourteen or more, fifteen or more, twenty or more, fifty or more, 100 or more, or an even greater number.

The term “one or more but less than a higher number,” “two or more but less than a higher number,” “three or more but less than a higher number,” “four or more but less than a higher number,” “five or more but less than a higher number,” “six or more but less than a higher number,” “seven or more but less than a higher number,” “eight or more but less than a higher number,” “nine or more but less than a higher number,” “ten or more but less than a higher number,” “eleven or more but less than a higher number,” “twelve or more but less than a higher number,” “thirteen or more but less than a higher number,” “fourteen or more but less than a higher number,” or “fifteen or more but less than a higher number” is not limited to a higher number. For example, the higher number can be 10,000, 1,000, 100, 50, etc. For example, the higher number can be approximately 50 (e.g., 50, 49, 48, 47, 46, 45, 44, 43, 42, 41, 40, 39, 38, 37, 36, 35, 34, 33, 32, 31, 32, 30, 29, 28, 27, 26, 25, 24, 23, 22, 21, 20, 19, 18, 17, 16, 15, 14, 13, 12, 11, 10, 9, 8, 7, 6, 5, 4, 3 or 2).

The term “one or more methylated markers” or “one or more DMRs” or “one or more genes” or “one or more markers” or “a plurality of methylated markers” or “a plurality of markers” or “a plurality of genes” or “a plurality of DMRs” is similarly not limited to a particular numerical combination. Indeed, any numerical combination of methylated markers is contemplated (e.g., 1-2 methylated markers, 1-3, 1-4, 1-5. 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38) (e.g., 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38) (e.g., 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38) (e.g., 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 4-25, 4-26, 4-27, 4-28, 4-29, 4-30, 4-31, 4-32, 4-33, 4-34, 4-35, 4-36, 4-37, 4-38) (e.g., 5-6, 5-7, 5-8, 5-9, 5-10, 5-11, 5-12, 5-13, 5-14, 5-15, 5-16, 5-17, 5-18, 5-19, 5-20, 5-21, 5-22, 5-23, 5-24, 5-25, 5-26, 5-27, 5-28, 5-29, 5-30, 5-31, 5-32, 5-33, 5-34, 5-35, 5-36, 5-37, 5-38) (e.g., 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 6-25, 6-26, 6-27, 6-28, 6-29, 6-30, 6-31, 6-32, 6-33, 6-34, 6-35, 6-36, 6-37, 6-38) (e.g., 7-8, 7-9, 7-10, 7-11, 7-12, 7-13, 7-14, 7-15, 7-16, 7-17, 7-18, 7-19, 7-20, 7-21, 7-22, 7-23, 7-24, 7-25, 7-26, 7-27, 7-28, 7-29, 7-30, 7-31, 7-32, 7-33, 7-34, 7-35, 7-36, 7-37, 7-38) (e.g., 8-9, 8-10, 8-11, 8-12, 8-13, 8-14, 8-15, 8-16, 8-17, 8-18, 8-19, 8-20, 8-21, 8-22, 8-23, 8-24, 8-25, 8-26, 8-27, 8-28, 8-29, 8-30, 8-31, 8-32, 8-33, 8-34, 8-35, 8-36, 8-37, 8-38) (e.g., 9-10, 9-11, 9-12, 9-13, 9-14, 9-15, 9-16, 9-17, 9-18, 9-19, 9-20, 9-21, 9-22, 9-23, 9-24, 9-25, 9-26, 9-27, 9-28, 9-29, 9-30, 9-31, 9-32, 9-33, 9-34, 9-35, 9-36, 9-37, 9-38) (e.g., 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 10-31, 10-32, 10-33, 10-34, 10-35, 10-36, 10-37, 10-38) (e.g., 11-12, 11-13, 11-14, 11-15, 11-16, 11-17, 11-18, 11-19, 11-20, 11-21, 11-22, 11-23, 11-24, 11-25, 11-26, 11-27, 11-28, 11-29, 11-30, 11-31, 11-32, 11-33, 11-34, 11-35, 11-36, 11-37, 11-38) (e.g., 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 12-31, 12-32, 12-33, 12-34, 12-35, 12-36, 12-37, 12-38) (e.g., 13-14, 13-15, 13-16, 13-17, 13-18, 13-19, 13-20, 13-21, 13-22, 13-23, 13-24, 13-25, 13-26, 13-27, 13-28, 13-29, 13-30, 13-31, 13-32, 13-33, 13-34, 13-35, 13-36, 13-37, 13-38) (e.g., 14-15, 14-16, 14-17, 14-18, 14-19, 14-20, 14-21, 14-22, 14-23, 14-24, 14-25, 14-26, 14-27, 14-28, 14-29, 14-30, 14-31, 14-32, 14-33, 14-34, 14-35, 14-36, 14-37, 14-38) (e.g., 15-16, 15-17, 15-18, 15-19, 15-20, 15-21, 15-22, 15-23, 15-24, 15-25, 15-26, 15-27, 15-28, 15-29, 15-30, 15-31, 15-32, 15-33, 15-34, 15-35, 15-36, 15-37, 15-38) (e.g., 16-17, 16-18, 16-19, 16-20, 16-21, 16-22, 16-23, 16-24, 16-25, 16-26, 16-27, 16-28, 16-29, 16-30, 16-31, 16-32, 16-33, 16-34, 16-35, 16-36, 16-37, 16-38) (e.g., 17-18, 17-19, 17-20, 17-21, 17-22, 17-23, 17-24, 17-25, 17-26, 17-27, 17-28, 17-29, 17-30, 17-31, 17-32, 17-33, 17-34, 17-35, 17-36, 17-37, 17-38) (e.g., 18-19, 18-20, 18-21, 18-22, 18-23, 18-24, 18-25, 18-26, 18-27, 18-28, 18-29, 18-30, 18-31, 18-32, 18-33, 18-34, 18-35, 18-36, 18-37, 18-38) (e.g., 19-20, 19-21, 19-22, 19-23, 19-24, 19-25, 19-26, 19-27, 19-28, 19-29, 19-30, 19-31, 19-32, 19-33, 19-34, 19-35, 19-36, 19-37, 19-38) (e.g., 20-21, 20-22, 20-23, 20-24, 20-25, 20-26, 20-27, 20-28, 20-29, 20-30, 20-31, 20-32, 20-33, 20-34, 20-35, 20-36, 20-37, 20-38) (e.g., 21-22, 21-23, 21-24, 21-25, 21-26, 21-27, 21-28, 21-29, 21-30, 21-31, 21-32, 21-33, 21-34, 21-35, 21-36, 21-37, 21-38) (e.g., 22-23, 22-24, 22-25, 22-26, 22-27, 22-28, 22-29, 22-30, 22-31, 22-32, 22-33, 22-34, 22-35, 22-36, 22-37, 22-38) (e.g., 23-24, 23-25, 23-26, 23-27, 23-28, 23-29, 23-30, 23-31, 23-32, 23-33, 23-34, 23-35, 23-36, 23-37, 23-38) (e.g., 24-25, 24-26, 24-27, 24-28, 24-29, 24-30, 24-31, 24-32, 24-33, 24-34, 24-35, 24-36, 24-37, 24-38) (e.g., 25-26, 25-27, 25-28, 25-29, 25-30, 25-31, 25-32, 25-33, 25-34, 25-35, 25-36, 25-37, 25-38) (e.g., 26-27, 26-28, 26-29, 26-30, 26-31, 26-32, 26-33, 26-34, 26-35, 26-36, 26-37, 26-38) (e.g., 27-28, 27-29, 27-30, 27-31, 27-32, 27-33, 27-34, 27-35, 27-36, 27-37, 27-38) (e.g., 28-29, 28-30, 28-31, 28-32, 28-33, 28-34, 28-35, 28-36, 28-37, 28-38) (e.g., 29-30, 29-31, 29-32, 29-33, 29-34, 29-35, 29-36, 29-37, 29-38) (e.g., 30-31, 30-32, 30-33, 30-34, 30-35, 30-36, 30-37, 30-38) (e.g., 31-32, 31-33, 31-34, 31-35, 31-36, 31-37, 31-38) (e.g., 32-33, 32-34, 32-35, 32-36, 32-37, 32-38) (e.g., 33-34, 33-35, 33-36, 33-37, 33-38) (e.g., 34-35, 34-36, 34-37, 34-38) (e.g., 35-36, 35-37, 35-38) (e.g., 36-37, 36-38) (e.g., 37-38) (e.g., 38 or fewer; 37 or fewer; 36 or fewer; 35 or fewer; 34 or fewer; 33 or fewer; 32 or fewer; 31 or fewer; 30 or fewer; 29 or fewer; 28 or fewer; 27 or fewer; 26 or fewer; 25 or fewer; 24 or fewer; 23 or fewer; 22 or fewer; 21 or fewer; 20 or fewer; 19 or fewer; 18 or fewer; 17 or fewer; 16 or fewer; 15 or fewer; 14 or fewer; 13 or fewer; 12 or fewer; 11 or fewer; 10 or fewer; 9 or fewer; 8 or fewer; 7 or fewer; 6 or fewer; 5 or fewer; 4 or fewer; 3 or fewer; 2 or 1).

The term “one or more protein markers” is similarly not limited to a particular numerical combination. Indeed, any numerical combination of protein markers is contemplated (e.g., 1-2 protein markers, 1-3, 1-4, 1-5) (e.g., 2-3, 2-4, 2-5) (e.g., 3-4, 3-5) (e.g., 4-5) (e.g., 5 or fewer; 4 or fewer; 3 or fewer; 2 or 1).

The term “multiple types of cancer” or “one or more types of cancer” or “one or more subtypes of cancer” or “a plurality of different types or subtypes of cancer” is similarly not limited to a particular numerical combination. Any numerical combination of types or subtypes of oropharyngeal cancers can be identified using the DNA methylation markers of the present disclosure, including, but not limited to, HPV⁺ oropharyngeal squamous cell cancer.

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “wild-type” when made in reference to a protein refers to a protein that has the characteristics of a naturally occurring protein. The term “naturally-occurring” as applied to an object refers to the fact that an object can be found in nature. For example, a polypeptide or polynucleotide sequence that is present in an organism (including viruses) that can be isolated from a source in nature, and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population, or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method. In some embodiments, the primer pair is specific for a specific differentially methylated region (e.g., DMRs in Tables 1, 2, 6, and 7) and specifically binds at least a portion of a genetic region comprising the DMR (e.g., chromosomal coordinates in Tables 1, 2, 6, and 7).

The term “probe” refers to an oligonucleotide (e.g., a sequence of nucleotides), whether occurring naturally as in a purified restriction digest or produced synthetically, recombinantly, or by PCR amplification, which is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the embodiments of the present disclosure may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the various embodiment of the present disclosure be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence.

Accordingly, as used herein, “non-target”, e.g., as it is used to describe a nucleic acid such as a DNA, refers to nucleic acid that may be present in a reaction, but that is not the subject of detection or characterization by the reaction. In some embodiments, non-target nucleic acid may refer to nucleic acid present in a sample that does not, e.g., contain a target sequence, while in some embodiments, non-target may refer to exogenous nucleic acid, i.e., nucleic acid that does not originate from a sample containing or suspected of containing a target nucleic acid, and that is added to a reaction, e.g., to normalize the activity of an enzyme (e.g., polymerase) to reduce variability in the performance of the enzyme in the reaction.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

As used herein, the term “amplification reagents” refers to those reagents (deoxyribonucleoside triphosphates, buffer, etc.), needed for amplification except for primers, nucleic acid template, and the amplification enzyme. Typically, amplification reagents along with other reaction components are placed and contained in a reaction vessel.

As used herein, the term “control” when used in reference to nucleic acid detection or analysis refers to a nucleic acid having known features (e.g., known sequence, known copy-number per cell), for use in comparison to an experimental target (e.g., a nucleic acid of unknown concentration). A control may be an endogenous, preferably invariant gene against which a test or target nucleic acid in an assay can be normalized. Such normalizing controls for sample-to-sample variations that may occur in, for example, sample processing, assay efficiency, etc., and allows accurate sample-to-sample data comparison. Genes that find use for normalizing nucleic acid detection assays on human samples include, e.g., b-actin, ZDHHC1, and B3GALT6 (see, e.g., U.S. patents application Ser. Nos. 14/966,617 and 62/364,082, each incorporated herein by reference). As used herein “ZDHHC1” refers to a gene encoding a protein characterized as a zinc finger, DHHC-type containing 1, located in human DNA on Chr 16 (16q22.1) and belonging to the DHHC palmitoyltransferase family.

Controls may also be external. For example, in quantitative assays such as qPCR, QuARTS, etc., a “calibrator” or “calibration control” is a nucleic acid of known sequence, e.g., having the same sequence as a portion of an experimental target nucleic acid, and a known concentration or series of concentrations (e.g., a serially diluted control target for generation of calibration curved in quantitative PCR). Typically, calibration controls are analyzed using the same reagents and reaction conditions as are used on an experimental DNA. In certain embodiments, the measurement of the calibrators is done at the same time, e.g., in the same thermal cycler, as the experimental assay. In preferred embodiments, multiple calibrators may be included in a single plasmid, such that the different calibrator sequences are easily provided in equimolar amounts. In some embodiments, plasmid calibrators are digested, e.g., with one or more restriction enzymes, to release calibrator portion from the plasmid vector. See, e.g., WO 2015/066695, which is included herein by reference.

As described further herein, a “control” or “control sample” can include, but is not limited to, a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, or a stool sample. In some embodiments, the control sample is from an oropharyngeal tissue sample comprising one or more of soft palate cells or tissues, throat cells or tissues, tongue cells or tissues, and tonsil cells or tissues. In some embodiments, a control sample is from a subject that does not have cancer, a sample from a subject that does not have an oropharyngeal cancer, a sample from a subject that has a type of cancer that is not an oropharyngeal cancer, or a sample from a subject that has an HPV(+) cancer that is not an oropharyngeal cancer. In some embodiments, the tissue sample is an HPV(+) tissue sample.

As used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence or absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

As used herein, the term “methylation level” as applied to a methylation marker refers to the amount of methylation within a particular methylation marker. Methylation level may also refer to the amount of methylation within a particular methylation marker in comparison with an established norm or control. Methylation level may also refer to whether one or more cytosine residues present in a CpG context have or do not have a methylation group. Methylation level may also refer to the fraction of cells in a sample that do or do not have a methylation group on such cytosines. Methylation level may also alternatively describe whether a single CpG di-nucleotide is methylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.). A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids or by comparing TET-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

The term “methylation score” as used herein is a score indicative of detected methylation events in a marker or panel of markers in comparison with median methylation events for the marker or panel of markers from a random population of mammals (e.g., a random population of 10, 20, 30, 40, 50, 100, or 500 mammals) that do not have a specific neoplasm of interest. An elevated methylation score in a marker or panel of markers can be any score provided that the score is greater than a corresponding reference score. For example, an elevated score of methylation in a marker or panel of markers can be 0.5, 1, 2, 3, 4, 5, 6, 7, 8, 9, 10, or more fold greater than the reference methylation score.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who do not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g., Antequera et al. (1990) Cell 62:503-514).

As used herein, a “CpG island” or “cytosine-phosphate-guanine island”) refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196:261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97:5237-5242; Salmon and Kaye (1970) Biochim. Biophys. Acta. 204:340-351; Grafstrom (1985) Nucleic Acids Res. 13:2827-2842; Nyce (1986) Nucleic Acids Res. 14:4353-4367; Woodcock (1987) Biochem. Biophys. Res. Commun. 145:888-894).

As used herein, a “methylation-specific reagent” refers to a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. Examples of such reagents include, but are not limited to, a methylation-sensitive restriction enzyme, a methylation-dependent restriction enzyme, a bisulfite reagent, a TET enzyme, and a borane reducing agent.

A change in the nucleic acid nucleotide sequence by a methylation-specific reagent can also result in a nucleic acid molecule in which each methylated nucleotide is modified to a different nucleotide.

The term “methylation assay” refers to any assay for determining the methylation state of one or more CpG dinucleotide sequences within a sequence of a nucleic acid.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, as described by Gonzalgo et al. (1997) Cancer Research 57:594-599.

The term “MethyLight™” refers to the art-recognized fluorescence-based real-time PCR technique described by Eads et al. (1999) Cancer Res. 59:2302-2306.

The term “HeavyMethyl™” refers to an assay wherein methylation specific blocking probes (also referred to herein as blockers) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones (1997) Nucleic Acids Res. 25:2529-2531.

The term “MSP” (Methylation-specific PCR) refers to the art-recognized methylation assay described by Herman et al. (1996) Proc. Natl. Acad. Sci. USA 93:9821-9826, and by U.S. Pat. No. 5,786,146.

The term “COBRA” (Combined Bisulfite Restriction Analysis) refers to the art-recognized methylation assay described by Xiong & Laird (1997) Nucleic Acids Res. 25:2532-2534.

The term “MCA” (Methylated CpG Island Amplification) refers to the methylation assay described by Toyota et al. (1999) Cancer Res. 59:2307-12, and in WO 00/26401A1.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease), and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

The term “AUC” as used herein is an abbreviation for the “area under a curve”. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better; the optimum is 1; a random test would have a ROC curve lying on the diagonal with an area of 0.5; for reference: J. P. Egan. (1975) Signal Detection Theory and ROC Analysis, Academic Press, New York).

The term “neoplasm” as used herein refers to any new and abnormal growth of tissue. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

As used herein, the term “adenoma” refers to a benign tumor of glandular origin. Although these growths are benign, over time they may progress to become malignant.

The term “pre-cancerous” or “pre-neoplastic” and equivalents thereof refer to any cellular proliferative disorder that is undergoing malignant transformation.

A “site” of a neoplasm, adenoma, cancer, etc. is the tissue, organ, cell type, anatomical area, body part, etc. in a subject's body where the neoplasm, adenoma, cancer, etc. is located.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject contracting a neoplasm or the likelihood that such a subject will respond favorably to a compound (e.g., a pharmaceutical, e.g., a drug) or other treatment.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, be combined with other nucleic acids or molecules. For example, an isolated nucleic acid may be present in a host cell into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution containing salts (e.g., NaCl), detergents (e.g., SDS), and other components (e.g., Denhardt's solution, dry milk, salmon sperm DNA, etc.).

The term “sample” is used in its broadest sense. In one sense it can refer to an animal cell or tissue. In another sense, it refers to a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the various embodiments of the present disclosure.

As used herein, a “remote sample” as used in some contexts relates to a sample indirectly collected from a site that is not the cell, tissue, or organ source of the sample. For instance, when sample material originating from the pancreas is assessed in a stool sample the sample is a remote sample.

As used herein, the terms “patient” or “subject” refer to organisms to be subject to various tests described herein. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present disclosure provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including racehorses), and the like. Embodiments of the present disclosure further include a system for diagnosing one or more types or subtypes of oropharyngeal cancers in a subject. The system can be provided, for example, as a commercial kit that can be used to screen for a risk of one or more types or subtypes of oropharyngeal cancers or diagnose one or more types or subtypes of oropharyngeal cancers in a subject from whom a biological sample has been collected. An exemplary system provided in accordance with the various embodiments of present disclosure includes assessing the methylation state or profile of a marker, as described herein.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### 2. METHYLATED DNA MARKERS AND BIOMARKER PANELS

Embodiments of the present disclosure provide methods, compositions, and systems for screening one or more types of oropharyngeal cancer from a biological sample. In accordance with these embodiments, the present disclosure includes, but is not limited to, methods and compositions for detecting the presence of one or more types or subtypes of oropharyngeal cancer from a biological sample. In some embodiments, the biological sample is a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample. In some embodiments, the tissue sample is an oropharyngeal tissue sample comprising one or more of soft palate cells or tissues, throat cells or tissues, tongue cells or tissues, and tonsil cells or tissues. In some embodiments, the tissue sample is an HPV(+) tissue sample. In some embodiments, the subject is a human.

As described further herein, embodiments of the present disclosure include novel differentially methylated regions (DMRs), each individually capable of distinguishing an oropharyngeal cancer (e.g., HPV⁺ oropharyngeal squamous cell cancer (HPV⁺ OPSCC)) from a control sample (e.g., benign tissue, including but not limited to, oropharyngeal tissue, cervical tissue, tonsil tissue, a buffy coat sample, and a saliva sample). In accordance with these embodiments, the novel DMR(s) is from a gene selected from ABCB1, ARHGAP12, ASCL1, Clorf114, EMX1, GRIN2D, LOC645323, MAX.chr6.58147682-58147771, MAX.chr9.36739811-36739868, NEUROG3, NID2, TBX15, TMEM200C, TSPYL5, TTYH1, VWC2, ZNF610, ZNF69, ZNF773, ZNF781, ALX4, ATP10A, C1QL3, CA8, CACNA1A, CACNG8, CALCA, CCNA1, CLIC6, CLSTN2, CR1, CTNND2, DAB1, DGKG, DOK1, DOK6, DPP4, DUXA, ELMO1, EMBP1, EPDR1, FGF12, FLJ43390, FMN2, FOXB2, FOXD4, FREM3, GALR1, GDF6, GFRA1, GRIK3, HOXB3, HOXB4, HPSE2, LDLRAD2, LHX2, LOC100131366, LOC345643, LOC386758, LOC648809, LOC728392, MAML3, MAPRE2, MAX.chr1.226288154-226288189, MAX.chr1.2375078-2375126, MAX.chr1.241587339-241587784, MAX.chr1.50798781-50799423, MAX.chr10.22765150-22765477, MAX.chr10.23462342-23462436, MAX.chr11.14926602-14927044, MAX.chr11.58903531-58903592, MAX.chr13.28527984-28528214, MAX.chr13.29106641-29107037, MAX.chr14.100784488-100784782, MAX.chr16.3221176-3221223, MAX.chr16.3222040-3222098, MAX.chr16.71460171-71460282, MAX.chr19.11805263-11805639, MAX.chr19.16394457-16394646, MAX.chr19.21657626-21657769, MAX.chr19.22034646-22034887, MAX.chr19.23299989-23300156, MAX.chr19.30713427-30713588, MAX.chr19.30716926-30717074, MAX.chr19.30718373-30719719, MAX.chr2.118981724-118982174, MAX.chr2.127783107-127783403, MAX.chr2.173099712-173099791, MAX.chr2.66808635-66808731, MAX.chr22.50064113-50064259, MAX.chr3.137489884-137490061, MAX.chr5.138923141-138923219, MAX.chr5.42995180-42995535, MAX.chr6.38683091-38683226, MAX.chr7.121952014-121952084, MAX.chr7.155166980-155167310, MAX.chr8.99986792-99986864, MAX.chr9.79627078-79627116, MAX.chr9.79638034-79638077, MAX.chr9.98789824-98789847, MDFI, MECOM, MED12L, MIR129-2, MIR196A1, NELL1, NPY, ONECUT2, OPCML, PARP15, PDGFD, PEX5L, PRR15, SEMA6A, SFMBT2, SGIP1, SIM2, SLC35F3, SLCO4C1, SORCS3, ST6GALNAC5, ST8SIA5, SV2C, TACC2, TFAP2E, TLX2, TLX3, TRH, TRIM58, VAV3, VSTM2B, WDR17, ZNF254, ZNF43, ZNF486, ZNF491, ZNF518B, ZNF542, ZNF625, ZNF665, ZNF671, ZNF763, ZNF844, AGRN, ANKRD35, ARHGAP27, ARHGAP30, BCL2L11, BIN2, C10orf114, C4orf31, C6orf132, C6orf186, CCDC88B, CRHBP, DAPK1, DNMT3A, DPP10, FAM19A2, FLJ45983, FOSL1, FOXB1, GREM1, HMHA1, HOXA9, IFFO1, INPP4B, ITGB2, ITGB4, ITPKB, KCNIP2, KLHDC7B, LAT, LHX6, LIMK1, LOC100128239, LOC100192379, LOC646278, MAP2K2, MAX.chr1.210426156-210426257, MAX.chr1.84326495-84326656, MAX.chr10.119312785-119312882, MAX.chr15.67326025-67326060, MAX.chr16.54316401-54316453, MAX.chr16.85482306-85482494, MAX.chr17.74994454-74994572, MAX.chr17.76339840-76339972, MAX.chr2.7571082-7571136, MAX.chr21.45577347-45577679, MAX.chr3.14852538-14852568, MAX.chr3.187676564-187676668, MAX.chr4.174430662-174430793, MAX.chr5.177411809-177411836, MAX.chr6.45631561-45631625, MAX.chr7.25892382-25892451, MAX.chr7.402563-402641, MAX.chr7.64349554-64349606, MAX.chr8.142046239-142046398, MAX.chr8.145900842-145901246, MAX.chr9.126101804-126101848, MAX.chr9.126978999-126979182, MAX.chr9.36458633-36458725, MAX.chr9.87905315-87905326, MBP, MENG, MT1A, MT1IP, NCOR2, NFATC1, NKX3-2, NRN1, OLIG1, PALLD, PAPLN, PDLIM2, PKN1, PRDM14, PRKG1, PRMT7, PTGER2, PTK2B, RAD52, RBM38, RHOF, RNF220, RTN4RL1, RXRA, SDCCAG8, SHROOM1, SKI, SLC12A8, SLC25A47, SPEG, SUCLG2, TBC1D10C, TMEM132E, VIPR2, WDR66, WNT6, ZDHHC18, ZNF382, and ZNF626 (Table 1).

Embodiments of the present disclosure also include novel differentially methylated regions (DMRs), each individually capable of distinguishing an oropharyngeal cancer (e.g., oropharyngeal squamous cell carcinoma (HPV(+)OPSCC)) and/or cervical squamous cell carcinoma (HPV(+) CSCC) from a control tissue sample (e.g., normal oropharyngeal tissue or normal cervical tissue). In accordance with these embodiments, the novel DMR(s) is from a gene selected from ABCB1, ARHGAP12, ASCL1, Clorf114, EMX1, GRIN2D, LOC645323, MAX.chr6.58147682-58147771, MAX.chr9.36739811-36739868, NEUROG3, NID2, TBX15, TMEM200C, TSPYL5, TTYH1, VWC2, ZNF610, ZNF69, ZNF773, and ZNF781 (Table 2).

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., tonsil tissue controls). In accordance with these embodiments, the novel DMR(s) is from a gene selected from ALX4, ATP10A, Clorf114, C1QL3, CA8, CACNA1A, CACNG8, CALCA, CCNA1, CLIC6, CLSTN2, CR1, CTNND2, DAB1, DGKG, DOK1, DOK6, DPP4, DUXA, ELMO1, EMBP1, EPDR1, FGF12, FLJ43390, FMN2, FOXB2, FOXD4, FREM3, GALR1, GDF6, GFRA1, GRIK3, HOXB3, HOXB4, HPSE2, LDLRAD2, LHX2, LOC100131366, LOC345643, LOC386758, LOC645323, LOC728392, LOC648809, MAML3, MAPRE2, MAX.chr1.226288154-226288189, MAX.chr1.2375078-2375126, MAX.chr1.241587339-241587784, MAX.chr1.50798781-50799423, MAX.chr10.22765150-22765477, MAX.chr10.23462342-23462436, MAX.chr11.14926602-14927044, MAX.chr11.58903531-58903592, MAX.chr13.28527984-28528214, MAX.chr13.29106641-29107037, MAX.chr14.100784488-100784782, MAX.chr16.3221176-3221223, MAX.chr16.3222040-3222098, MAX.chr16.71460171-71460282, MAX.chr19.11805263-11805639, MAX.chr19.16394457-16394646, MAX.chr19.21657626-21657769, MAX.chr19.22034646-22034887, MAX.chr19.23299989-23300156, MAX.chr19.30713427-30713588, MAX.chr19.30716926-30717074, MAX.chr19.30718373-30719719, MAX.chr2.118981724-118982174, MAX.chr2.127783107-127783403, MAX.chr2.173099712-173099791, MAX.chr2.66808635-66808731, MAX.chr22.50064113-50064259, MAX.chr3.137489884-137490061, MAX.chr5.138923141-138923219, MAX.chr5.42995180-42995535, MAX.chr6.38683091-38683226, MAX.chr7.121952014-121952084, MAX.chr7.155166980-155167310, MAX.chr8.99986792-99986864, MAX.chr9.79627078-79627116, MAX.chr9.79638034-79638077, MAX.chr9.98789824-98789847, MDFI, MECOM, MED12L, MIR129-2, MIR196A1, NELL1, NPY, ONECUT2, OPCML, PARP15, PDGFD, PEX5L, PRR15, SEMA6A, SFMBT2, SGIP1, SIM2, SLC35F3, SLCO4C1, SORCS3, ST6GALNAC5, ST8SIA5, SV2C, TACC2, TFAP2E, TLX2, TLX3, TRH, TRIM58, VAV3, VSTM2B, WDR17, ZNF254, ZNF43, ZNF486, ZNF491, ZNF518B, ZNF542, ZNF625, ZNF665, ZNF671, ZNF763, and ZNF844 (Table 6).

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal buffy coat controls). In accordance with these embodiments, the novel DMR(s) is from a gene selected from AGRN, ANKRD35, ARHGAP27, ARHGAP30, BCL2L11, BIN2, C10orf114, C4orf31, C6orf132, C6orf186, CCDC88B, CRHBP, DAPK1, DNMT3A, DPP10, ELMO1, EPDR1, FAM19A2, FLJ45983, FOSL1, FOXB1, GREM1, HMHA1, HOXA9, IFFO1, INPP4B, ITGB2, ITGB4, ITPKB, KCNIP2, KLHDC7B, LAT, LHX6, LIMK1, LOC100128239, LOC100192379, LOC646278, MAP2K2, MAX.chr1.210426156-210426257, MAX.chr1.84326495-84326656, MAX.chr10.119312785-119312882, MAX.chr15.67326025-67326060, MAX.chr16.54316401-54316453, MAX.chr16.85482306-85482494, MAX.chr17.74994454-74994572, MAX.chr17.76339840-76339972, MAX.chr2.7571082-7571136, MAX.chr21.45577347-45577679, MAX.chr3.14852538-14852568, MAX.chr3.187676564-187676668, MAX.chr4.174430662-174430793, MAX.chr5.177411809-177411836, MAX.chr6.45631561-45631625, MAX.chr7.25892382-25892451, MAX.chr7.402563-402641, MAX.chr7.64349554-64349606, MAX.chr8.142046239-142046398, MAX.chr8.145900842-145901246, MAX.chr9.126101804-126101848, MAX.chr9.126978999-126979182, MAX.chr9.36458633-36458725, MAX.chr9.87905315-87905326, MBP, MFNG, MT1A, MT1IP, NCOR2, NFATC1, NKX3-2, NRN1, OLIG1, PALLD, PAPLN, PDLIM2, PKN1, PRDM14, PRKG1, PRMT7, PTGER2, PTK2B, RAD52, RBM38, RHOF, RNF220, RTN4RL1, RXRA, SDCCAG8, SHROOM1, SKI, SLC12A8, SLC25A47, SPEG, SUCLG2, TBC1D10C, TMEM132E, VIPR2, WDR66, WNT6, ZDHHC18, ZNF382, and ZNF626 (Table 7).

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue controls). In accordance with these embodiments, the novel DMR(s) is from a gene selected from ALX4, Clorf114, CA8, CCNA1, CLSTN2, CR1, DAB1, DOK1, EMBP1, EPDR1, FLJ43390, FMN2, GDF6, GFRA1, HOXB3, LDLRAD2, LOC648809, MAPRE2, MAX.chr1.241587339-241587784, MAX.chr1.50798781-50799423, MAX.chr13.28527984-28528214, MAX.chr16.3221176-3221223, MAX.chr19.11805263-11805639, MAX.chr19.22034646-22034887, MAX.chr19.30718373-30719719, MAX.chr2.173099712-173099791, MAX.chr2.66808635-66808731, MAX.chr6.38683091-38683226, MAX.chr9.79638034-79638077, MECOM, ONECUT2, PARP15, SGIP1, SIM2, SORCS3, ST6GALNAC5, ST8SIA5, TFAP2E, TLX2, TLX3, VSTM2B, WDR17, ZNF254, ZNF43, ZNF491, ZNF763, and ZNF844 (Table 8).

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal buffy coat controls). In accordance with these embodiments, the novel DMR(s) is from a gene selected from FAM19A2, IFFO1, ITGB4, LOC100192379, MAX.chr1.84326495-84326656, MAX.chr16.85482306-85482494, MAX.chr6.45631561-45631625, MAX.chr7.25892382-25892451, MT1IP, NCOR2, OLIG1, RAD52, SHROOM1, SLC12A8, and TBC1D10C (Table 8).

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue or normal buffy coat controls). In accordance with these embodiments, the novel DMR(s) is from a gene selected from MAX.chr19.30718373-30719719, ITGB4, MAX.chr7.25892382-25892451, RAD52, SHROOM1, SLC12A8, and TBC1D10C (Table 8).

10131| Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue or normal buffy coat controls). In accordance with these embodiments, the novel DMR(s) is from a gene selected from ALX4, Clorf114, CA8, CCNA1, CLSTN2, CR1, DAB1, DOK1, EMBP1, EPDR1, FAM19A2, FLJ43390, FMN2, GDF6, GFRA1, HOXB3, IFFO1, ITGB4, LDLRAD2, LOC100192379, LOC648809, MAPRE2, MAX.chr1.241587339-241587784, MAX.chr1.50798781-50799423, MAX.chr1.84326495-84326656, MAX.chr13.28527984-28528214, MAX.chr16.3221176-3221223, MAX.chr16.85482306-85482494, MAX.chr19.11805263-11805639, MAX.chr19.22034646-22034887, MAX.chr19.30718373-30719719, MAX.chr2.173099712-173099791, MAX.chr2.66808635-66808731, MAX.chr6.38683091-38683226, MAX.chr6.45631561-45631625, MAX.chr7.25892382-25892451, MAX.chr9.79638034-79638077, MECOM, MT1IP, NCOR2, OLIG1, ONECUT2, PARP15, RAD52, SGIP1, SHROOM1, SIM2, SLC12A8, SORCS3, ST6GALNAC5, ST8SIA5, TBC1D10C, TFAP2E, TLX2, TLX3, VSTM2B, WDR17, ZNF254, ZNF43, ZNF491, ZNF763, and ZNF844 (Table 9).

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue or normal buffy coat controls). In accordance with these embodiments, the novel DMR(s) is from a gene selected from CA8, EMBP1, HOXB3, IFFO1, ITGB4, LOC100192379, LOC648809, MAX.chr1.84326495-84326656, MAX.chr16.3221176-3221223, MAX.chr16.85482306-85482494, MAX.chr19.30718373-30719719, MAX.chr9.79638034-79638077, MT1IP, ONECUT2, SHROOM1, SIM2, SLC12A8, TLX3, and ZNF763 (Table 9).

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue or normal buffy coat controls). In accordance with these embodiments, the novel DMR(s) is from a gene selected from Clorf114, CA8, CCNA1, EMBP1, EPDR1, FAM19A2, FMN2, HOXB3, IFFO1, ITGB4, LDLRAD2, LOC100192379, LOC648809, MAPRE2, MAX.chr1.50798781-50799423, MAX.chr1.84326495-84326656, MAX.chr16.3221176-3221223, MAX.chr19.11805263-11805639, MAX.chr2.66808635-66808731, MAX.chr6.38683091-38683226, MAX.chr6.45631561-45631625, MAX.chr9.79638034-79638077, MECOM, MT1IP, ONECUT2, PARP15, SHROOM1, SIM2, SLC12A8, SORCS3, ST6GALNAC5, ST8SIA5, TBC1D10C, TLX3, ZNF254, ZNF491, ZNF763, and ZNF844 (Table 10).

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) from control or benign tissue (e.g., normal tissue or normal buffy coat controls). In accordance with these embodiments, the novel DMR(s) is from a gene selected from CA8, EMBP1, HOXB3, IFFO1, ITGB4, LOC100192379, LOC648809, MAX.chr1.84326495-84326656, MAX.chr16.3221176-3221223, MAX.chr9.79638034-79638077, MT1IP, ONECUT2, SHROOM1, SIM2, SLC12A8, TLX3, and ZNF763 (Table 10).

Embodiments of the present disclosure also include novel differentially methylated regions (DMRs), each individually capable of distinguishing an oropharyngeal cancer (e.g., HPV⁺ oropharyngeal squamous cell cancer (HPV⁺ OPSCC)) in a saliva sample from a subject from control or benign tissue (e.g., saliva control samples). In accordance with these embodiments, the novel DMR(s) is from a gene selected from TLX3, MAX.chr16.3221176-3221223, TBC1D10C, and SHROOM1 (Table 11).

As described in the forgoing Examples, experiments were conducted to identify DMRs, also referred to herein as methylated DNA markers (MDMs), capable of distinguishing types and subtypes of oropharyngeal cancer from controls (e.g., healthy samples, benign samples, etc.). These experiments involved a validation study of the utility and performance of a panel of methylated DNA markers for detecting one more types or subtypes of oropharyngeal cancer by testing an independent set of case/control samples with a refined panel of markers. Such experiments resulted in the identification of MDMs useful for simultaneously detecting the presence of one or more types of oropharyngeal cancer (e.g., HPV⁺ oropharyngeal squamous cell cancer (HPV⁺ OPSCC)) from a control sample. A control sample can be a sample from a subject that does not have cancer, a sample from a subject that does not have an oropharyngeal cancer, a sample from a subject that has a type of cancer that is not an oropharyngeal cancer, or a sample from a subject that has an HPV(+) cancer that is not an oropharyngeal cancer. In some embodiments, the control sample is from a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and a stool sample. In some embodiments, the control sample is from an oropharyngeal tissue sample comprising one or more of soft palate cells or tissues, throat cells or tissues, tongue cells or tissues, and tonsil cells or tissues. In some embodiments, the tissue sample is an HPV(+) tissue sample.

In some embodiments, the present disclosure provides compositions and methods for identifying, determining, and/or classifying one or more types of oropharyngeal cancer from a biological sample (e.g., a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample). The methods generally comprise determining the methylation profile of at least one methylation marker in a biological sample isolated from a subject. In some embodiments, a change in the methylation state or profile of the marker is indicative of the presence, class, or site of a specific type of oropharyngeal cancer. Generally, such methods are useful for the detection of the presence or absence of specific types or subtypes of oropharyngeal cancer. In some embodiments, the types and subtypes of oropharyngeal cancer include, but are not limited to, HPV⁺ oropharyngeal squamous cell cancer (HPV⁺ OPSCC).

In some embodiments, methods are provided that comprise contacting a nucleic acid (e.g., genomic DNA) in a biological sample obtained from a subject with at least one reagent or series of reagents that distinguishes between methylated and non-methylated nucleotides (e.g., CpG dinucleotides) within at least one methylation marker; and detecting for the presence or absence of one or more types or subtypes of oropharyngeal cancer (e.g., afforded with a sensitivity of greater than or equal to 80% and a specificity of greater than or equal to 80%).

In some embodiments, methods are provided that comprise measuring a methylation level for one or more genes or methylated DNA markers in a biological sample from a human individual through treating genomic DNA in the biological sample with a reagent that modifies DNA in a methylation-specific manner; amplifying the treated genomic DNA using a set of primers for the selected one or more genes or methylation markers; and determining the methylation level of the one or more genes or methylation markers.

In some embodiments, methods are provided that comprise measuring an amount of one or more methylated DNA markers or genes in DNA from a biological sample; measuring an amount of at least one reference marker in the DNA; and calculating a value for the amount of the at least one methylated marker gene measured in the DNA as a percentage of the amount of the reference marker gene measured in the DNA, wherein the value indicates the amount of the at least one methylated marker DNA measured in the biological sample.

In some embodiments, methods are provided that comprise measuring a methylation level of a CpG site for one or more genes in a biological sample of a human individual through treating genomic DNA in the biological sample with bisulfite a reagent capable of modifying DNA in a methylation-specific manner; amplifying the modified genomic DNA using a set of primers for the selected one or more genes; and determining the methylation level of the CpG site for the selected one or more genes.

In some embodiments, the present disclosure provides methods for characterizing a biological sample comprising measuring one or both of a methylation level of a CpG site for one or more genes in a biological sample of a human individual through treating genomic DNA in the biological sample with bisulfite; amplifying the bisulfite-treated genomic DNA using a set of primers for the selected one or more genes; and determining the methylation level of the CpG site. In some embodiments, the method comprises comparing one or both of the methylation level of a methylation marker to a methylation level of a corresponding set of genes in control samples without a specific type of cancer; and/or determining that a subject has one or more types or subtypes of oropharyngeal cancer when one or both of the methylation level measured in the one or more genes is higher than the methylation level measured in the respective control samples.

In some embodiments, the present disclosure provides methods of measuring in a biological sample a methylation level of one or more genes or markers through treating genomic DNA in the biological sample with bisulfite; amplifying the bisulfite-treated genomic DNA using a set of primers for the selected one or more genes; and determining the methylation level of the one or more genes or markers.

In some embodiments, the present disclosure provides methods of screening for one or more types or subtypes of oropharyngeal cancer in a sample obtained from a subject. In accordance with these embodiments, the method includes assaying a methylation state or profile of one or more methylated DNA markers; and identifying the subject as having one or more types or subtypes of oropharyngeal cancer when the methylation state or profile of the marker is different than a methylation state or profile of the marker assayed in a subject that does not have the one or more types of cancer.

In some embodiments, the present disclosure provides methods that comprise measuring a methylation level for one or more genes or markers in a biological sample of a human individual through treating genomic DNA in the biological sample with a reagent that modifies DNA in a methylation-specific manner; amplifying the treated genomic DNA using a set of primers for the selected one or more genes or markers; and determining the methylation level of the one or more genes or markers.

In some embodiments, the present disclosure provides methods for characterizing a biological sample comprising measuring an amount of at least one methylated DNA marker in DNA extracted from the biological sample; treating genomic DNA in the biological sample with bisulfite; amplifying the bisulfite-treated genomic DNA using primers specific for a CpG site for each marker, wherein the primers specific for each marker are capable of binding an amplicon bound by a primer sequence for the marker recited in Tables 3 and 12, wherein the amplicon bound by the primer sequence for the marker recited in Tables 3 and 12 is at least a portion of a genetic region for a methylated marker recited in Tables 1, 2, 6, or 7; and determining the methylation level of the CpG site for one or more genes.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of oropharyngeal cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Tables 1, 2, 6, or 7; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of oropharyngeal cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 1; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of oropharyngeal cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 2; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of oropharyngeal cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 6; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having one or more types or subtypes of oropharyngeal cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 7; and measuring the methylation level of one or more methylated markers.

In some embodiments, the present disclosure provides methods comprising extracting genomic DNA from a biological sample of a human individual suspected of having or having cancer, treating the extracted genomic DNA with bisulfite, amplifying the bisulfite-treated genomic DNA using separate primers specific for CpG sites for one or more of the methylated DNA markers, and measuring a methylation level of the CpG site for each of the one or more markers.

In some embodiments, the present disclosure provides methods for preparing a DNA fraction from a biological sample of a human individual useful for analyzing one or more genetic loci involved in one or more chromosomal aberrations. In accordance with these embodiments, the method comprises extracting genomic DNA from a biological sample of a human individual; producing a fraction of the extracted genomic DNA by treating the extracted genomic DNA with a reagent that modifies DNA in a methylation-specific manner; amplifying the bisulfite-treated genomic DNA using separate primers specific for one or more methylated DNA markers; analyzing one or more genetic loci in the produced fraction of the extracted genomic DNA by measuring a methylation level of the CpG site for each of the one or more markers.

In some embodiments, the present disclosure provides methods for preparing a DNA fraction from a biological sample of a human individual useful for analyzing one or more DNA fragments involved in one or more chromosomal aberrations. In accordance with these embodiments, the method comprises extracting genomic DNA from a biological sample of a human individual; producing a fraction of the extracted genomic DNA by treating the extracted genomic DNA with a reagent that modifies DNA in a methylation-specific manner; amplifying the bisulfite-treated genomic DNA using separate primers specific for one or more methylated DNA markers; and analyzing one or more DNA fragments in the produced fraction of the extracted genomic DNA by measuring a methylation level of the CpG site for each of the one or more markers.

As would be appreciated by one of ordinary skill in the art based on the present disclosure, the various methods described herein are not limited to the use of any one specific methylated DNA markers, methylated marker genes, methylated genes, and/or DMRs. That is, one or more of the methylated DNA markers, methylated marker genes, methylated genes, and/or DMRs of the present disclosure can be used to distinguish and/or identify one or more types or subtypes of an oropharyngeal cancer, including any combinations thereof. Additionally, the methylated DNA markers, methylated marker genes, methylated genes, and/or DMRs of the present disclosure can comprise a region or subregion (e.g., a gene on a chromosome, a single nucleotide, a CpG island, etc.) of any of the markers listed in Tables 1, 2, 6 and 7.

In some embodiments, the DMR is from a gene selected from ABCB1, ARHGAP12, ASCL1, Clorf114, EMX1, GRIN2D, LOC645323, MAX.chr6.58147682-58147771, MAX.chr9.36739811-36739868, NEUROG3, NID2, TBX15, TMEM200C, TSPYL5, TTYH1, VWC2, ZNF610, ZNF69, ZNF773, ZNF781, ALX4, ATP10A, C1QL3, CA8, CACNA1A, CACNG8, CALCA, CCNA1, CLIC6, CLSTN2, CR1, CTNND2, DAB1, DGKG, DOK1, DOK6, DPP4, DUXA, ELMO1, EMBP1, EPDR1, FGF12, FLJ43390, FMN2, FOXB2, FOXD4, FREM3, GALR1, GDF6, GFRA1, GRIK3, HOXB3, HOXB4, HPSE2, LDLRAD2, LHX2, LOC100131366, LOC345643, LOC386758, LOC648809, LOC728392, MAML3, MAPRE2, MAX.chr1.226288154-226288189, MAX.chr1.2375078-2375126, MAX.chr1.241587339-241587784, MAX.chr1.50798781-50799423, MAX.chr10.22765150-22765477, MAX.chr10.23462342-23462436, MAX.chr11.14926602-14927044, MAX.chr11.58903531-58903592, MAX.chr13.28527984-28528214, MAX.chr13.29106641-29107037, MAX.chr14.100784488-100784782, MAX.chr16.3221176-3221223, MAX.chr16.3222040-3222098, MAX.chr16.71460171-71460282, MAX.chr19.11805263-11805639, MAX.chr19.16394457-16394646, MAX.chr19.21657626-21657769, MAX.chr19.22034646-22034887, MAX.chr19.23299989-23300156, MAX.chr19.30713427-30713588, MAX.chr19.30716926-30717074, MAX.chr19.30718373-30719719, MAX.chr2.118981724-118982174, MAX.chr2.127783107-127783403, MAX.chr2.173099712-173099791, MAX.chr2.66808635-66808731, MAX.chr22.50064113-50064259, MAX.chr3.137489884-137490061, MAX.chr5.138923141-138923219, MAX.chr5.42995180-42995535, MAX.chr6.38683091-38683226, MAX.chr7.121952014-121952084, MAX.chr7.155166980-155167310, MAX.chr8.99986792-99986864, MAX.chr9.79627078-79627116, MAX.chr9.79638034-79638077, MAX.chr9.98789824-98789847, MDFI, MECOM, MED12L, MIR129-2, MIR196A1, NELL1, NPY, ONECUT2, OPCML, PARP15, PDGFD, PEX5L, PRR15, SEMA6A, SFMBT2, SGIP1, SIM2, SLC35F3, SLCO4C1, SORCS3, ST6GALNAC5, ST8SIA5, SV2C, TACC2, TFAP2E, TLX2, TLX3, TRH, TRIM58, VAV3, VSTM2B, WDR17, ZNF254, ZNF43, ZNF486, ZNF491, ZNF518B, ZNF542, ZNF625, ZNF665, ZNF671, ZNF763, ZNF844, AGRN, ANKRD35, ARHGAP27, ARHGAP30, BCL2L11, BIN2, C10orf114, C4orf31, C6orf132, C6orf186, CCDC88B, CRHBP, DAPK1, DNMT3A, DPP10, FAM19A2, FLJ45983, FOSL1, FOXB1, GREM1, HMHA1, HOXA9, IFFO1, INPP4B, ITGB2, ITGB4, ITPKB, KCNIP2, KLHDC7B, LAT, LHX6, LIMK1, LOC100128239, LOC100192379, LOC646278, MAP2K2, MAX.chr1.210426156-210426257, MAX.chr1.84326495-84326656, MAX.chr10.119312785-119312882, MAX.chr15.67326025-67326060, MAX.chr16.54316401-54316453, MAX.chr16.85482306-85482494, MAX.chr17.74994454-74994572, MAX.chr17.76339840-76339972, MAX.chr2.7571082-7571136, MAX.chr21.45577347-45577679, MAX.chr3.14852538-14852568, MAX.chr3.187676564-187676668, MAX.chr4.174430662-174430793, MAX.chr5.177411809-177411836, MAX.chr6.45631561-45631625, MAX.chr7.25892382-25892451, MAX.chr7.402563-402641, MAX.chr7.64349554-64349606, MAX.chr8.142046239-142046398, MAX.chr8.145900842-145901246, MAX.chr9.126101804-126101848, MAX.chr9.126978999-126979182, MAX.chr9.36458633-36458725, MAX.chr9.87905315-87905326, MBP, MENG, MT1A, MT1IP, NCOR2, NFATC1, NKX3-2, NRN1, OLIG1, PALLD, PAPLN, PDLIM2, PKN1, PRDM14, PRKG1, PRMT7, PTGER2, PTK2B, RAD52, RBM38, RHOF, RNF220, RTN4RL1, RXRA, SDCCAG8, SHROOM1, SKI, SLC12A8, SLC25A47, SPEG, SUCLG2, TBC1D10C, TMEM132E, VIPR2, WDR66, WNT6, ZDHHC18, ZNF382, and ZNF626 (Table 1); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from ABCB1, ARHGAP12, ASCL1, Clorf114, EMX1, GRIN2D, LOC645323, MAX.chr6.58147682-58147771, MAX.chr9.36739811-36739868, NEUROG3, NID2, TBX15, TMEM200C, TSPYL5, TTYH1, VWC2, ZNF610, ZNF69, ZNF773, and ZNF781 (Table 2); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from ALX4, ATP10A, Clorf114, C1QL3, CA8, CACNA1A, CACNG8, CALCA, CCNA1, CLIC6, CLSTN2, CR1, CTNND2, DAB1, DGKG, DOK1, DOK6, DPP4, DUXA, ELMO1, EMBP1, EPDR1, FGF12, FLJ43390, FMN2, FOXB2, FOXD4, FREM3, GALR1, GDF6, GFRA1, GRIK3, HOXB3, HOXB4, HPSE2, LDLRAD2, LHX2, LOC100131366, LOC345643, LOC386758, LOC645323, LOC648809, LOC728392, MAML3, MAPRE2, MAX.chr1.226288154-226288189, MAX.chr1.2375078-2375126, MAX.chr1.241587339-241587784, MAX.chr1.50798781-50799423, MAX.chr10.22765150-22765477, MAX.chr10.23462342-23462436, MAX.chr11.14926602-14927044, MAX.chr11.58903531-58903592, MAX.chr13.28527984-28528214, MAX.chr13.29106641-29107037, MAX.chr14.100784488-100784782, MAX.chr16.3221176-3221223, MAX.chr16.3222040-3222098, MAX.chr16.71460171-71460282, MAX.chr19.11805263-11805639, MAX.chr19.16394457-16394646, MAX.chr19.21657626-21657769, MAX.chr19.22034646-22034887, MAX.chr19.23299989-23300156, MAX.chr19.30713427-30713588, MAX.chr19.30716926-30717074, MAX.chr19.30718373-30719719, MAX.chr2.118981724-118982174, MAX.chr2.127783107-127783403, MAX.chr2.173099712-173099791, MAX.chr2.66808635-66808731, MAX.chr22.50064113-50064259, MAX.chr3.137489884-137490061, MAX.chr5.138923141-138923219, MAX.chr5.42995180-42995535, MAX.chr6.38683091-38683226, MAX.chr7.121952014-121952084, MAX.chr7.155166980-155167310, MAX.chr8.99986792-99986864, MAX.chr9.79627078-79627116, MAX.chr9.79638034-79638077, MAX.chr9.98789824-98789847, MDFI, MECOM, MED12L, MIR129-2, MIR196A1, NELL1, NPY, ONECUT2, OPCML, PARP15, PDGFD, PEX5L, PRR15, SEMA6A, SFMBT2, SGIP1, SIM2, SLC35F3, SLCO4C1, SORCS3, ST6GALNAC5, ST8SIA5, SV2C, TACC2, TFAP2E, TLX2, TLX3, TRH, TRIM58, VAV3, VSTM2B, WDR17, ZNF254, ZNF43, ZNF486, ZNF491, ZNF518B, ZNF542, ZNF625, ZNF665, ZNF671, ZNF763, and ZNF844 (Table 6); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from AGRN, ANKRD35, ARHGAP27, ARHGAP30, BCL2L11, BIN2, C10orf114, C4orf31, C6orf132, C6orf186, CCDC88B, CRHBP, DAPK1, DNMT3A, DPP10, ELMO1, EPDR1, FAM19A2, FLJ45983, FOSL1, FOXB1, GREM1, HMHA1, HOXA9, IFFO1, INPP4B, ITGB2, ITGB4, ITPKB, KCNIP2, KLHDC7B, LAT, LHX6, LIMK1, LOC100128239, LOC100192379, LOC646278, MAP2K2, MAX.chr1.210426156-210426257, MAX.chr1.84326495-84326656, MAX.chr10.119312785-119312882, MAX.chr15.67326025-67326060, MAX.chr16.54316401-54316453, MAX.chr16.85482306-85482494, MAX.chr17.74994454-74994572, MAX.chr17.76339840-76339972, MAX.chr2.7571082-7571136, MAX.chr21.45577347-45577679, MAX.chr3.14852538-14852568, MAX.chr3.187676564-187676668, MAX.chr4.174430662-174430793, MAX.chr5.177411809-177411836, MAX.chr6.45631561-45631625, MAX.chr7.25892382-25892451, MAX.chr7.402563-402641, MAX.chr7.64349554-64349606, MAX.chr8.142046239-142046398, MAX.chr8.145900842-145901246, MAX.chr9.126101804-126101848, MAX.chr9.126978999-126979182, MAX.chr9.36458633-36458725, MAX.chr9.87905315-87905326, MBP, MENG, MT1A, MT1IP, NCOR2, NFATC1, NKX3-2, NRN1, OLIG1, PALLD, PAPLN, PDLIM2, PKN1, PRDM14, PRKG1, PRMT7, PTGER2, PTK2B, RAD52, RBM38, RHOF, RNF220, RTN4RL1, RXRA, SDCCAG8, SHROOM1, SKI, SLC12A8, SLC25A47, SPEG, SUCLG2, TBC1D10C, TMEM132E, VIPR2, WDR66, WNT6, ZDHHC18, ZNF382, and ZNF626 (Table 7); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from ALX4, Clorf114, CA8, CCNA1, CLSTN2, CR1, DAB1, DOK1, EMBP1, EPDR1, FLJ43390, FMN2, GDF6, GFRA1, HOXB3, LDLRAD2, LOC648809, MAPRE2, MAX.chr1.241587339-241587784, MAX.chr1.50798781-50799423, MAX.chr13.28527984-28528214, MAX.chr16.3221176-3221223, MAX.chr19.11805263-11805639, MAX.chr19.22034646-22034887, MAX.chr19.30718373-30719719, MAX.chr2.173099712-173099791, MAX.chr2.66808635-66808731, MAX.chr6.38683091-38683226, MAX.chr9.79638034-79638077, MECOM, ONECUT2, PARP15, SGIP1, SIM2, SORCS3, ST6GALNAC5, ST8SIA5, TFAP2E, TLX2, TLX3, VSTM2B, WDR17, ZNF254, ZNF43, ZNF491, ZNF763, and ZNF844 (Table 8); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from FAM19A2, IFFO1, ITGB4, LOC100192379, MAX.chr1.84326495-84326656, MAX.chr16.85482306-85482494, MAX.chr6.45631561-45631625, MAX.chr7.25892382-25892451, MT1IP, NCOR2, OLIG1, RAD52, SHROOM1, SLC12A8, and TBC1D10C (Table 8); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from MAX.chr19.30718373-30719719, ITGB4, MAX.chr7.25892382-25892451, RAD52, SHROOM1, SLC12A8, and TBC1D10C (Table 8); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from ALX4, Clorf114, CA8, CCNA1, CLSTN2, CR1, DAB1, DOK1, EMBP1, EPDR1, FAM19A2, FLJ43390, FMN2, GDF6, GFRA1, HOXB3, IFFO1, ITGB4, LDLRAD2, LOC100192379, LOC648809, MAPRE2, MAX.chr1.241587339-241587784, MAX.chr1.50798781-50799423, MAX.chr1.84326495-84326656, MAX.chr13.28527984-28528214, MAX.chr16.3221176-3221223, MAX.chr16.85482306-85482494, MAX.chr19.11805263-11805639, MAX.chr19.22034646-22034887, MAX.chr19.30718373-30719719, MAX.chr2.173099712-173099791, MAX.chr2.66808635-66808731, MAX.chr6.38683091-38683226, MAX.chr6.45631561-45631625, MAX.chr7.25892382-25892451, MAX.chr9.79638034-79638077, MECOM, MT1IP, NCOR2, OLIG1, ONECUT2, PARP15, RAD52, SGIP1, SHROOM1, SIM2, SLC12A8, SORCS3, ST6GALNAC5, ST8SIA5, TBC1D10C, TFAP2E, TLX2, TLX3, VSTM2B, WDR17, ZNF254, ZNF43, ZNF491, ZNF763, and ZNF844 (Table 9); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from CA8, EMBP1, HOXB3, IFFO1, ITGB4, LOC100192379, LOC648809, MAX.chr1.84326495-84326656, MAX.chr16.3221176-3221223, MAX.chr16.85482306-85482494, MAX.chr19.30718373-30719719, MAX.chr9.79638034-79638077, MT1IP, ONECUT2, SHROOM1, SIM2, SLC12A8, TLX3, and ZNF763 (Table 9); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from Clorf114, CA8, CCNA1, EMBP1, EPDR1, FAM19A2, FMN2, HOXB3, IFFO1, ITGB4, LDLRAD2, LOC100192379, LOC648809, MAPRE2, MAX.chr1.50798781-50799423, MAX.chr1.84326495-84326656, MAX.chr16.3221176-3221223, MAX.chr19.11805263-11805639, MAX.chr2.66808635-66808731, MAX.chr6.38683091-38683226, MAX.chr6.45631561-45631625, MAX.chr9.79638034-79638077, MECOM, MT1IP, ONECUT2, PARP15, SHROOM1, SIM2, SLC12A8, SORCS3, ST6GALNAC5, ST8SIA5, TBC1D10C, TLX3, ZNF254, ZNF491, ZNF763, and ZNF844 (Table 10); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from CA8, EMBP1, HOXB3, IFFO1, ITGB4, LOC100192379, LOC648809, MAX.chr1.84326495-84326656, MAX.chr16.3221176-3221223, MAX.chr9.79638034-79638077, MT1IP, ONECUT2, SHROOM1, SIM2, SLC12A8, TLX3, and ZNF763 (Table 10); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., control oropharyngeal tissue or control buffy coat sample).

In some embodiments, the DMR is from a gene selected from TLX3, MAX.chr16.3221176-3221223, TBC1D10C, and SHROOM1 (Table 11); and the subject has or is suspected of having oropharyngeal cancer (e.g., oropharyngeal squamous cell carcinoma (HPV(+)OPSCC)). In some embodiments, determining the methylation profile of the DMR comprises comparing the methylation profile to a corresponding region from a control DNA sample (e.g., saliva sample).

In some embodiments, the DMR(s) capable of distinguishing oropharyngeal cancer from a control sample is associated with an area under a ROC curve (AUC) greater than or equal to 0.5, wherein the ROC curve discriminates between a subject having or suspected of having OPSCC and a control DNA sample. In some embodiments, the DMR(s) capable of distinguishing oropharyngeal cancer from a control sample is associated with an area under a ROC curve (AUC) greater than or equal to 0.6, wherein the ROC curve discriminates between a subject having or suspected of having OPSCC and a control DNA sample. In some embodiments, the DMR(s) capable of distinguishing oropharyngeal cancer from a control sample is associated with an area under a ROC curve (AUC) greater than or equal to 0.7, wherein the ROC curve discriminates between a subject having or suspected of having OPSCC and a control DNA sample. In some embodiments, the DMR(s) capable of distinguishing oropharyngeal cancer from a control sample is associated with an area under a ROC curve (AUC) greater than or equal to 0.8, wherein the ROC curve discriminates between a subject having or suspected of having OPSCC and a control DNA sample. In some embodiments, the DMR(s) capable of distinguishing oropharyngeal cancer from a control sample is associated with an area under a ROC curve (AUC) greater than or equal to 0.9, wherein the ROC curve discriminates between a subject having or suspected of having OPSCC and a control DNA sample.

In some embodiments, the DMR(s) capable of distinguishing oropharyngeal cancer from a control sample comprises an increased methylation percentage as compared to a control DNA sample. In some embodiments, the DMR(s) capable of distinguishing oropharyngeal cancer from a control sample comprises an increased hypermethylation ratio as compared to a control DNA sample.

In some embodiments, determining the methylation profile of at least one DMR comprises amplifying at least a portion of the DMR using a set of primers (e.g., Tables 3, and 12). In some embodiments, determining the methylation profile of at least one DMR comprises performing at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR. In some embodiments, determining the methylation profile of at least one DMR comprises determining the presence or absence of methylation at a CpG site. In some embodiments, the one or more CpG sites are present in a coding region, a non-coding region, and/or a regulatory region of a gene (e.g., any one of the genes disclosed herein). In some embodiments, the DMR(s) capable of distinguishing an oropharyngeal cancer from a control sample can be validated using at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR. In some embodiments, the DMR(s) capable of distinguishing an oropharyngeal cancer from a control sample can be assessed based on at least one of an area under a ROC curve (AUC), fold-change in methylation, methylation percentage, and/or hypermethylation ratio between a test sample and a control sample.

As one of ordinary skill in the art would understand based on the present disclosure, one or more types or subtypes of oropharyngeal cancers can be predicted by various combinations of markers (e.g., as identified by statistical techniques related to specificity and sensitivity of prediction). Embodiments of the present disclosure provide methods for identifying predictive combinations and validated predictive combinations for one or more types or subtypes of oropharyngeal cancers.

Such methods are not limited to a subject type. In some embodiments, the subject is a mammal. In some embodiments, the subject is a human. Such methods are not limited to a particular manner or technique for measuring protein expression and/or activity. Techniques for measuring protein expression and/or activity levels are known in the art. Indeed, any known technique for measuring protein expression and/or activity levels are contemplated and herein incorporated.

Such methods are not limited to a particular manner or technique for determining characterizing, measuring, or assaying methylation for one or more methylated markers, methylated marker genes, genes, DMRs, and/or DNA methylated markers. In some embodiments, such techniques are based upon an analysis of the methylation status (e.g., CpG methylation status) of at least one marker, region of a marker, or base of a marker comprising a DMR.

In some embodiments, measuring the methylation state or profile of a methylated DNA marker in a sample comprises determining the methylation state of one nucleotide base. In some embodiments, measuring the methylation state of a methylated DNA marker in the sample comprises determining the extent of methylation at a plurality of nucleotide bases. Moreover, in some embodiments, the methylation state or profile of a methylated DNA marker comprises an increase in methylation of the marker relative to a normal methylation state or profile of the marker. In some embodiments, the methylation state or profile of the marker comprises decreased methylation of the marker relative to a normal methylation state of the marker. In some embodiments the methylation state or profile of the marker comprises a different pattern of methylation of the marker relative to a normal methylation state or profile of the marker.

Furthermore, in some embodiments the marker is a region of 100 or fewer nucleotide bases. In some embodiments, the marker is a region of 500 or fewer nucleotide bases. In some embodiments, the marker is a region of 1000 or fewer nucleotide bases. In some embodiments, the marker is a region of 5000 or fewer nucleotide bases. In some embodiments, the marker is one nucleotide base. In some embodiments, the marker is in a high CpG density promoter region.

In certain embodiments, methods for analyzing a nucleic acid for the presence of 5-methylcytosine involves treatment of DNA with a reagent that modifies DNA in a methylation-specific manner. Examples of such reagents include, but are not limited to, a methylation-sensitive restriction enzyme, a methylation-dependent restriction enzyme, a bisulfite reagent, a TET enzyme, and a borane reducing agent.

A frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98), methylation-specific PCR (MSP) as is disclosed, e.g., in U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; and in U.S. Pat. Nos. 8,361,720; 8,715,937; 8,916,344; and 9,212,392.

In some embodiments, conventional techniques include methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then assaying the product by sequencing (Olek & Walter (1997) Nat. Genet. 17: 275-6) or using a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures can be used in conjunction with bisulfite treatment according to embodiments of the present disclosure. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-specific restriction enzymes, e.g., methylation-sensitive or methylation-dependent enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Hornsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, DMR, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include DNA denaturation buffer; sulfonation buffer; DNA recovery reagents or kits (e.g., precipitation, ultrafiltration, affinity column); desulfonation buffer; and DNA recovery components.

Assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase.

MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to methylated and unmethylated PCR primers for specific loci (e.g., specific genes, markers, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g., a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can be used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QuARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophores per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

In some embodiments, fragments of the treated DNA are amplified using sets of primer oligonucleotides (e.g., see Tables 3 and 12) and an amplification enzyme, according to the method and compositions described herein. The amplification of several DNA segments can be carried out simultaneously in one and the same reaction vessel. Typically, the amplification is carried out using a polymerase chain reaction (PCR). Amplicons are typically 100 to 2000 base pairs in length.

In some embodiments of the method, the methylation status or profile of CpG positions within or near a differentially methylated region (e.g., Tables 1, 2, 6, and 7) may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG.

Such methods are not limited to a specific type or kind of primer or primer pair related to the one or more methylated markers, methylated marker genes, genes, DMRs, and/or methylated DNA markers. In some embodiments, the primer or primer pair is recited in Tables 3 and 12 (SEQ ID NOs: 1-176). In some embodiments, the primer or primer pair specific for each methylated marker gene are capable of binding an amplicon bound by a primer sequence for the marker gene recited in Tables 3 and 12, wherein the amplicon bound by the primer sequence for the marker gene recited in Tables 3 and 12 is at least a portion of a genetic region for the methylated marker gene recited in Tables 1, 2, 6, or 7. In some embodiments, the primer or primer pair for a methylated marker is a set of primers that specifically binds at least a portion of a genetic region comprising chromosomal coordinates for the specific methylated marker recited in Tables 1, 2, 6, or 7.

In another embodiment, the present disclosure provides a method for converting an oxidized 5-methylcytosine residue in cell-free DNA to a dihydrouracil residue (see, Liu et al., 2019, Nat Biotechnol. 37, pp. 424-429; U.S. Patent Application Publication No. 202000370114). The method involves reaction of an oxidized 5mC residue selected from 5-formylcytosine (5fC), 5-carboxymethylcytosine (5caC), and combinations thereof, with a borane reducing agent. The oxidized 5mC residue may be naturally occurring or, more typically, the result of a prior oxidation of a 5mC or 5hmC residue, e.g., oxidation of 5mC or 5hmC with a TET family enzyme (e.g., TET1, TET2, or TET3), or chemical oxidation of 5 mC or 5hmC, e.g., with potassium perruthenate (KRuO₄) or an inorganic peroxo compound or composition such as peroxotungstate (see, e.g., Okamoto et al. (2011) Chem. Commun. 47:11231-33) and a copper (II) perchlorate/2,2,6,6-tetramethylpiperidine-1-oxyl (TEMPO) combination (see Matsushita et al. (2017) Chem. Commun. 53:5756-59).

The borane reducing agent may be characterized as a complex of borane and a nitrogen-containing compound selected from nitrogen heterocycles and tertiary amines. The nitrogen heterocycle may be monocyclic, bicyclic, or polycyclic, but is typically monocyclic, in the form of a 5- or 6-membered ring that contains a nitrogen heteroatom and optionally one or more additional heteroatoms selected from N, O, and S. The nitrogen heterocycle may be aromatic or alicyclic. Preferred nitrogen heterocycles herein include 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,4-triazole, pyridazine, pyrimidine, pyrazine, 1,2,4-triazine, and 1,3,5-triazine, any of which may be unsubstituted or substituted with one or more non-hydrogen substituents. Typical non-hydrogen substituents are alkyl groups, particularly lower alkyl groups, such as methyl, ethyl, n-propyl, isopropyl, n-butyl, isobutyl, t-butyl, and the like. Exemplary compounds include pyridine borane, 2-methylpyridine borane (also referred to as 2-picoline borane), and 5-ethyl-2-pyridine.

The reaction of the borane reducing agent with the oxidized 5mC residue in cell-free DNA is advantageous insofar as non-toxic reagents and mild reaction conditions can be employed; there is no need for any bisulfate, nor for any other potentially DNA-degrading reagents. Furthermore, conversion of an oxidized 5mC residue to dihydrouracil with the borane reducing agent can be carried out without need for isolation of any intermediates, in a “one-pot” or “one-tube”reaction. This is quite significant, since the conversion involves multiple steps, i.e., (1) reduction of the alkene bond linking C-4 and C-5 in the oxidized 5mC, (2) deamination, and (3) either decarboxylation, if the oxidized 5mC is 5caC, or deformylation, if the oxidized 5mC is 5fC.

In addition to a method for converting an oxidized 5-methylcytosine residue in cell-free DNA to a dihydrouracil residue, the present disclosure also provides a reaction mixture related to the aforementioned method. The reaction mixture comprises a sample of cell-free DNA containing at least one oxidized 5-methylcytosine residue selected from 5caC, 5fC, and combinations thereof, and a borane reducing agent effective to effective to reduce, deaminate, and either decarboxylate or deformylate the at least one oxidized 5-methylcytosine residue. The borane reducing agent is a complex of borane and a nitrogen-containing compound selected from nitrogen heterocycles and tertiary amines, as explained above. In a preferred embodiment, the reaction mixture is substantially free of bisulfite, meaning substantially free of bisulfite ion and bisulfite salts. Ideally, the reaction mixture contains no bisulfite.

In a related aspect of the present disclosure, a kit is provided for converting 5mC residues in cell-free DNA to dihydrouracil residues, where the kit includes a reagent for blocking 5hmC residues, a reagent for oxidizing 5mC residues beyond hydroxymethylation to provide oxidized 5mC residues, and a borane reducing agent effective to reduce, deaminate, and either decarboxylate or deformylate the oxidized 5mC residues. The kit may also include instructions for using the components to carry out the above-described method.

In another embodiment, a method is provided that makes use of the above-described oxidation reaction. The method enables detecting the presence and location of 5-methylcytosine residues in cell-free DNA, and comprises the following steps: (a) modifying 5hmC residues in fragmented, adapter-ligated cell-free DNA to provide an affinity tag thereon, wherein the affinity tag enables removal of modified 5hmC-containing DNA from the cell-free DNA; (b) removing the modified 5hmC-containing DNA from the cell-free DNA, leaving DNA containing unmodified 5mC residues; (c) oxidizing the unmodified 5mC residues to give DNA containing oxidized 5mC residues selected from 5caC, 5fC, and combinations thereof; (d) contacting the DNA containing oxidized 5mC residues with a borane reducing agent effective to reduce, deaminate, and either decarboxylate or deformylate the oxidized 5mC residues, thereby providing DNA containing dihydrouracil residues in place of the oxidized 5mC residues; (e) amplifying and sequencing the DNA containing dihydrouracil residues; (f) determining a 5-methylation pattern from the sequencing results in (e).

In some embodiments, the present disclosure provides a method for identifying 5-methylcytosine (5mC) or 5-hydroxymethylcytosine (5hmC) in a target nucleic acid. In some embodiments, the method comprises providing a biological sample comprising the target nucleic acid, modifying the target nucleic acid by converting the 5mC and 5hmC in the nucleic acid sample to 5-carboxylcytosine (5caC) and/or 5-formylcytosine (5fC) by contacting the nucleic acid sample with a TET enzyme so that one or more 5caC or 5fC residues are generated, and converting the 5caC and/or 5fC to dihydrouracil (DHU) by treating the target nucleic acid with a borane reducing agent to provide a modified nucleic acid sample comprising a modified target nucleic acid, and detecting the sequence of the modified target nucleic acid; wherein a cytosine (C) to thymine (T) transition or a cytosine (C) to DHU transition in the sequence of the modified target nucleic acid compared to the target nucleic acid provides the location of either a 5mC or 5hmC in the target nucleic acid. In some embodiments, the borane reducing agent is 2-picoline borane.

In some embodiments, detecting the sequence of the modified target nucleic acid comprises one or more of chain termination sequencing, microarray, high-throughput sequencing, and restriction enzyme analysis. In some embodiments, the TET enzyme is selected from the group consisting of human TET1, TET2, and TET3; murine TET1, TET2, and TET3; Naegleria TET (NgTET); and Coprinopsis cinerea (CcTET). In some embodiments, the method further comprises a step of blocking one or more modified cytosines. In some embodiments, the step of blocking comprises adding a sugar to a 5hmC. In some embodiments, the method further comprises a step of amplifying the copy number of one or more nucleic acid sequences. In some embodiments, the oxidizing agent is potassium perruthenate or Cu (II)/TEMPO (2,2,6,6-tetramethylpiperidine-1-oxyl.)

The cell-free DNA is typically extracted from a biological sample from a subject, where the sample can be whole blood, plasma, urine, saliva, mucosal excretions, organ secretions, sputum, stool, or tears. In some embodiments, the cell-free DNA is derived from a tumor (e.g., an oropharyngeal tumor). In other embodiments, the cell-free DNA is from a patient with a disease or other pathogenic condition. The cell-free DNA may or may not be derived from a tumor. In some embodiments, the cell-free DNA in which 5hmC residues are to be modified is in purified, fragmented form, and adapter-ligated. DNA purification in this context can be carried out using any suitable method known to those of ordinary skill in the art and/or described in the pertinent literature, and, while cell-free DNA can itself be highly fragmented, further fragmentation may occasionally be desirable, as described, for example, in U.S. Patent Publication No. 2017/0253924. The cell-free DNA fragments are generally in the size range of about 20 nucleotides to about 500 nucleotides, more typically in the range of about 20 nucleotides to about 250 nucleotides. The purified cell-free DNA fragments that are modified in step (a) have been end-repaired using conventional means (e.g., a restriction enzyme) so that the fragments have a blunt end at each 3′ and 5′ terminus. In a preferred method, as described in WO 2017/176630, the blunted fragments have also been provided with a 3′ overhang comprising a single adenine residue using a polymerase such as Taq polymerase. This facilitates subsequent ligation of a selected universal adapter, i.e., an adapter such as a Y-adapter or a hairpin adapter that ligates to both ends of the cell-free DNA fragments and contains at least one molecular barcode. Use of adapters also enables selective PCR enrichment of adapter-ligated DNA fragments.

In some embodiments, the “purified, fragmented cell-free DNA” comprises adapter-ligated DNA fragments. Modification of 5hmC residues in these cell-free DNA fragments with an affinity tag is done so as to enable subsequent removal of the modified 5hmC-containing DNA from the cell-free DNA. In one embodiment, the affinity tag comprises a biotin moiety, such as biotin, desthiobiotin, oxybiotin, 2-iminobiotin, diaminobiotin, biotin sulfoxide, biocytin, or the like. Use of a biotin moiety as the affinity tag allows for facile removal with streptavidin (e.g., streptavidin beads, magnetic streptavidin beads, etc.).

Tagging 5hmC residues with a biotin moiety or other affinity tag is accomplished by covalent attachment of a chemoselective group to 5hmC residues in the DNA fragments, where the chemoselective group is capable of undergoing reaction with a functionalized affinity tag so as to link the affinity tag to the 5hmC residues. In one embodiment, the chemoselective group is UDP glucose-6-azide, which undergoes a spontaneous 1,3-cycloaddition reaction with an alkyne-functionalized biotin moiety, as described in Robertson et al. (2011) Biochem. Biophys. Res. Comm. 411(1):40-3, U.S. Pat. No. 8,741,567, and WO 2017/176630. Addition of an alkyne-functionalized biotin-moiety thus results in covalent attachment of the biotin moiety to each 5hmC residue.

The affinity-tagged DNA fragments can then be pulled down using, in one embodiment, streptavidin, in the form of streptavidin beads, magnetic streptavidin beads, or the like, and set aside for later analysis, if so desired. The supernatant remaining after removal of the affinity-tagged fragments contains DNA with unmodified 5mC residues and no 5hmC residues.

In some embodiments, the unmodified 5mC residues are oxidized to provide 5caC residues and/or 5fC residues, using any suitable means. The oxidizing agent is selected to oxidize 5mC residues beyond hydroxymethylation, i.e., to provide 5caC and/or 5fC residues. Oxidation may be carried out enzymatically, using a catalytically active TET family enzyme. A “TET family enzyme” or a “TET enzyme” as those terms are used herein refer to a catalytically active “TET family protein” or a “TET catalytically active fragment” as defined in U.S. Pat. No. 9,115,386, the disclosure of which is incorporated by reference herein. A preferred TET enzyme in this context is TET2; see Ito et al. (2011) Science 333(6047):1300-1303. Oxidation may also be carried out chemically, as described in the preceding section, using a chemical oxidizing agent. Examples of suitable oxidizing agent include, without limitation: a perruthenate anion in the form of an inorganic or organic perruthenate salt, including metal perruthenates such as potassium perruthenate (KRuO₄), tetraalkylammonium perruthenates such as tetrapropylammonium perruthenate (TPAP) and tetrabutylammonium perruthenate (TBAP), and polymer supported perruthenate (PSP); and inorganic peroxo compounds and compositions such as peroxotungstate or a copper (II) perchlorate/TEMPO combination. It is unnecessary at this point to separate 5fC-containing fragments from 5caC-containing fragments, insofar as in the next step of the process, converts both 5fC residues and 5caC residues to dihydrouracil (DHU).

In some embodiments, 5-hydroxymethylcytosine residues are blocked with β-glucosyltransferase (β3GT), while 5-methylcytosine residues are oxidized with a TET enzyme effective to provide a mixture of 5-formylcytosine and 5-carboxymethylcytosine. The mixture containing both of these oxidized species can be reacted with 2-picoline borane or another borane reducing agent to give dihydrouracil. In a variation on this embodiment, 5hmC-containing fragments are not removed. Rather, “TET-Assisted Picoline Borane Sequencing (TAPS),” 5mC-containing fragments and 5hmC-containing fragments are together enzymatically oxidized to provide 5fC- and 5caC-containing fragments. Reaction with 2-picoline borane results in DHU residues wherever 5mC and 5hmC residues were originally present. “Chemical Assisted Picoline Borane Sequencing (CAPS),” involves selective oxidation of 5hmC-containing fragments with potassium perruthenate, leaving 5mC residues unchanged.

In a related embodiment, the above method further includes identifying a hydroxymethylation pattern in the 5hmC-containing DNA removed from the cell-free DNA. This can be carried out using the techniques described in detail in WO 2017/176630. The process can be carried out without removal or isolation of intermediates in a one-tube method. For example, initially, cell-free DNA fragments, preferably adapter-ligated DNA fragments, are subjected to functionalization with βGT-catalyzed uridine diphosphoglucose 6-azide, followed by biotinylation via the chemoselective azide groups. This procedure results in covalently attached biotin at each 5hmC site. In a next step, the biotinylated strands and strands containing unmodified (native) 5mC are pulled down simultaneously for further processing. The native 5mC-containing strands are pulled down using an anti-5mC antibody or a methyl-CpG-binding domain (MBD) protein, as is known in the art. Then, with the 5hmC residues blocked, the unmodified 5mC residues are selectively oxidized using any suitable technique for converting 5mC to 5fC and/or 5caC, as described elsewhere herein.

The fragments obtained by means of the amplification can carry a directly or indirectly detectable label. In some embodiments, the labels are fluorescent labels, radionuclides, or detachable molecule fragments having a typical mass that can be detected in a mass spectrometer. Where said labels are mass labels, some embodiments provide that the labeled amplicons have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

In some embodiments, the markers described herein find use in QUARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QuARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer.

Many valuable genetic markers are present in extremely low amounts in samples and many of the events that produce such markers are rare. Consequently, even sensitive detection methods such as PCR require a large amount of DNA to provide enough of a low-abundance target to meet or supersede the detection threshold of the assay. Moreover, the presence of even low amounts of inhibitory substances can compromise the accuracy and precision of these assays directed to detecting such low amounts of a target. Accordingly, provided herein are methods providing the requisite management of volume and concentration to produce such DNA samples.

In some embodiments, the biological sample is a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample. In some embodiments, the tissue sample is an oropharyngeal tissue sample comprising one or more of soft palate cells or tissues, throat cells or tissues, tongue cells or tissues, and tonsil cells or tissues. In some embodiments, the tissue sample is an HPV(+) tissue sample. In some embodiments, the subject is a human. Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a sample (e.g., a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample) using direct gene capture, e.g., as detailed in U.S. Pat. Nos. 8,808,990 and 9,169,511, and in WO 2012/155072, or by a related method.

The analysis of markers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

Genomic DNA may be isolated by any means, including the use of commercially available kits. Briefly, wherein the DNA of interest is encapsulated by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, paraffin-embedded tissue, body fluids, stool, tissue, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Appl. Ser. No. 61/485,386 or by a related method.

The genomic DNA sample is then treated with at least one reagent, or series of reagents, which distinguishes between methylated and non-methylated CpG dinucleotides within at least one marker comprising a DMR (e.g., DMRs Tables 1, 2, 6, or 7).

In some embodiments, the reagent converts cytosine bases which are unmethylated at the 5′-position to uracil, thymine, or another base which is dissimilar to cytosine in terms of hybridization behavior. However, in some embodiments, the reagent may be a methylation sensitive restriction enzyme.

In some embodiments, the genomic DNA sample is treated in such a manner that cytosine bases that are unmethylated at the 5′ position are converted to uracil, thymine, or another base that is dissimilar to cytosine in terms of hybridization behavior. In some embodiments, this treatment is carried out with bisulfite (hydrogen sulfite, disulfite) followed by alkaline hydrolysis.

The treated nucleic acid is then analyzed to determine the methylation state of the target gene sequences (at least one gene, genomic sequence, or nucleotide from a marker comprising a DMR, e.g., at least one DMR chosen from the DMRs in Tables 1, 2, 6, or 7). The method of analysis may be selected from those known in the art, including those listed herein, e.g., QuARTS and MSP, as described herein.

Such samples can be obtained by any number of means known in the art, such as will be apparent to the skilled person. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens.

Embodiments of the present disclosure further provide compositions. In some embodiments, the present disclosure provides composition comprising a nucleic acid comprising a DMR and a bisulfite reagent. In some embodiments, composition comprising a nucleic acid comprising a DMR and one or more oligonucleotide according to SEQ ID NOS 1-176 are provided. In certain embodiments, compositions comprising a nucleic acid comprising a DMR and a methylation-sensitive restriction enzyme are provided. In certain embodiments, compositions comprising a nucleic acid comprising a DMR and a polymerase are provided.

### 3. METHODS OF TREATMENT

In some embodiments, the present disclosure provides methods for treating a subject (e.g., a patient having or suspected of having one or more types or subtypes of oropharyngeal cancer). In accordance with these embodiments, the method includes determining a methylation state or profile of one or more methylated DNA markers provided herein, and administering a treatment to the patient based on the results of determining the methylation state. The treatment may be administration of a pharmaceutical compound, a vaccine, performing a surgery, imaging the patient, performing another test. In some embodiments, treating a subject includes a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments, a method for diagnosing a specific type of cancer in a subject is provided. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example one or more biomarkers (e.g., one or more methylated markers, methylated marker genes, genes, DMRs, and/or DNA methylated markers as disclosed herein), the methylation state of which is indicative of the presence, severity, or absence of the condition.

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation state) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers (e.g., DMR) disclosed herein. Further, in some embodiments of the presently disclosed subject matter, multiple determination of the biomarkers over time can be made to facilitate diagnosis and/or prognosis. A temporal change in the biomarker can be used to predict a clinical outcome, monitor the progression of cancer or a subtype of cancer, and/or monitor the efficacy of appropriate therapies directed against the cancer. In such an embodiment, for example, one might expect to see a change in the methylation state of one or more biomarkers (e.g., DMR) disclosed herein (and potentially one or more additional biomarker(s), if monitored) in a biological sample over time during the course of an effective therapy.

The presently disclosed subject matter further provides a method for determining whether to initiate or continue prophylaxis or treatment of a cancer in a subject. In some embodiments, the method comprises providing a series of biological samples over a time period from the subject; analyzing the series of biological samples to determine a methylation state or profile of at least one marker disclosed herein in each of the biological samples; and comparing any measurable change in the methylation states of one or more of the biomarkers in each of the biological samples. Any changes over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation states can be measured in each of the samples taken from different time points and qualitative and/or quantitative differences noted. A change in the methylation states of the biomarker levels from the different samples can be correlated with a specific cancer risk, prognosis, determining treatment efficacy, and/or progression of the cancer in the subject. In some embodiments, the methods and compositions of the present disclosure are for treatment or diagnosis of disease at an early stage, for example, before symptoms of the disease appear. In some embodiments, the methods and compositions of the present disclosure are for treatment or diagnosis of disease at a clinical stage.

In some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker (e.g., a DMR and/or protein marker). Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., having a normal methylation state of one or more DMR), the chance of a given outcome (e.g., suffering from a specific type of cancer) may be very low.

In some embodiments, a statistical analysis associates a prognostic indicator with a predisposition to an adverse outcome. For example, in some embodiments, a methylation state different from that in a normal control sample obtained from a patient who does not have a cancer can signal that a subject is more likely to suffer from a cancer than subjects with a level that is more similar to the methylation state in the control sample, as determined by a level of statistical significance. Additionally, a change in methylation state from a baseline (e.g., “normal”) level can be reflective of subject prognosis, and the degree of change in methylation state can be related to the severity of adverse events. Statistical significance is often determined by comparing two or more populations and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein (e.g., a DMR; protein marker) can be established, and the degree of change in the methylation state of the biomarker in a biological sample is simply compared to the threshold degree of change in the methylation state. A preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker as a function of assay units, e.g., fluorescent signal intensity, if a fluorescent label is used. Using samples taken from multiple donors, standard curves can be provided for control methylation states of the one or more biomarkers in normal tissue, as well as for “at-risk” levels of the one or more biomarkers in plasma taken from donors with a specific type of cancer. In certain embodiments of the method, a subject is identified as having cancer upon identifying an aberrant methylation state of one or more DMRs provided herein in a biological sample obtained from the subject. In other embodiments of the method, the detection of an aberrant methylation state of one or more of such biomarkers in a biological sample obtained from the subject results in the subject being identified as having cancer.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having a specific type of cancer if, when compared to a control methylation state, there is a measurable difference in the methylation state of at least one biomarker in the sample. Conversely, when no change in methylation state is identified in the biological sample, the subject can be identified as not having a specific type of cancer, not being at risk for the cancer, or as having a low risk of the cancer. In this regard, subjects having the cancer or risk thereof can be differentiated from subjects having low to substantially no cancer or risk thereof. Those subjects having a risk of developing a specific type of cancer can be placed on a more intensive and/or regular screening schedule. On the other hand, those subjects having low to substantially no risk may avoid being subjected to additional testing for cancer risk (e.g., invasive procedure), until such time as a future screening, for example, a screening conducted in accordance with the various embodiments of the present disclosure, indicates that a risk of cancer risk has appeared in those subjects.

As mentioned above, depending on the embodiment of the method of the present disclosure, detecting a change in methylation state of the one or more biomarkers can be a qualitative determination or it can be a quantitative determination. As such, the step of diagnosing a subject as having, or at risk of developing, a specific type of cancer indicates that certain threshold measurements are made, e.g., the methylation state of the one or more biomarkers in the biological sample varies from a predetermined control methylation state. In some embodiments of the method, the control methylation state is any detectable methylation state of the biomarker. In other embodiments of the method where a control sample is tested concurrently with the biological sample, the predetermined methylation state is the methylation state in the control sample. In other embodiments of the method, the predetermined methylation state is based upon and/or identified by a standard curve. In other embodiments of the method, the predetermined methylation state is a specifically state or range of state. As such, the predetermined methylation state can be chosen, within acceptable limits that will be apparent to those skilled in the art, based in part on the embodiment of the method being practiced and the desired specificity, etc.

Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, embodiments of the present disclosure provide for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; and horses. Thus, also provided is the diagnosis and treatment of livestock, including, but not limited to, domesticated swine, ruminants, ungulates, horses (including racehorses), and the like.

### 4. SAMPLES, KITS, AND CONTROLS

Embodiments of the present disclosure provide technology for screening one or more types of oropharyngeal cancer from a biological sample. In accordance with these embodiments, the present disclosure includes, but is not limited to, methods and compositions for detecting the presence of one or more types and/or subtypes of oropharyngeal cancer from a biological sample. In some embodiments, the biological sample is a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample. In some embodiments, the tissue sample is an oropharyngeal tissue sample comprising one or more of soft palate cells or tissues, throat cells or tissues, tongue cells or tissues, and tonsil cells or tissues. In some embodiments, the tissue sample is an HPV(+) tissue sample. In some embodiments, the subject is a human.

In other embodiments, “sample,” “test sample,” and “biological sample” refer to fluid sample containing or suspected of containing a methylated DNA marker of the present disclosure. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis. In a particular example, the source is a mammalian (e.g., human) bodily substance (e.g., bodily fluid, blood such as whole blood, serum, plasma, urine, saliva, sweat, sputum, semen, mucus, lacrimal fluid, lymph fluid, amniotic fluid, interstitial fluid, cerebrospinal fluid, feces, tissue, organ, one or more dried blood spots, or the like). Tissues may include, but are not limited to, an oropharyngeal tissue sample comprising one or more of soft palate cells or tissues, throat cells or tissues, tongue cells or tissues, and tonsil cells or tissues. The sample may be a liquid sample or a liquid extract of a solid sample. In some embodiments, the source of the sample may be an organ or tissue, such as a biopsy sample and/or a secretion sample (e.g., oropharyngeal secretion), which may be solubilized by tissue disintegration/cell lysis. Additionally, the sample can be a nasopharyngeal or oropharyngeal sample obtained using one or more swabs that, once obtained, is placed in a sterile tube containing a virus transport media (VTM) or universal transport media (UTM), for testing.

A wide range of volumes of the fluid sample may be analyzed. In a few exemplary embodiments, the sample volume may be about 0.5 nL, about 1 nL, about 3 nL, about 0.01 L, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL.

In some cases, the fluid sample may be diluted prior to use in an assay. For example, in embodiments where the source containing a methylated DNA marker is a human body fluid (e.g., blood, serum, secretion), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments where the source containing a methylated DNA marker is a human body fluid (e.g., blood, serum, secretion), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

It may be desirable to include a control. The control may be analyzed concurrently with the sample from the subject as described above. The results obtained from the subject sample can be compared to the results obtained from the control sample. Standard curves may be provided, with which assay results for the sample may be compared. Such standard curves present levels of one or more methylated DNA markers as a function of assay units. Using samples taken from multiple donors, standard curves can be provided for reference levels of a methylated DNA marker in normal healthy tissue, as well as for “at-risk” levels of the methylated DNA marker in tissue taken from donors, who may have one or more characteristics of an oropharyngeal cancer.

Embodiments of the present disclosure also include a kit for performing the methods described herein. The kits comprise embodiments of the compositions, devices, apparatuses, etc. described herein, and instructions for use of the kit. Such instructions describe appropriate methods for preparing an analyte from a sample, e.g., for collecting a sample and preparing a nucleic acid from the sample. Individual components of the kit are packaged in appropriate containers and packaging (e.g., vials, boxes, blister packs, ampules, jars, bottles, tubes, and the like) and the components are packaged together in an appropriate container (e.g., a box or boxes) for convenient storage, shipping, and/or use by the user of the kit. It is understood that liquid components (e.g., a buffer) may be provided in a lyophilized form to be reconstituted by the user. Kits may include a control or reference for assessing, validating, and/or assuring the performance of the kit. For example, a kit for assaying the amount of a nucleic acid present in a sample may include a control comprising a known concentration of the same or another nucleic acid for comparison and, in some embodiments, a detection reagent (e.g., a primer) specific for the control nucleic acid. The kits are appropriate for use in a clinical setting and, in some embodiments, for use in a user's home. The components of a kit, in some embodiments, provide the functionalities of a system for preparing a nucleic acid solution from a sample. In some embodiments, certain components of the system are provided by the user.

In some embodiments, the present disclosure provides compositions (e.g., reaction mixtures). In some embodiments, the present disclosure provides a composition comprising a nucleic acid comprising a DMR and a reagent capable of modifying DNA in a methylation-specific manner (e.g., a methylation-sensitive restriction enzyme, a methylation-dependent restriction enzyme, and a bisulfite reagent) (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), borane reducing agent). Some embodiments provide a composition comprising a nucleic acid comprising a DMR and an oligonucleotide as described herein. Some embodiments provide a composition comprising a nucleic acid comprising a DMR and a methylation-sensitive restriction enzyme. Some embodiments provide a composition comprising a nucleic acid comprising a DMR and a polymerase.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for determining a methylation state (e.g., of one or more DMRs in Tables 1, 2, 6, or 7); comparing methylation states; generating standard curves; determining a Ct value; calculating a fraction, frequency, or percentage of methylation; identifying a CpG island; determining a specificity and/or sensitivity of an assay or marker; calculating an ROC curve and an associated AUC; sequence analysis; all as described herein or is known in the art. In some embodiments, a microprocessor is part of a system for determining a methylation state (e.g., of one or more DMRs in Tables 1, 2, 6, or 7); comparing methylation states; generating standard curves; determining a Ct value; calculating a fraction, frequency, or percentage of methylation; identifying a CpG island; determining a specificity and/or sensitivity of an assay or marker; calculating an ROC curve and an associated AUC; sequence analysis; all as described herein or is known in the art.

In some embodiments, a software or hardware component receives the results of multiple assays and determines a single value result to report to a user that indicates a cancer risk based on the results of the multiple assays (e.g., determining the methylation state of one or more DMRs in Tables 1, 2, 6, or 7). Related embodiments calculate a risk factor based on a mathematical combination (e.g., a weighted combination, a linear combination) of the results from the multiple assays (e.g., determining the methylation state of one or more DMRs in Tables 1, 2, 6, or 7). In some embodiments, the methylation state of a DMR defines a dimension and may have values in a multidimensional space and the coordinate defined by the methylation states of multiple DMRs is a result (e.g., to report to a user, or related to a cancer risk).

In some embodiments, the various embodiments of the present disclosure are associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, California and Motorola Corporation of Schaumburg, Illinois. Such processors include, or may be in communication with, media, for example computer-readable media, which stores instructions that, when executed by the processor, cause the processor to perform the steps described herein.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device. All such components, computers, and systems described herein as associated with the technology may be logical or virtual.

In some embodiments, the present disclosure provides systems for screening for one or more types or subtypes of oropharyngeal cancer in a sample obtained from a subject. Exemplary embodiments of systems include, e.g., a system for screening for multiple types or subtypes of oropharyngeal cancer in a sample obtained from a subject (e.g., a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample). In some embodiments, the system comprises an analysis component configured to one or both of determining the methylation state of one or more methylated markers in a sample, a software component configured to compare the methylation state of the one or more methylated markers in the sample with a control sample or a reference sample recorded in a database, and an alert component configured to alert a user of a cancer associated state.

In some embodiments, an alert is determined by a software component that receives the results from multiple assays (e.g., determining the methylation states of the one or more methylated markers) and calculating a value or result to report based on the multiple results.

Some embodiments provide a database of weighted parameters associated with each methylated marker provided herein for use in calculating a value or result and/or an alert to report to a user (e.g., such as a physician, nurse, clinician, etc.). In some embodiments all results from multiple assays are reported. In some embodiments, one or more results are used to provide a score, value, or result based on a composite of one or more results from multiple assays that is indicative of a cancer risk in a subject. Such methods are not limited to particular methylation markers. In such methods and systems, the one or more methylation markers comprise a base in a DMR selected from the DMRs in Tables 1, 2, 6, and 7.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

The various components of the kit optionally are provided in suitable containers as necessary. The kit can further include containers for holding or storing a sample (e.g., a container or cartridge for a urine, whole blood, plasma, serum sample, tissue, or bodily secretion sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like. In some embodiments, the instrument is a collection device. In some embodiments, the biological sample is obtained from the subject, and the method further comprises extracting the DNA sample from the biological sample using an extraction element. In some embodiments, the biological sample is collected with a collection device having an absorbing element capable of collecting the biological sample upon contact. In some embodiments, the absorbing element is a sponge configured for insertion into an orifice (e.g., mouth, throat, or nose).

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Experiments were conducted to assess the feasibility of a panel of differentiated methylated regions (DMRs) for detecting oropharyngeal cancer (e.g., HPV⁺ oropharyngeal squamous cell cancer). These regions are listed below in Table 1.

TABLE 1
Methylated regions for identifying oropharyngeal cancer (e.g., HPV⁺
oropharyngeal squamous cell cancer) (genomic coordinates for the regions
shown are based on the Human February 2009 (GRCh37/hg19) Assembly).

| DMRs | | Chromosome | Chromosome | |
|---|---|---|---|---|
| (RCC DMR) | Gene Annotation | No. | Coordinates | Gene Title |
| 1 | ABCB1 | 7 | 87257074- | ATP-dependent translocase |
| | | | 87257239 | ABCB1 |
| 2 | ARHGAP12 | 10 | 32218791- | Rho GTPase-activating |
| | | | 32218902 | protein 12 |
| 3 | ASCL1 | 12 | 103351856- | Achaete-scute homolog 1 |
| | | | 103352059 | |
| 4 | C1orf114_A | 1 | 169396696- | Chromosome 1 Open Reading |
| | (C1orf114_6696) | | 169396795 | Frame 141 |
| 5 | EMX1 | 2 | 73147710- | Homeobox protein EMX1 |
| | | | 73147772 | |
| 6 | GRIN2D | 19 | 48918144- | Glutamate Ionotropic Receptor |
| | | | 48918350 | NMDA Type Subunit 2D |
| 7 | LOC645323 (region 3; | 5 | 87970706- | — |
| | LOC645323_0706) | | 87970894 | |
| 8 | MAX.chr6.58147682- | 6 | 58147682- | — |
| | 58147771 | | 58147771 | |
| 9 | MAX.chr9.36739811- | 9 | 36739811- | — |
| | 36739868 | | 36739868 | |
| 10 | NEUROG3 | 10 | 71332209- | Neurogenin-3 |
| | | | 71332333 | |
| 11 | NID2 | 14 | 52535699- | Nidogen-2 |
| | | | 52536126 | |
| 12 | TBX15 | 1 | 119527066- | T-box transcription factor |
| | | | 119527655 | TBX15 |
| 13 | TMEM200C | 18 | 5890757- | Transmembrane protein 200C |
| | | | 5890849 | |
| 14 | TSPYL5 | 8 | 98289858- | Testis-specific Y-encoded-like |
| | | | 98290220 | protein 5 |
| 15 | TTYH1 | 19 | 54926431- | Protein tweety homolog 1 |
| | | | 54926953 | |
| 16 | VWC2 | 7 | 49813135- | Von Willebrand Factor C Domain |
| | | | 49814168 | Containing 2 |
| 17 | ZNF610 | 19 | 52839503- | Zinc finger protein 610 |
| | | | 52840013 | |
| 18 | ZNF69 | 19 | 11998671- | Zinc finger protein 69 |
| | | | 11998972 | |
| 19 | ZNF773 | 19 | 58011241- | Zinc finger protein 773 |
| | | | 58011636 | |
| 20 | ZNF781_A | 19 | 38182950- | Zinc finger protein 781 |
| | (ZNF781_2950A) | | 38183127 | |
| 21 | ZNF781_B | 19 | 38182950- | Zinc finger protein 781 |
| | (ZNF781_2950B) | | 38183230 | |
| 22 | ALX4 | 11 | 44326332- | ALX homeobox 4 |
| | | | 44326444 | |
| 23 | ATP10A | 15 | 26108236- | ATPase, class V, type 10A |
| | | | 26108573 | |
| 24 | C1orf114_B | 1 | 169396622- | chromosome 1 open reading |
| | (C1orf114_6622) | | 169396765 | frame 114 |
| 25 | C1QL3 | 10 | 16562329- | complement component 1, q |
| | | | 16562405 | subcomponent-like 3 |
| 26 | CA8 | 8 | 61193337- | carbonic anhydrase VIII |
| | | | 61193995 | |
| 27 | CACNA1A | 19 | 13617008- | calcium channel, voltage- |
| | | | 13617106 | dependent, P/Q type, alpha 1A |
| | | | | subunit |
| 28 | CACNG8 | 19 | 54485837- | calcium channel, voltage- |
| | | | 54485846 | dependent, gamma subunit 8 |
| 29 | CALCA | 11 | 14995266- | calcitonin-related polypeptide |
| | | | 14995572 | alpha |
| 30 | CCNA1 | 13 | 37006107- | cyclin A1 |
| | | | 37006678 | |
| 31 | CLIC6 | 21 | 36041893- | chloride intracellular |
| | | | 36041956 | channel 6 |
| 32 | CLSTN2 | 3 | 139653792- | calsyntenin 2 |
| | | | 139654257 | |
| 33 | CR1 | 1 | 207669489- | complement component (3b/4b) |
| | | | 207669878 | receptor 1 (Knops blood group) |
| 34 | CTNND2 | 5 | 11904006- | catenin (cadherin-associated |
| | | | 11904366 | protein), delta 2 (neural |
| | | | | plakophilin-related arm-repeat |
| | | | | protein) |
| 35 | DAB1 | 1 | 58715813- | disabled homolog 1 (Drosophila) |
| | | | 58716179 | |
| 36 | DGKG | 3 | 186079645- | diacylglycerol kinase, gamma |
| | | | 186079768 | 90 kDa |
| 37 | DOK1 | 2 | 74782446- | docking protein 1, 62 kDa |
| | | | 74782597 | (downstream of tyrosine kinase 1) |
| 38 | DOK6 | 18 | 67068039- | docking protein 6 |
| | | | 67068223 | |
| 39 | DPP4 | 2 | 162930234- | dipeptidyl-peptidase 4 |
| | | | 162930577 | |
| 40 | DUXA | 19 | 57683241- | double homeobox A |
| | | | 57683288 | |
| 41 | ELMO1 (region 1; | 7 | 37487417- | engulfment and cell motility 1 |
| | ELMO1_7417) | | 37488625 | |
| 42 | EMBP1 | 1 | 121260881- | embigin pseudogene 1 |
| | | | 121261189 | |
| 43 | EPDR1 (region 1; | 7 | 37960389- | ependymin related protein 1 |
| | EPDR1_0389) | | 37960975 | (zebrafish) |
| 44 | FGF12 | 3 | 192125977- | fibroblast growth factor 12 |
| | | | 192126052 | |
| 45 | FLJ43390 | 14 | 62583913- | hypothetical LOC646113 |
| | | | 62584109 | |
| 46 | FMN2 | 1 | 240255182- | formin 2 |
| | | | 240255338 | |
| 47 | FOXB2 | 9 | 79633392- | forkhead box B2 |
| | | | 79633419 | |
| 48 | FOXD4 | 9 | 117499- | forkhead box D4 |
| | | | 117614 | |
| 49 | FREM3 | 4 | 144621581- | FRAS1 related extracellular |
| | | | 144621670 | matrix 3 |
| 50 | GALR1 | 18 | 74962243- | galanin receptor 1 |
| | | | 74962454 | |
| 51 | GDF6 | 8 | 97171935- | growth differentiation factor 6 |
| | | | 97172016 | |
| 52 | GFRA1 | 10 | 118031547- | GDNF family receptor alpha 1 |
| | | | 118031694 | |
| 53 | GRIK3 | 1 | 37498816- | glutamate receptor, ionotropic, |
| | | | 37498828 | kainate 3 |
| 54 | HOXB3 | 17 | 46655387- | homeobox B3 |
| | | | 46656122 | |
| 55 | HOXB4 | 17 | 46659595- | homeobox B4 |
| | | | 46659703 | |
| 56 | HPSE2 | 10 | 100993583- | heparanase 2 |
| | | | 100993598 | |
| 57 | LDLRAD2 | 1 | 22140984- | low density lipoprotein receptor |
| | | | 22141213 | class A domain containing 2 |
| 58 | LHX2 | 9 | 126780041- | LIM homeobox 2 |
| | | | 126780896 | |
| 59 | LOC100131366 | 14 | 103655316- | hypothetical LOC100131366 |
| | | | 103655503 | |
| 60 | LOC345643 | 5 | 54516736- | hypothetical LOC345643 |
| | | | 54516809 | |
| 61 | LOC386758 | 19 | 56904686- | — |
| | | | 56905053 | |
| 62 | LOC645323 (region 1; | 5 | 87956761- | hypothetical LOC645323 |
| | LOC645323_6761) | | 87957004 | |
| 63 | LOC645323 (region 2; | 5 | 87971583- | hypothetical LOC645323 |
| | LOC645323_1583) | | 87971822 | |
| 64 | LOC648809 | 15 | 84748956- | elongation factor Tu GTP-binding |
| | | | 84749041 | domain-containing protein 1 |
| | | | | pseudogene |
| 65 | LOC728392 | 17 | 5404375- | hypothetical protein LOC728392 |
| | | | 5404508 | |
| 66 | MAML3 | 4 | 140656525- | mastermind-like 3 (Drosophila) |
| | | | 140656723 | |
| 67 | MAPRE2 | 18 | 32557478- | microtubule-associated protein, |
| | | | 32557725 | RP/EB family, member 2 |
| 68 | MAX.chr1.226288154- | 1 | 226288154- | — |
| | 226288189 | | 226288189 | |
| 69 | MAX.chr1.2375078- | 1 | 2375078- | — |
| | 2375126 | | 2375126 | |
| 70 | MAX.chr1.241587339- | 1 | 241587339- | — |
| | 241587784 | | 241587784 | |
| 71 | MAX.chr1.50798781- | 1 | 50798781- | — |
| | 50799423 | | 50799423 | |
| 72 | MAX.chr10.22765150- | 10 | 22765150- | — |
| | 22765477 | | 22765477 | |
| 73 | MAX.chr10.23462342- | 10 | 23462342 | — |
| | 23462436 | | 23462436 | |
| 74 | MAX.chr11.14926602- | 11 | 14926602- | — |
| | 14927044 | | 14927044 | |
| 75 | MAX.chr11.58903531- | 11 | 58903531- | — |
| | 58903592 | | 58903592 | |
| 76 | MAX.chr13.28527984- | 13 | 28527984- | — |
| | 28528214 | | 28528214 | |
| 77 | MAX.chr13.29106641- | 13 | 29106641- | — |
| | 29107037 | | 29107037 | |
| 78 | MAX.chr14.100784488- | 14 | 100784488- | — |
| | 100784782 | | 100784782 | |
| 79 | MAX.chr16.3221176- | 16 | 3221176- | — |
| | 3221223 | | 3322122 | |
| 80 | MAX.chr16.3222040- | 16 | 3222040- | — |
| | 3222098 | | 3222098 | |
| 81 | MAX.chr16.71460171- | 16 | 71460171- | — |
| | 71460282 | | 71460282 | |
| 82 | MAX.chr19.11805263- | 19 | 11805263- | — |
| | 11805639 | | 11805639 | |
| 83 | MAX.chr19.16394457- | 19 | 16394457- | — |
| | 16394646 | | 16394646 | |
| 84 | MAX.chr19.21657626- | 19 | 21657626- | — |
| | 21657769 | | 21657769 | |
| 85 | MAX.chr19.22034646- | 19 | 22034646- | — |
| | 22034887 | | 22034887 | |
| 86 | MAX.chr19.23299989- | 19 | 23299989- | — |
| | 23300156 | | 23300156 | |
| 87 | MAX.chr19.30713427- | 19 | 30713427- | — |
| | 30713588 | | 30713588 | |
| 88 | MAX.chr19.30716926- | 19 | 30716926- | — |
| | 30717074 | | 30717074 | |
| 89 | MAX.chr19.30718373- | 19 | 30718373- | — |
| | 30719719 | | 30719719 | |
| 90 | MAX.chr2.118981724- | 2 | 118981724- | — |
| | 118982174 | | 118982174 | |
| 91 | MAX.chr2.127783107- | 2 | 127783107- | — |
| | 127783403 | | 127783403 | |
| 92 | MAX.chr2.173099712- | 2 | 173099712- | — |
| | 173099791 | | 173099791 | |
| 93 | MAX.chr2.66808635- | 2 | 66808635- | — |
| | 66808731 | | 66808731 | |
| 94 | MAX.chr22.50064113- | 22 | 50064113- | — |
| | 50064259 | | 50064259 | |
| 95 | MAX.chr3.137489884- | 3 | 137489884- | — |
| | 137490061 | | 137490061 | |
| 96 | MAX.chr5.138923141- | 5 | 138923141- | — |
| | 138923219 | | 138923219 | |
| 97 | MAX.chr5.42995180- | 5 | 42995180- | — |
| | 42995535 | | 42995535 | |
| 98 | MAX.chr6.38683091- | 6 | 38683091- | — |
| | 38683226 | | 38683226 | |
| 99 | MAX.chr7.121952014- | 7 | 121952014- | — |
| | 121952084 | | 121952084 | |
| 100 | MAX.chr7.155166980- | 7 | 155166980- | — |
| | 155167310 | | 155167310 | |
| 101 | MAX.chr8.99986792- | 8 | 99986792- | — |
| | 99986864 | | 99986864 | |
| 102 | MAX.chr9.79627078- | 9 | 79627078- | — |
| | 79627116 | | 79627116 | |
| 103 | MAX.chr9.79638034- | 9 | 79638034- | — |
| | 79638077 | | 79638077 | |
| 104 | MAX.chr9.98789824- | 9 | 98789824- | — |
| | 98789847 | | 98789847 | |
| 105 | MDFI | 6 | 41606290- | MyoD family inhibitor |
| | | | 41606487 | |
| 106 | MECOM | 3 | 169376578- | MDS1 and EVIL complex locus |
| | | | 169376702 | |
| 107 | MED12L | 3 | 150804663- | mediator complex subunit 12-like |
| | | | 150805021 | |
| 108 | MIR129-2 | 11 | 43602934- | microRNA 129-2 |
| | | | 43603032 | |
| 109 | MIR196A1 | 17 | 46711335- | microRNA 196a-1 |
| | | | 46711359 | |
| 110 | NELL1 | 11 | 20691446- | NEL-like 1 (chicken) |
| | | | 20691520 | |
| 111 | NPY | 7 | 24323248- | neuropeptide Y |
| | | | 24323319 | |
| 112 | ONECUT2 | 18 | 55105693- | one cut homeobox 2 |
| | | | 55105794 | |
| 113 | OPCML | 11 | 132952679- | opioid binding protein/cell |
| | | | 132952966 | adhesion molecule-like |
| 114 | PARP15 | 3 | 122296579- | poly (ADP-ribose) polymerase |
| | | | 122296700 | family, member 15 |
| 115 | PDGFD | 11 | 104034648- | platelet derived growth factor D |
| | | | 104035133 | |
| 116 | PEX5L | 3 | 179754874- | peroxisomal biogenesis factor 5- |
| | | | 179754929 | like |
| 117 | PRR15 | 7 | 29605829- | proline rich 15 |
| | | | 29606153 | |
| 118 | SEMA6A | 5 | 115912116- | sema domain, transmembrane |
| | | | 115912302 | domain (TM), and cytoplasmic |
| | | | | domain, (semaphorin) 6A |
| 119 | SFMBT2 | 10 | 7450191- | Scm-like with four mbt domains |
| | | | 7450327 | 2 |
| 120 | SGIP1 | 1 | 66998873- | SH3-domain GRB2-like |
| | | | 66999034 | (endophilin) interacting protein 1 |
| 121 | SIM2 | 21 | 38120049- | single-minded homolog 2 |
| | | | 38120250 | (Drosophila) |
| 122 | SLC35F3 | 1 | 234040507- | solute carrier family 35, member |
| | | | 234040529 | F3 |
| 123 | SLCO4C1 | 5 | 101632236- | solute carrier organic anion |
| | | | 101632315 | transporter family, member 4C1 |
| 124 | SORCS3 (region 1; | 10 | 106400952- | sortilin-related VPS10 domain |
| | SORCS3_0952) | | 106401370 | containing receptor 3 |
| 125 | SORCS3 (region 2; | 10 | 106402558- | sortilin-related VPS10 domain |
| | SORCS3_2558) | | 106402652 | containing receptor 3 |
| 126 | ST6GALNAC5 | 1 | 77333925- | ST6 (alpha-N-acetyl-neuraminyl- |
| | | | 77334022 | 2,3-beta-galactosyl-1,3)-N- |
| | | | | acetylgalactosaminide alpha-2,6- |
| | | | | sialyltransferase 5 |
| 127 | ST8SIA5 | 18 | 44336443- | ST8 alpha-N-acetyl-neuraminide |
| | | | 44337094 | alpha-2,8-sialyltransferase 5 |
| 128 | SV2C | 5 | 75379004- | synaptic vesicle glycoprotein 2C |
| | | | 75379253 | |
| 129 | TACC2 | 10 | 123923132- | transforming, acidic coiled-coil |
| | | | 123923269 | containing protein 2 |
| 130 | TFAP2E | 1 | 36043354- | transcription factor AP-2 epsilon |
| | | | 36043427 | (activating enhancer binding |
| | | | | protein 2 epsilon) |
| 131 | TLX2 | 2 | 74741063- | T-cell leukemia homeobox 2 |
| | | | 74741295 | |
| 132 | TLX3 | 5 | 170738892- | T-cell leukemia homeobox 3 |
| | | | 170739145 | |
| 133 | TRH | 3 | 129693355- | thyrotropin-releasing hormone |
| | | | 129693854 | |
| 134 | TRIM58 | 1 | 248020543- | tripartite motif-containing 58 |
| | | | 248021004 | |
| 135 | VAV3 | 1 | 108508521- | vav 3 guanine nucleotide |
| | | | 108508601 | exchange factor |
| 136 | VSTM2B (region 1; | 19 | 30016096- | V-set and transmembrane domain |
| | VSTM2B_6096) | | 30016161 | containing 2B |
| 137 | VSTM2B (region 2; | 19 | 30017171- | V-set and transmembrane domain |
| | VSTM2B_7171) | | 30018338 | containing 2B |
| 138 | WDR17 | 4 | 176987103- | WD repeat domain 17 |
| | | | 176987241 | |
| 139 | ZNF254 | 19 | 24269958- | zinc finger protein 254 |
| | | | 24270038 | |
| 140 | ZNF43 | 19 | 22018638- | zinc finger protein 43 |
| | | | 22019004 | |
| 141 | ZNF486 | 19 | 20278275- | zinc finger protein 486 |
| | | | 20278343 | |
| 142 | ZNF491 | 19 | 11909233- | zinc finger protein 491 |
| | | | 11909959 | |
| 143 | ZNF518B | 4 | 10463044- | zinc finger protein 518B |
| | | | 10463098 | |
| 144 | ZNF542 | 19 | 56879626- | zinc finger protein 542 |
| | | | 56879699 | |
| 145 | ZNF625 | 19 | 12267145- | zinc finger protein 625 |
| | | | 12267309 | |
| 146 | ZNF665 (region 1; | 19 | 53696497- | zinc finger protein 665 |
| | ZNF665_6497) | | 53696704 | |
| 147 | ZNF665 (region 2; | 19 | 53700173- | zinc finger protein 665 |
| | ZNF665_0173) | | 53700459 | |
| 148 | ZNF671 | 19 | 58238740- | zinc finger protein 671 |
| | | | 58239004 | |
| 149 | ZNF763 | 19 | 12075727- | zinc finger protein 763 |
| | | | 12076257 | |
| 150 | ZNF844 | 19 | 12175505- | zinc finger protein 844 |
| | | | 12176056 | |
| 151 | AGRN | 1 | 969518- | agrin |
| | | | 969570 | |
| 152 | ANKRD35 | 1 | 145562791- | ankyrin repeat domain 35 |
| | | | 145562917 | |
| 153 | ARHGAP27 | 17 | 43487568- | Rho GTPase activating protein 27 |
| | | | 43487675 | |
| 154 | ARHGAP30 | 1 | 161039379- | Rho GTPase activating protein 30 |
| | | | 161039594 | |
| 155 | BCL2L11 | 2 | 111876417- | BCL2-like 11 (apoptosis |
| | | | 111877177 | facilitator) |
| 156 | BIN2 | 12 | 51717948- | bridging integrator 2 |
| | | | 51718130 | |
| 157 | C10orf114 | 10 | 21789269- | chromosome 10 open reading |
| | | | 21789327 | frame 114 |
| 158 | C4orf31 | 4 | 121992389- | chromosome 4 open reading |
| | | | 121992492 | frame 31 |
| 159 | C6orf132 | 6 | 42072052- | chromosome 6 open reading |
| | | | 42072400 | frame 132 |
| 160 | C6orf186 | 6 | 110679348- | chromosome 6 open reading |
| | | | 110679376 | frame 186 |
| 161 | CCDC88B | 11 | 64108181- | coiled-coil domain containing |
| | | | 64108246 | 88B |
| 162 | CRHBP | 5 | 76249579- | corticotropin releasing hormone |
| | | | 76249699 | binding protein |
| 163 | DAPK1 | 9 | 90113698- | death-associated protein |
| | | | 90113817 | kinase 1 |
| 164 | DNMT3A | 2 | 25499945- | DNA (cytosine-5-)- |
| | | | 25500298 | methyltransferase 3 alpha |
| 165 | DPP10 | 2 | 115919021- | dipeptidyl-peptidase 10 (non- |
| | | | 115919041 | functional) |
| 166 | ELMO1 (region 2; | 7 | 37392953- | engulfment and cell motility 1 |
| | ELMO1_2953) | | 37393050 | |
| 167 | EPDR1 (region 2; | 7 | 37956117- | ependymin related protein 1 |
| | EPDR1_6117) | | 37956303 | (zebrafish) |
| 168 | FAM19A2 | 12 | 62584870- | family with sequence similarity |
| | | | 62585101 | 19 (chemokine (C-C motif)-like), |
| | | | | member A2 |
| 169 | FLJ45983 | 10 | 8097589- | hypothetical LOC399717 |
| | | | 8098039 | |
| 170 | FOSL1 | 11 | 65661516- | FOS-like antigen 1 |
| | | | 65661709 | |
| 171 | FOXB1 | 15 | 60296761- | forkhead box B1 |
| | | | 60296789 | |
| 172 | GREM1 | 15 | 33011110- | gremlin 1 |
| | | | 33011126 | |
| 173 | HMHA1 | 19 | 1068372- | histocompatibility (minor) |
| | | | 1068513 | HA-1 |
| 174 | HOXA9 | 7 | 27205107- | homeobox A9 |
| | | | 27205214 | |
| 175 | IFFO1 | 12 | 6665226- | intermediate filament family |
| | | | 6665425 | orphan 1 |
| 176 | INPP4B | 4 | 143767040- | inositol polyphosphate-4- |
| | | | 143767086 | phosphatase, type II, 105 kDa |
| 177 | ITGB2 | 21 | 46340400- | integrin, beta 2 (complement |
| | | | 46340636 | component 3 receptor 3 and 4 |
| | | | | subunit) |
| 178 | ITGB4 | 17 | 73749619- | integrin, beta 4 |
| | | | 73750129 | |
| 179 | ITPKB | 1 | 226862915- | inositol 1,4,5-trisphosphate 3- |
| | | | 226863021 | kinase B |
| 180 | KCNIP2 | 10 | 103603570- | Kv channel interacting protein 2 |
| | | | 103603685 | |
| 181 | KLHDC7B | 22 | 50986987- | kelch domain containing 7B |
| | | | 50987042 | |
| 182 | LAT | 16 | 28996171- | linker for activation of T cells |
| | | | 28996363 | |
| 183 | LHX6 | 9 | 124981702- | LIM homeobox 6 |
| | | | 124981939 | |
| 184 | LIMK1 | 7 | 73508970- | LIM domain kinase 1 |
| | | | 73509149 | |
| 185 | LOC100128239 | 11 | 133906819- | hypothetical LOC100128239 |
| | | | 133906985 | |
| 186 | LOC100192379 | 4 | 122686355- | hypothetical LOC100192379 |
| | | | 122686433 | |
| 187 | LOC646278 | 15 | 29077633- | hypothetical LOC646278 |
| | | | 29077650 | |
| 188 | MAP2K2 | 19 | 4108818- | mitogen-activated protein |
| | | | 4108937 | kinase 2 |
| 189 | MAX.chr1.210426156- | 1 | 210426156- | — |
| | 210426257 | | 210426257 | |
| 190 | MAX.chr1.84326495- | 1 | 84326495- | — |
| | 84326656 | | 84326656 | |
| 191 | MAX.chr10.119312785- | 10 | 119312785- | — |
| | 119312882 | | 119312882 | |
| 192 | MAX.chr15.67326025- | 15 | 67326025- | — |
| | 67326060 | | 67326060 | |
| 193 | MAX.chr16.54316401- | 16 | 54316401- | — |
| | 54316453 | | 54316453 | |
| 194 | MAX.chr16.85482306- | 16 | 85482306- | — |
| | 85482494 | | 85482494 | |
| 195 | MAX.chr17.74994454- | 17 | 74994454- | — |
| | 74994572 | | 74994572 | |
| 196 | MAX.chr17.76339840- | 17 | 76339840- | — |
| | 76339972 | | 76339972 | |
| 197 | MAX.chr2.7571082- | 2 | 7571082- | — |
| | 7571136 | | 7571136 | |
| 198 | MAX.chr21.45577347- | 21 | 45577347- | — |
| | 45577679 | | 45577679 | |
| 199 | MAX.chr3.14852538- | 3 | 14852538- | — |
| | 14852568 | | 14852568 | |
| 200 | MAX.chr3.187676564- | 3 | 187676564- | — |
| | 187676668 | | 187676668 | |
| 201 | MAX.chr4.174430662- | 4 | 174430662- | — |
| | 174430793 | | 174430793 | |
| 202 | MAX.chr5.177411809- | 5 | 177411809- | — |
| | 177411836 | | 177411836 | |
| 203 | MAX.chr6.45631561- | 6 | 45631561- | — |
| | 45631625 | | 45631625 | |
| 204 | MAX.chr7.25892382- | 7 | 25892382- | — |
| | 25892451 | | 25892451 | |
| 205 | MAX.chr7.402563- | 7 | 402563- | — |
| | 402641 | | 402641 | |
| 206 | MAX.chr7.64349554- | 7 | 64349554- | — |
| | 64349606 | | 64349606 | |
| 207 | MAX.chr8.142046239- | 8 | 142046239- | — |
| | 142046398 | | 142046398 | |
| 208 | MAX.chr8.145900842- | 8 | 145900842- | — |
| | 145901246 | | 145901246 | |
| 209 | MAX.chr9.126101804- | 9 | 126101804- | — |
| | 126101848 | | 126101848 | |
| 210 | MAX.chr9.126978999- | 9 | 126978999- | — |
| | 126979182 | | 126979182 | |
| 211 | MAX.chr9.36458633- | 9 | 36458633- | — |
| | 36458725 | | 36458725 | |
| 212 | MAX.chr9.87905315- | 9 | 87905315- | — |
| | 87905326 | | 87905326 | |
| 213 | MBP | 18 | 74818442- | myelin basic protein |
| | | | 74818536 | |
| 214 | MFNG | 22 | 37881211- | MFNG O-fucosylpeptide 3-beta- |
| | | | 37881422 | N-acetylglucosaminyltransferase |
| 215 | MT1A (region 1; | 16 | 56669553- | metallothionein 1A |
| | MT1A_9553) | | 56669589 | |
| 216 | MT1A (region 2; | 16 | 56672245- | metallothionein 1A |
| | MT1A_2245) | | 56672278 | |
| 217 | MT1IP | 16 | 56709938- | metallothionein 1I (pseudogene) |
| | | | 56709985 | |
| 218 | NCOR2 (region 1; | 12 | 124941781- | nuclear receptor corepressor 2 |
| | NCOR2_1781) | | 124941955 | |
| 219 | NCOR2 (region 2; | 12 | 124950925- | nuclear receptor corepressor 2 |
| | NCOR2_0925) | | 124950997 | |
| 220 | NFATC1 | 18 | 77159477- | nuclear factor of activated T- |
| | | | 77159778 | cells, cytoplasmic, calcineurin- |
| | | | | dependent 1 |
| 221 | NKX3-2 | 4 | 13543800- | NK3 homeobox 2 |
| | | | 13543846 | |
| 222 | NRN1 | 6 | 6004182- | neuritin 1 |
| | | | 6004237 | |
| 223 | OLIG1 (region 1; | 21 | 34442354- | oligodendrocyte transcription |
| | OLIG1_2354) | | 34442622 | factor 1 |
| 224 | OLIG1 (region 2; | 21 | 34444364- | oligodendrocyte transcription |
| | OLIG1_4364) | | 34444415 | factor 1 |
| 225 | PALLD | 4 | 169798991- | palladin, cytoskeletal associated |
| | | | 169799410 | protein |
| 226 | PAPLN | 14 | 73712777- | papilin, proteoglycan-like |
| | | | 73712896 | sulfated glycoprotein |
| 227 | PDLIM2 | 8 | 22437942- | PDZ and LIM domain 2 |
| | | | 22438142 | (mystique) |
| 228 | PKN1 | 19 | 14542944- | protein kinase N1 |
| | | | 14543000 | |
| 229 | PRDM14 | 8 | 70984345- | PR domain containing 14 |
| | | | 70984435 | |
| 230 | PRKG1 | 10 | 52751410- | protein kinase, cGMP-dependent, |
| | | | 52751477 | type I |
| 231 | PRMT7 | 16 | 68390257- | protein arginine methyltransferase |
| | | | 68390663 | 7 |
| 232 | PTGER2 | 14 | 52781719- | prostaglandin E receptor 2 |
| | | | 52782139 | (subtype EP2), 53 kDa |
| 233 | PTK2B | 8 | 27221308- | PTK2B protein tyrosine kinase 2 |
| | | | 27221453 | beta |
| 234 | RAD52 | 12 | 1059343- | RAD52 homolog (S. cerevisiae) |
| | | | 1059464 | |
| 235 | RBM38 | 20 | 55965125- | RNA binding motif protein 38 |
| | | | 55965344 | |
| 236 | RHOF | 12 | 122227754- | ras homolog gene family, |
| | | | 122227931 | member F (in filopodia) |
| 237 | RNF220 | 1 | 44873266- | ring finger protein 220 |
| | | | 44874033 | |
| 238 | RTN4RL1 | 17 | 1901564- | reticulon 4 receptor-like 1 |
| | | | 1901599 | |
| 239 | RXRA | 9 | 137288279- | retinoid X receptor, alpha |
| | | | 137288410 | |
| 240 | SDCCAG8 | 1 | 243646523- | serologically defined colon |
| | | | 243646579 | cancer antigen 8 |
| 241 | SHROOM1 | 5 | 132161402- | shroom family member 1 |
| | | | 132161696 | |
| 242 | SKI | 1 | 2232330- | v-ski sarcoma viral oncogene |
| | | | 2232617 | homolog (avian) |
| 243 | SLC12A8 | 3 | 124860570- | solute carrier family 12 |
| | | | 124860899 | (potassium/chloride transporters), |
| | | | | member 8 |
| 244 | SLC25A47 | 14 | 100784681- | solute carrier family 25, member |
| | | | 100784782 | 47 |
| 245 | SPEG (region 1; | 2 | 220299760- | SPEG complex locus |
| | SPEG_9760) | | 220299820 | |
| 246 | SPEG (region 2; | 2 | 220313572- | SPEG complex locus |
| | SPEG_3572) | | 220313655 | |
| 247 | SUCLG2 | 3 | 67706423- | succinate-CoA ligase, GDP- |
| | | | 67706568 | forming, beta subunit |
| 248 | TBC1D10C | 11 | 67176846- | TBC1 domain family, member |
| | | | 67177042 | 10C |
| 249 | TMEM132E | 17 | 32964596- | transmembrane protein 132E |
| | | | 32964694 | |
| 250 | VIPR2 | 7 | 158937914- | vasoactive intestinal peptide |
| | | | 158937982 | receptor 2 |
| 251 | WDR66 | 12 | 122356458- | WD repeat domain 66 |
| | | | 122356531 | |
| 252 | WNT6 | 2 | 219736286- | wingless-type MMTV integration |
| | | | 219736388 | site family, member 6 |
| 253 | ZDHHC18 | 1 | 27160118- | zinc finger, DHHC-type |
| | | | 27160221 | containing 18 |
| 254 | ZNF382 | 19 | 37096235- | zinc finger protein 382 |
| | | | 37096414 | |
| 255 | ZNF626 | 19 | 20844163- | zinc finger protein 626 |
| | | | 20844274 | |

### Example 2

Human papillomavirus associated oropharyngeal squamous cell carcinoma (HPV(+)OPSCC) rates continue to rise worldwide. Investigation into circulating tumor HPV DNA (ctHPVDNA) and pan cancer assays have been promising, but discriminatory data between anogenital and HPV(+)OPSCC is lacking. Thus, experiments were conducted to assess the feasibility of targeted assay of a panel of methylated DNA markers (MDMs) for detection OPSCC. A panel of methylation markers for human papillomavirus associated cervical squamous cell carcinoma (HPV(+)CSCC) was analyzed and validated for use as markers for HPV(+)OPSCC.

Patients meeting inclusion criteria had a primary (non-recurrent) tumor, had no prior history of pelvic or head and neck cancer or neoplasia, no exposure to chemotherapeutics within the last year, had no prior therapeutic radiation to the target region, had not had a transplant, had adequate clinical history on file and sufficient target tissue available (>5 mm), and were ≥18 years of age. Methylated DNA markers (MDMs) identified from sequenced differentially methylated regions were selected from a panel previously validated for HPV(+)OPSCC and evaluated on DNA from independent formalin-fixed, paraffin-embedded HPV(+)OPSCC, HPV(+)CSCC, normal oropharynx, and normal cervix tissue using methylation-specific polymerase chain reaction. White blood cells (WBCs) were used as a background control.

Thirty-four patients with HPV(+)OPSCC, 36 with HPV(+)CSCC, 26 with normal oropharyngeal tonsil tissue, and 24 with normal cervical tissue met inclusion criteria. The HPV(+)OPSCC was slightly older (57 vs 44, p=0.027), more frequent alcohol exposure (85% vs 64%, p=0.02), but similar ACE-27 comorbidity score (p=0.078) and tobacco use (p=0.066) compared to all others. Approximately 88% of the HPV(+)OPSCC and 58% of the normal tonsil patients were male. 0% of HPV(+)CSCC and 0% of normal cervical patients had a prior abnormal pap smear. Tumor stages were stage I (83% vs 47%), stage II (11% vs 44%) and stage III (6% vs 9%) for HPV(+)CSCC and HPV(+)OPSCC, respectively. Twenty-one MDM were evaluated and areas under the receiver operator characteristic curve (AUC) are reported for HPV(+)OPSCC and HPV(+)CSCC. Table 2 presents the 21 markers, the source of each marker, and the respective chromosome information. Table 3 presents related primer sequence information for the markers recited in Table 2.

TABLE 2
DMRs identifying HPV(+)OPSCC and HPV(+)CSCC, and the tissue
source and chromosome information for each respective region.

| DMR | | | Chromosome | Chromosome |
|---|---|---|---|---|
| No. | Gene Annotation | Source | No. | Coordinates |
| 1 | ABCB1 | Cervix | 7 | 87257074-87257239 |
| 2 | ARHGAP12 | Cervix | 10 | 32218791-32218902 |
| 3 | ASCL1 | Cervix | 12 | 103351856-103352059 |
| 4 | C1orf114_A (C1orf114_6696) | Cervix | 1 | 169396696-169396795 |
| 5 | EMX1 | Esophagus | 2 | 73147710-73147772 |
| 6 | GRIN2D | Esophagus | 19 | 48918144-48918350 |
| 7 | LOC645323 (region 3; | Esophagus | 5 | 87970706-87970894 |
| | LOC645323_0706) | | | |
| 8 | MAX.chr6.58147682-58147771 | Cervix | 6 | 58147682-58147771 |
| 9 | MAX.chr9.36739811-36739868 | Cervix | 9 | 36739811-36739868 |
| 10 | NEUROG3 | Cervix | 10 | 71332209-71332333 |
| 11 | NID2 | Cervix | 14 | 52535717-52536126 |
| 12 | TBX15 | Esophagus | 1 | 119527066-119527655 |
| 13 | TMEM200C | Cervix | 18 | 5890757-5890849 |
| 14 | TSPYL5 | Esophagus | 8 | 98289858-98290220 |
| 15 | TTYH1 | Cervix | 19 | 54926696-54926845 |
| 16 | VWC2 | Esophagus | 7 | 49813135-49814168 |
| 17 | ZNF610 | Esophagus | 19 | 52839503-52840013 |
| 18 | ZNF69 | Cervix | 19 | 11998671-11998972 |
| 19 | ZNF773 | Cervix | 19 | 58011327-58011598 |
| 20 | ZNF781_A (ZNF781_2950A) | Esophagus | 19 | 38182950-38183127 |
| 21 | ZNF781_B (ZNF781_2950B) | Cervix | 19 | 38182950-38183230 |

TABLE 3
DMRs identifying HPV(+)OPSCC and HPV(+)CSCC and their corresponding
primer sequences.

| DMR | | Forward Primer 5′-3′ | SEQ ID | Reverse Primer 5′-3′ | SEQ ID |
|---|---|---|---|---|---|
| No. | Gene Annotation | Sequence (hg19) | NO: | Sequence (hg19) | NO: |
| 1 | ABCB1 | CGCGAAAAGATTTTA | 1 | AACCCGCACCCGA | 2 |
| | | TATTGGTATTACGT | | AACTAACGAC | |
| 2 | ARHGAP12 | TCGTATTTTGTAAGGT | 3 | AACTCTTTCCTATA | 4 |
| | | ATAATTCGG | | CTTCACTTCGTA | |
| 3 | ASCL1 | TAGAGTTTTTTATGCG | 5 | ATTCTCTCTATATC | 6 |
| | | TAGCGGCGG | | CCCCTCGCGAA | |
| 4 | Clorf114_A | TTTTAATTACGAGAG | 7 | CCGAAATAAATACC | 8 |
| | (Clorf114_6696) | CGATAAAAATTTGCG | | GAAAAAAATCGAT | |
| | | T | | | |
| 5 | EMX1 | CGGGTTTTAGCGATG | 9 | CCTTTTCGTTCGTA | 10 |
| | | TTTATTTTAGTTTCGT | | TAAAATTTCGTT | |
| 6 | GRIN2D | TTTTTCGTTCGTTTTT | 11 | AACGCATACCATCG | 12 |
| | | TTATTTTTTCGA | | ACTTCAACTACGAC | |
| 7 | LOC645323 | GGTAGTGTTTTTTCGA | 13 | GATACGCGAACAC | 14 |
| | (region 3; | GACGCGGTC | | GCACACG | |
| | LOC645323_0706) | | | | |
| 8 | MAX.chr6.58147682- | TGTTTATGGATTTAG | 15 | AAAAAATACAACG | 16 |
| | 58147771 | GTGAGGACGG | | TTTAACCGCGAA | |
| 9 | MAX.chr9.36739811- | TGATAGGATGTTCGT | 17 | AAAAAACTACGCC | 18 |
| | 36739868 | TTAGTCGCGG | | GATCCCCGAA | |
| 10 | NEUROG3 | GGTCGTTTTTTTAGCG | 19 | CGAAAAACTAAAC | 20 |
| | | ACGCGGC | | AACCCAAACGAT | |
| 11 | NID2 | AGGAGTTTTTATTTCG | 21 | ATAACCACCACATC | 22 |
| | | TTAGTTTCGT | | TAATTCTCGTT | |
| 12 | TBX15 | CGCGTAGGTCGGATT | 23 | TTAATTAACGCCCG | 24 |
| | | GATTTTTCGT | | AACACCCGC | |
| 13 | TMEM200C | GAACGCGGTTTTTAG | 25 | CATACTACCCTCTA | 26 |
| | | GAGATTTCGA | | CGCCGCGAA | |
| 14 | TSPYL5 | TGGCGGCGGAGGTAG | 27 | TCGATCCCGACCGA | 28 |
| | | TTTTAAAGATAC | | AAACTAACGTC | |
| 15 | TTYH1 | TTTTTGTTTTTGGGGC | 29 | ATACATCTCCTCCA | 30 |
| | | GCGAAAAC | | CCAACTACCCCGC | |
| 16 | VWC2 | TTGGAGAGTTTTTCG | 31 | GAAAACCACCCTA | 32 |
| | | AATTTTTTCGC | | ACGCCG | |
| 17 | ZNF610 | ATTTGGTTTAAGGTA | 33 | TATATTAAAATCGC | 34 |
| | | GAACGAGCGG | | TCGACGACGAA | |
| 18 | ZNF69 | GGGGTTGTTTGGATC | 35 | CCCGCAAAAAAAC | 36 |
| | | GATCGGAATC | | CAAAATCTCGC | |
| 19 | ZNF773 | TATAGGTTTCGATGG | 37 | ATTCGAAAAAAAC | 38 |
| | | CGGCGGTTAC | | CGAAAAAACGCGA | |
| | | | | C | |
| 20 | ZNF781_A | TCGTTAGGAATTAGT | 39 | CGTTCTCTTATTTTC | 40 |
| | (ZNF781_2950A) | GGTTGGATTTATCGC | | CGAATACGCGAA | |
| 21 | ZNF781_B | TAGTTTATAAACGCG | 41 | CCAACGACTACTAA | 42 |
| | (ZNF781_2950B) | GCGGAATCGG | | ATCAAAAAACGCA | |

As shown in Table 4, within HPV(+)CSCC, 18/21 (86%) of MDMs achieved an AUC ≥0.9 and all MDMs exhibited better than chance classifications relative to control cervical tissue (all p<0.0001). For the HPV(+)OPSCC cohort, a majority of MDMs had lower AUCs when compared to AUCs for HPV(+)CSCC. However, 5/21 (24%) achieved an AUC ≥0.90, 15/21 (71%) achieved an AUC ≥0.8, and 19/21 (90%) exhibited better than chance classifications relative to control tonsil tissue (all p<0.001).

TABLE 4
DMRs identifying HPV(+)OPSCC and HPV(+)CSCC
and their corresponding AUCs and P-values.

| | | CSCC AUC | OPSCC AUC | AUC CSCC vs |
|---|---|---|---|---|
| DMR. | | (95% CI) | (95% CI) | AUC OPSCC |
| No. | Gene Annotation | P-value | P-value | P-value |
| 6 | GRIN2D | 0.72 (0.59-0.85) | 0.91 (0.83-0.99) | 0.019 |
| | | p = 0.0001 | p = 0.0001 | |
| 5 | EMX1 | 0.93 (0.86-0.99) | 0.77 (0.65-0.89) | 0.0191 |
| | | p = 0.0001 | p = 0.0001 | |
| 16 | VWC2 | 0.88 (0.79-0.97) | 0.57 (0.42-0.72) | 0.0001 |
| | | p = 0.0001 | p = 0.173 | |
| 17 | ZNF610 | 0.99 (0.98-1) | 0.89 (0.8-0.97) | 0.0162 |
| | | p = 0.0001 | p = 0.0001 | |
| 20 | ZNF781_A (ZNF781_2950A) | 0.92 (0.86-0.99) | 0.79 (0.67-0.91) | 0.0585 |
| | | p = 0.0001 | p = 0.0001 | |
| 12 | TBX15 | 0.99 (0.96-1) | 0.82 (0.72-0.93) | 0.0025 |
| | | p = 0.0001 | p = 0.0001 | |
| 14 | TSPYL5 | 0.94 (0.87-1) | 0.82 (0.72-0.93) | 0.058 |
| | | p = 0.0001 | p = 0.0001 | |
| 7 | LOC645323 (region 3; | 0.95 (0.89-1) | 0.88 (0.8-0.97) | 0.1952 |
| | LOC645323_0706) | p = 0.0001 | p = 0.0001 | |
| 3 | ASCL1 | 0.98 (0.93-1) | 0.86 (0.76-0.95) | 0.0187 |
| | | p = 0.0001 | p = 0.0001 | |
| 1 | ABCB1 | 0.97 (0.93-1) | 0.74 (0.61-0.86) | 0.0001 |
| | | p = 0.0001 | p = 0.0001 | |
| 18 | ZNF69 | 0.89 (0.8-0.97) | 0.95 (0.9-1) | 0.2017 |
| | | p = 0.0001 | p = 0.0001 | |
| 9 | MAX.chr9.36739811-36739868 | 0.97 (0.92-1) | 0.82 (0.72-0.93) | 0.0113 |
| | | p = 0.0001 | p = 0.0001 | |
| 2 | ARHGAP12 | 0.91 (0.83-0.99) | 0.9 (0.81-1) | 0.9223 |
| | | p = 0.0001 | p = 0.0001 | |
| 4 | C1orf114_A (C1orf114_6696) | 0.94 (0.86-1) | 0.88 (0.8-0.97) | 0.3366 |
| | | p = 0.0001 | p = 0.0001 | |
| 8 | MAX.chr6.58147682-58147771 | 0.99 (0.97-1) | 0.86 (0.76-0.96) | 0.0099 |
| | | p = 0.0001 | p = 0.0001 | |
| 10 | NEUROG3 | 0.95 (0.89-1) | 0.7 (0.57-0.83) | 0.0001 |
| | | p = 0.0001 | p = 0.0015 | |
| 11 | NID2 | 0.98 (0.95-1) | 0.86 (0.77-0.95) | 0.0207 |
| | | p = 0.0001 | p = 0.0001 | |
| 13 | TMEM200C | 0.95 (0.89-1) | 0.54 (0.39-0.69) | 0.0001 |
| | | p = 0.0001 | p = 0.2853 | |
| 15 | TTYH1 | 0.99 (0.98-1) | 0.82 (0.71-0.93) | 0.0023 |
| | | p = 0.0001 | p = 0.0001 | |
| 19 | ZNF773 | 0.93 (0.86-1) | 0.9 (0.82-0.98) | 0.5696 |
| | | p = 0.0001 | p = 0.0001 | |
| 21 | ZNF781_B (ZNF781_2950B) | 1 (1-1) | 0.92 (0.86-0.99) | 0.0198 |
| | | p = 0.0001 | p = 0.0001 | |

Patient characteristics for the experimental samples described above are provided in Table 5.

TABLE 5
Patient characteristics.

| | CSCC | | NCS | | NOP | | OPSCC | | |
|---|---|---|---|---|---|---|---|---|---|
| | (N = 36) | | (N = 24) | | (N = 26) | | (N = 34) | | p value |
| Age | | | | | | | | | 0.027 |
| Median (Q1, Q3) | 43 | (37, 53) | 44 | (38, 55) | 45 | (33, 66) | 57 | (48, 62) | |
| Sex | | | | | | | | | <0.001 |
| Female | 36 | (100.0%) | 24 | (100.0%) | 11 | (42.3%) | 4 | (11.8%) | |
| Male | 0 | (0.0%) | 0 | (0.0%) | 15 | (57.7%) | 30 | (88.2%) | |
| Race | | | | | | | | | <0.001 |
| non-White | 10 | (27.8%) | 0 | (0.0%) | 1 | (3.8%) | 1 | (2.9%) | |
| White | 26 | (72.2%) | 24 | (100.0%) | 25 | (96.2%) | 33 | (97.1%) | |
| Alcohol Use | | | | | | | | | 0.020 |
| N-Miss | 16 | | 1 | | 2 | | 1 | | |
| No | 11 | (55.0%) | 7 | (30.4%) | 6 | (25.0%) | 5 | (15.2%) | |
| Yes | 9 | (45.0%) | 16 | (69.6%) | 18 | (75.0%) | 28 | (84.8%) | |
| Tobacco Use | | | | | | | | | 0.066 |
| N-Miss | 2 | | 1 | | 0 | | 0 | | |
| No | 11 | (32.4%) | 11 | (47.8%) | 17 | (65.4%) | 19 | (55.9%) | |
| Yes | 23 | (67.6%) | 12 | (52.2%) | 9 | (34.6%) | 15 | (44.1%) | |
| ACE-27 | | | | | | | | | 0.078 |
| 1 | 19 | (52.8%) | 5 | (20.8%) | 8 | (30.8%) | 17 | (50.0%) | |
| 2 | 9 | (25.0%) | 15 | (62.5%) | 8 | (30.8%) | 8 | (23.5%) | |
| 3 | 6 | (16.7%) | 3 | (12.5%) | 8 | (30.8%) | 7 | (20.6%) | |
| 4 | 2 | (5.6%) | 1 | (4.2%) | 2 | (7.7%) | 2 | (5.9%) | |
| Positive PAP | | | | | | | | | |
| N-Miss | 35 | | 15 | | 21 | | 34 | | |
| ASCUS | 0 | (0.0%) | 0 | (0.0%) | 1 | (20.0%) | 0 | | |
| Negative | 1 | (100.0%) | 9 | (100.0%) | 4 | (80.0%) | 0 | | |
| Cancer Stage | | | | | | | | | |

| N-Miss | 0 | | 24 | 26 | 0 | |
|---|---|---|---|---|---|---|
| I | 30 | (83.3%) | 0 | 0 | 16 | (47.1%) |
| II | 4 | (11.1%) | 0 | 0 | 15 | (44.1%) |
| III | 2 | (5.6%) | 0 | 0 | 3 | (8.8%) |

In accordance with the above data, the following materials and method were used.

Samples. Up to 10 (10 um)/2 (2 mm) FFPE tissue cores of the area of interest were obtained from each tissue block. Smaller sizes of tissue cores were obtained from multiple tissue blocks dependent upon the tissue size of the block. At least two 2 mm tissue cores or ten slides of 10 um were needed to get a sufficient quality DNA because DNA from FFPE tissues quality is usually low and DNA is fragmented. Using core punches preserves more tissue on the block since only the small area of interest will be taken rather than many sections of the whole block. In addition, a slide will be P16 stained for HPV testing. All tissue samples were reviewed by a Mayo Clinic pathologist to confirm histology. Quantitative methylation specific PCR assays (qMSP) have already been developed for top cancer tissue specific marker candidates from prior work. These markers were validated in target tissues from patients groups identified above. Tissues were macro-dissected and histology reviewed by an expert GI pathologist. Samples were age sex matched, randomized, and blinded. DNA from was purified using the QIAamp DNA FFPE Tissue kit (FFPE tissues) and QIAamp DNA Blood Mini kit (buffy coat samples) (Qiagen, Valencia CA). DNA was re-purified with AMPure XP beads (Beckman-Coulter, Brea CA) and quantified by PicoGreen (Thermo-Fisher, Waltham MA). DNA integrity was assessed using qPCR.

Biomarker Selection. CSCC biomarkers which were previously identified and validated were chosen to test the OPSCC sample cohorts. Thirteen methylated DNA markers (MDMs) in independent sample sets had been able to discriminate cancer from normal cervical tissue with individual “area under the ROC curve” (AUC) performance in excess of 0.90 and at least 5-fold methylation differentials. In addition, 8 MDMs were included-identified from earlier pan-GI discovery studies—which had demonstrated high levels of hypermethylation in esophageal squamous cell cancers. These 8 were later tested in a small cohort of head and neck cancers and compared to normal esophageal epithelium—and were shown to be highly methylated in these cancers as well (see, e.g., Table 2).

Biomarker Testing. Up to 300 ng of sample DNA was treated with sodium bisulfite and re-purified using the Zymo EZ DNA Methylation method (Zymo Research, Irvine CA). Quantitative methylation specific PCR (qMSP) assays with oligos specific to differentially methylated CpGs were run on the converted DNA. Approximately 10 ng of converted DNA (per marker) was amplified using SYBR Green detection on Roche 480 LightCyclers (Roche, Basel Switzerland). Serially diluted universal methylated genomic DNA (Zymo Research) was used as a quantitation standard. A CpG agnostic ACTB (β-actin) assay was used as an input reference and normalization control. Results were expressed as methylated copies (specific marker)/copies of ACTB.

Statistics. Descriptive statistics were utilized to broadly analyze the sample data. These experiments were conducted to evaluate the similarity in methylation markers between CSCC and OPSCC patients. Initial analysis of variance between the two groups was performed with a student's t-test.

### Example 3

In this Example, experiments were conducted to identify additional DMRs capable of distinguishing oropharyngeal cancer (e.g., OPSCC) from control samples (e.g., tissue and buffy coat controls).

A proprietary methodology of sample preparation, sequencing, analyses pipelines, and filters were utilized to identify and narrow differentially methylated regions (DMRs) to those which would pinpoint these oropharyngeal cancers and excel in a clinical testing environment. From the tissue-to-tissue analysis, 129 hypermethylated OPSCC DMRs were identified (Table 1 above; Table 6 below). They included OPSCC specific regions as well as regions that are frequently methylated in several or more epithelial cancer types. The OPSCC tissue to buffy coat analysis yielded 105 hypermethylated tissue DMRs with AUC's >0.95 and less than 1% noise in leukocytes (Table 1 above; Table 7 below).

TABLE 6
Methylated regions distinguishing oropharyngeal cancer (OSPCC) from controls (e.g., tonsil tissue controls).

| DMR | | Chromosome | Chromosome | |
|---|---|---|---|---|
| No. | Gene Annotation | No. | Coordinates | Gene Title |
| 22 | ALX4 | 11 | 44326332-44326444 | ALX homeobox 4 |
| 23 | ATP10A | 15 | 26108236-26108573 | ATPase, class V, type 10A |
| 24 | C1orf114_B (C1orf114_6622) | 1 | 169396622-169396765 | chromosome 1 open reading |
| | | | | frame 114 |
| 25 | C1QL3 | 10 | 16562329-16562405 | complement component 1, q |
| | | | | subcomponent-like 3 |
| 26 | CA8 | 8 | 61193337-61193995 | carbonic anhydrase VIII |
| 27 | CACNA1A | 19 | 13617008-13617106 | calcium channel, voltage- |
| | | | | dependent, P/Q type, alpha 1A |
| | | | | subunit |
| 28 | CACNG8 | 19 | 54485837-54485846 | calcium channel, voltage- |
| | | | | dependent, gamma subunit 8 |
| 29 | CALCA | 11 | 14995266-14995572 | calcitonin-related polypeptide |
| | | | | alpha |
| 30 | CCNA1 | 13 | 37006107-37006678 | cyclin A1 |
| 31 | CLIC6 | 21 | 36041893-36041956 | chloride intracellular |
| | | | | channel 6 |
| 32 | CLSTN2 | 3 | 139653792-139654257 | calsyntenin 2 |
| 33 | CR1 | 1 | 207669489-207669878 | complement component (3b/4b) |
| | | | | receptor 1 (Knops blood group) |
| 34 | CTNND2 | 5 | 11904006-11904366 | catenin (cadherin-associated |
| | | | | protein), delta 2 (neural |
| | | | | plakophilin-related arm-repeat |
| | | | | protein) |
| 35 | DAB1 | 1 | 58715813-58716179 | disabled homolog 1 (Drosophila) |
| 36 | DGKG | 3 | 186079645-186079768 | diacylglycerol kinase, gamma |
| | | | | 90 kDa |
| 37 | DOK1 | 2 | 74782446-74782597 | docking protein 1, 62 kDa |
| | | | | (downstream of tyrosine kinase 1) |
| 38 | DOK6 | 18 | 67068039-67068223 | docking protein 6 |
| 39 | DPP4 | 2 | 162930234-162930577 | dipeptidyl-peptidase 4 |
| 40 | DUXA | 19 | 57683241-57683288 | double homeobox A |
| 41 | ELMO1 (region 1; ELMO1_7417) | 7 | 37487417-37488625 | engulfment and cell motility 1 |
| 42 | EMBP1 | 1 | 121260881-121261189 | embigin pseudogene 1 |
| 43 | EPDR1 (region 1; EPDR1_0389) | 7 | 37960389-37960975 | ependymin related protein 1 |
| | | | | (zebrafish) |
| 44 | FGF12 | 3 | 192125977-192126052 | fibroblast growth factor 12 |
| 45 | FLJ43390 | 14 | 62583913-62584109 | hypothetical LOC646113 |
| 46 | FMN2 | 1 | 240255182-240255338 | formin 2 |
| 47 | FOXB2 | 9 | 79633392-79633419 | forkhead box B2 |
| 48 | FOXD4 | 9 | 117499-117614 | forkhead box D4 |
| 49 | FREM3 | 4 | 144621581-144621670 | FRASI related extracellular |
| | | | | matrix 3 |
| 50 | GALR1 | 18 | 74962243-74962454 | galanin receptor 1 |
| 51 | GDF6 | 8 | 97171935-97172016 | growth differentiation factor 6 |
| 52 | GFRA1 | 10 | 118031547-118031694 | GDNF family receptor alpha 1 |
| 53 | GRIK3 | 1 | 37498816-37498828 | glutamate receptor, ionotropic, |
| | | | | kainate 3 |
| 54 | HOXB3 | 17 | 46655387-46656122 | homeobox B3 |
| 55 | HOXB4 | 17 | 46659595-46659703 | homeobox B4 |
| 56 | HPSE2 | 10 | 100993583-100993598 | heparanase 2 |
| 57 | LDLRAD2 | 1 | 22140984-22141213 | low density lipoprotein receptor |
| | | | | class A domain containing 2 |
| 58 | LHX2 | 9 | 126780041-126780896 | LIM homeobox 2 |
| 59 | LOC100131366 | 14 | 103655316-103655503 | hypothetical LOC100131366 |
| 60 | LOC345643 | 5 | 54516736-54516809 | hypothetical LOC345643 |
| 61 | LOC386758 | 19 | 56904686-56905053 | — |
| 62 | LOC645323 (region 1; | 5 | 87956761-87957004 | hypothetical LOC645323 |
| | LOC645323_6761) | | | |
| 63 | LOC645323 (region 2; | 5 | 87971583-87971822 | hypothetical LOC645323 |
| | LOC645323_1583) | | | |
| 64 | LOC648809 | 15 | 84748956-84749041 | elongation factor Tu GTP-binding |
| | | | | domain-containing protein 1 |
| | | | | pseudogene |
| 65 | LOC728392 | 17 | 5404375-5404508 | hypothetical protein LOC728392 |
| 66 | MAML3 | 4 | 140656525-140656723 | mastermind-like 3 (Drosophila) |
| 67 | MAPRE2 | 18 | 32557478-32557725 | microtubule-associated protein, |
| | | | | RP/EB family, member 2 |
| 68 | MAX.chr1.226288154-226288189 | 1 | 226288154-226288189 | — |
| 69 | MAX.chr1.2375078-2375126 | 1 | 2375078-2375126 | — |
| 70 | MAX.chr1.241587339-241587784 | 1 | 241587339-241587784 | — |
| 71 | MAX.chr1.50798781-50799423 | 1 | 50798781-50799423 | — |
| 72 | MAX.chr10.22765150-22765477 | 10 | 22765150-22765477 | — |
| 73 | MAX.chr10.23462342-23462436 | 10 | 23462342-23462436 | — |
| 74 | MAX.chr11.14926602-14927044 | 11 | 14926602-14927044 | — |
| 75 | MAX.chr11.58903531-58903592 | 11 | 58903531-58903592 | — |
| 76 | MAX.chr13.28527984-28528214 | 13 | 28527984-28528214 | — |
| 77 | MAX.chr13.29106641-29107037 | 13 | 29106641-29107037 | — |
| 78 | MAX.chr14.100784488-100784782 | 14 | 100784488-100784782 | — |
| 79 | MAX.chr16.3221176-3221223 | 16 | 3221176-3221223 | — |
| 80 | MAX.chr16.3222040-3222098 | 16 | 3222040-3222098 | — |
| 81 | MAX.chr16.71460171-71460282 | 16 | 71460171-71460282 | — |
| 82 | MAX.chr19.11805263-11805639 | 19 | 11805263-11805639 | — |
| 83 | MAX.chr19.16394457-16394646 | 19 | 16394457-16394646 | — |
| 84 | MAX.chr19.21657626-21657769 | 19 | 21657626-21657769 | — |
| 85 | MAX.chr19.22034646-22034887 | 19 | 22034646-22034887 | — |
| 86 | MAX.chr19.23299989-23300156 | 19 | 23299989-23300156 | — |
| 87 | MAX.chr19.30713427-30713588 | 19 | 30713427-30713588 | — |
| 88 | MAX.chr19.30716926-30717074 | 19 | 30716926-30717074 | — |
| 89 | MAX.chr19.30718373-30719719 | 19 | 30718373-30719719 | — |
| 90 | MAX.chr2.118981724-118982174 | 2 | 118981724-118982174 | — |
| 91 | MAX.chr2.127783107-127783403 | 2 | 127783107-127783403 | — |
| 92 | MAX.chr2.173099712-173099791 | 2 | 173099712-173099791 | — |
| 93 | MAX.chr2.66808635-66808731 | 2 | 66808635-66808731 | — |
| 94 | MAX.chr22.50064113-50064259 | 22 | 50064113-50064259 | — |
| 95 | MAX.chr3.137489884-137490061 | 3 | 137489884-137490061 | — |
| 96 | MAX.chr5.138923141-138923219 | 5 | 138923141-138923219 | — |
| 97 | MAX.chr5.42995180-42995535 | 5 | 42995180-42995535 | — |
| 98 | MAX.chr6.38683091-38683226 | 6 | 38683091-38683226 | — |
| 99 | MAX.chr7.121952014-121952084 | 7 | 121952014-121952084 | — |
| 100 | MAX.chr7.155166980-155167310 | 7 | 155166980-155167310 | — |
| 101 | MAX.chr8.99986792-99986864 | 8 | 99986792-99986864 | — |
| 102 | MAX.chr9.79627078-79627116 | 9 | 79627078-79627116 | — |
| 103 | MAX.chr9.79638034-79638077 | 9 | 79638034-79638077 | — |
| 104 | MAX.chr9.98789824-98789847 | 9 | 98789824-98789847 | — |
| 105 | MDFI | 6 | 41606290-41606487 | MyoD family inhibitor |
| 106 | MECOM | 3 | 169376578-169376702 | MDS1 and EVI1 complex locus |
| 107 | MED12L | 3 | 150804663-150805021 | mediator complex subunit 12-like |
| 108 | MIR129-2 | 11 | 43602934-43603032 | microRNA 129-2 |
| 109 | MIR196A1 | 17 | 46711335-46711359 | microRNA 196a-1 |
| 110 | NELL1 | 11 | 20691446-20691520 | NEL-like 1 (chicken) |
| 111 | NPY | 7 | 24323248-24323319 | neuropeptide Y |
| 112 | ONECUT2 | 18 | 55105693-55105794 | one cut homeobox 2 |
| 113 | OPCML | 11 | 132952679-132952966 | opioid binding protein/cell |
| | | | | adhesion molecule-like |
| 114 | PARP15 | 3 | 122296579-122296700 | poly (ADP-ribose) polymerase |
| | | | | family, member 15 |
| 115 | PDGFD | 11 | 104034648-104035133 | platelet derived growth factor D |
| 116 | PEX5L | 3 | 179754874-179754929 | peroxisomal biogenesis |
| | | | | factor 5-like |
| 117 | PRR15 | 7 | 29605829-29606153 | proline rich 15 |
| 118 | SEMA6A | 5 | 115912116-115912302 | sema domain, transmembrane |
| | | | | domain (TM), and cytoplasmic |
| | | | | domain, (semaphorin) 6A |
| 119 | SFMBT2 | 10 | 7450191-7450327 | Scm-like with four mbt domains 2 |
| 120 | SGIP1 | 1 | 66998873-66999034 | SH3-domain GRB2-like |
| | | | | (endophilin) interacting protein 1 |
| 121 | SIM2 | 21 | 38120049-38120250 | single-minded homolog 2 |
| | | | | (Drosophila) |
| 122 | SLC35F3 | 1 | 234040507-234040529 | solute carrier family 35, |
| | | | | member F3 |
| 123 | SLCO4C1 | 5 | 101632236-101632315 | solute carrier organic anion |
| | | | | transporter family, member 4C1 |
| 124 | SORCS3 (region 1; SORCS3_0952) | 10 | 106400952-106401370 | sortilin-related VPS10 domain |
| | | | | containing receptor 3 |
| 125 | SORCS3 (region 2; SORCS3_2558) | 10 | 106402558-106402652 | sortilin-related VPS10 domain |
| | | | | containing receptor 3 |
| 126 | ST6GALNAC5 | 1 | 77333925-77334022 | ST6 (alpha-N-acetyl-neuraminyl- |
| | | | | 2,3-beta-galactosyl-1,3)-N- |
| | | | | acetylgalactosaminide alpha-2,6- |
| | | | | sialyltransferase 5 |
| 127 | ST8SIA5 | 18 | 44336443-44337094 | ST8 alpha-N-acetyl-neuraminide |
| | | | | alpha-2,8-sialyltransferase 5 |
| 128 | SV2C | 5 | 75379004-75379253 | synaptic vesicle glycoprotein 2C |
| 129 | TACC2 | 10 | 123923132-123923269 | transforming, acidic coiled-coil |
| | | | | containing protein 2 |
| 130 | TFAP2E | 1 | 36043354-36043427 | transcription factor AP-2 epsilon |
| | | | | (activating enhancer binding |
| | | | | protein 2 epsilon) |
| 131 | TLX2 | 2 | 74741063-74741295 | T-cell leukemia homeobox 2 |
| 132 | TLX3 | 5 | 170738892-170739145 | T-cell leukemia homeobox 3 |
| 133 | TRH | 3 | 129693355-129693854 | thyrotropin-releasing hormone |
| 134 | TRIM58 | 1 | 248020543-248021004 | tripartite motif-containing 58 |
| 135 | VAV3 | 1 | 108508521-108508601 | vav 3 guanine nucleotide |
| | | | | exchange factor |
| 136 | VSTM2B (region 1; VSTM2B_6096) | 19 | 30016096-30016161 | V-set and transmembrane domain |
| | | | | containing 2B |
| 137 | VSTM2B (region 2; VSTM2B_7171) | 19 | 30017171-30018338 | V-set and transmembrane domain |
| | | | | containing 2B |
| 138 | WDR17 | 4 | 176987103-176987241 | WD repeat domain 17 |
| 139 | ZNF254 | 19 | 24269958-24270038 | zinc finger protein 254 |
| 140 | ZNF43 | 19 | 22018638-22019004 | zinc finger protein 43 |
| 141 | ZNF486 | 19 | 20278275-20278343 | zinc finger protein 486 |
| 142 | ZNF491 | 19 | 11909233-11909959 | zinc finger protein 491 |
| 143 | ZNF518B | 4 | 10463044-10463098 | zinc finger protein 518B |
| 144 | ZNF542 | 19 | 56879626-56879699 | zinc finger protein 542 |
| 145 | ZNF625 | 19 | 12267145-12267309 | zinc finger protein 625 |
| 146 | ZNF665 (region 1; ZNF665_6497) | 19 | 53696497-53696704 | zinc finger protein 665 |
| 147 | ZNF665 (region 2; ZNF665_0173) | 19 | 53700173-53700459 | zinc finger protein 665 |
| 148 | ZNF671 | 19 | 58238740-58239004 | zinc finger protein 671 |
| 149 | ZNF763 | 19 | 12075727-12076257 | zinc finger protein 763 |
| 150 | ZNF844 | 19 | 12175505-12176056 | zinc finger protein 844 |

TABLE 7
Methylated regions distinguishing oropharyngeal cancer (OSPCC)
from controls (e.g., normal buffy coat controls).

| DMR | | Chromosome | Chromosome | |
|---|---|---|---|---|
| No. | Gene Annotation | No. | Coordinates | Gene Title |
| 151 | AGRN | 1 | 969518-969570 | agrin |
| 152 | ANKRD35 | 1 | 145562791-145562917 | ankyrin repeat domain 35 |
| 153 | ARHGAP27 | 17 | 43487568-43487675 | Rho GTPase activating protein 27 |
| 154 | ARHGAP30 | 1 | 161039379-161039594 | Rho GTPase activating protein 30 |
| 155 | BCL2L11 | 2 | 111876417-111877177 | BCL2-like 11 (apoptosis |
| | | | | facilitator) |
| 156 | BIN2 | 12 | 51717948-51718130 | bridging integrator 2 |
| 157 | C10orf114 | 10 | 21789269-21789327 | chromosome 10 open reading |
| | | | | frame 114 |
| 158 | C4orf31 | 4 | 121992389-121992492 | chromosome 4 open reading |
| | | | | frame 31 |
| 159 | C6orf132 | 6 | 42072052-42072400 | chromosome 6 open reading |
| | | | | frame 132 |
| 160 | C6orf186 | 6 | 110679348-110679376 | chromosome 6 open reading |
| | | | | frame 186 |
| 161 | CCDC88B | 11 | 64108181-64108246 | coiled-coil domain containing |
| | | | | 88B |
| 162 | CRHBP | 5 | 76249579-76249699 | corticotropin releasing hormone |
| | | | | binding protein |
| 163 | DAPK1 | 9 | 90113698-90113817 | death-associated protein kinase 1 |
| 164 | DNMT3A | 2 | 25499945-25500298 | DNA (cytosine-5-)- |
| | | | | methyltransferase 3 alpha |
| 165 | DPP10 | 2 | 115919021-115919041 | dipeptidyl-peptidase 10 (non- |
| | | | | functional) |
| 166 | ELMO1 (region 2; ELMO1_2953) | 7 | 37392953-37393050 | engulfment and cell motility 1 |
| 167 | EPDR1 (region 2; EPDR1_6117) | 7 | 37956117-37956303 | ependymin related protein 1 |
| 168 | FAM19A2 | 12 | 62584870-62585101 | family with sequence similarity |
| | | | | 19 (chemokine (C-C motif)-like), |
| | | | | member A2 |
| 169 | FLJ45983 | 10 | 8097589-8098039 | hypothetical LOC399717 |
| 170 | FOSL 1 | 11 | 65661516-65661709 | FOS-like antigen 1 |
| 171 | FOXB1 | 15 | 60296761-60296789 | forkhead box B1 |
| 172 | GREM1 | 15 | 33011110-33011126 | gremlin 1 |
| 173 | HMHA1 | 19 | 1068372-1068513 | histocompatibility (minor) HA-1 |
| 174 | HOXA9 | 7 | 27205107-27205214 | homeobox A9 |
| 175 | IFFO1 | 12 | 6665226-6665425 | intermediate filament family |
| | | | | orphan 1 |
| 176 | INPP4B | 4 | 143767040-143767086 | inositol polyphosphate-4- |
| | | | | phosphatase, type II, 105 kDa |
| 177 | ITGB2 | 21 | 46340400-46340636 | integrin, beta 2 (complement |
| | | | | component 3 receptor 3 and 4 |
| | | | | subunit) |
| 178 | ITGB4 | 17 | 73749619-73750129 | integrin, beta 4 |
| 179 | ITPKB | 1 | 226862915-226863021 | inositol 1,4,5-trisphosphate 3- |
| | | | | kinase B |
| 180 | KCNIP2 | 10 | 103603570-103603685 | Kv channel interacting protein 2 |
| 181 | KLHDC7B | 22 | 50986987-50987042 | kelch domain containing 7B |
| 182 | LAT | 16 | 28996171-28996363 | linker for activation of T cells |
| 183 | LHX6 | 9 | 124981702-124981939 | LIM homeobox 6 |
| 184 | LIMK1 | 7 | 73508970-73509149 | LIM domain kinase 1 |
| 185 | LOC100128239 | 11 | 133906819-133906985 | hypothetical LOC100128239 |
| 186 | LOC100192379 | 4 | 122686355-122686433 | hypothetical LOC100192379 |
| 187 | LOC646278 | 15 | 29077633-29077650 | hypothetical LOC646278 |
| 188 | MAP2K2 | 19 | 4108818-4108937 | mitogen-activated protein |
| | | | | kinase 2 |
| 189 | MAX.chr1.210426156-210426257 | 1 | 210426156-210426257 | — |
| 190 | MAX.chr1.84326495-84326656 | 1 | 84326495-84326656 | — |
| 191 | MAX.chr10.119312785-119312882 | 10 | 119312785-119312882 | — |
| 192 | MAX.chr15.67326025-67326060 | 15 | 67326025-67326060 | — |
| 193 | MAX.chr16.54316401-54316453 | 16 | 54316401-54316453 | — |
| 194 | MAX.chr16.85482306-85482494 | 16 | 85482306-85482494 | — |
| 195 | MAX.chr17.74994454-74994572 | 17 | 74994454-74994572 | — |
| 196 | MAX.chr17.76339840-76339972 | 17 | 76339840-76339972 | — |
| 197 | MAX.chr2.7571082-7571136 | 2 | 7571082-7571136 | — |
| 198 | MAX.chr21.45577347-45577679 | 21 | 45577347-45577679 | — |
| 199 | MAX.chr3.14852538-14852568 | 3 | 14852538-14852568 | — |
| 200 | MAX.chr3.187676564-187676668 | 3 | 187676564-187676668 | — |
| 201 | MAX.chr4.174430662-174430793 | 4 | 174430662-174430793 | — |
| 202 | MAX.chr5.177411809-177411836 | 5 | 177411809-177411836 | — |
| 203 | MAX.chr6.45631561-45631625 | 6 | 45631561-45631625 | — |
| 204 | MAX.chr7.25892382-25892451 | 7 | 25892382-25892451 | — |
| 205 | MAX.chr7.402563-402641 | 7 | 402563-402641 | — |
| 206 | MAX.chr7.64349554-64349606 | 7 | 64349554-64349606 | — |
| 207 | MAX.chr8.142046239-142046398 | 8 | 142046239-142046398 | — |
| 208 | MAX.chr8.145900842-145901246 | 8 | 145900842-145901246 | — |
| 209 | MAX.chr9.126101804-126101848 | 9 | 126101804-126101848 | — |
| 210 | MAX.chr9.126978999-126979182 | 9 | 126978999-126979182 | — |
| 211 | MAX.chr9.36458633-36458725 | 9 | 36458633-36458725 | — |
| 212 | MAX.chr9.87905315-87905326 | 9 | 87905315-87905326 | — |
| 213 | MBP | 18 | 74818442-74818536 | myelin basic protein |
| 214 | MFNG | 22 | 37881211-37881422 | MFNG O-fucosylpeptide 3-beta- |
| | | | | N-acetylglucosaminyltransferase |
| 215 | MT1A (region 1; MT1A_9553) | 16 | 56669553-56669589 | metallothionein 1A |
| 216 | MT1A (region 2; MT1A_2245) | 16 | 56672245-56672278 | metallothionein 1A |
| 217 | MT1IP | 16 | 56709938-56709985 | metallothionein 1I (pseudogene) |
| 218 | NCOR2 (region 1; NCOR2_1781) | 12 | 124941781-124941955 | nuclear receptor corepressor 2 |
| 219 | NCOR2 (region 2; V NCOR2_0925) | 12 | 124950925-124950997 | nuclear receptor corepressor 2 |
| 220 | NFATC1 | 18 | 77159477-77159778 | nuclear factor of activated T- |
| | | | | cells, cytoplasmic, calcineurin- |
| | | | | dependent 1 |
| 221 | NKX3-2 | 4 | 13543800-13543846 | NK3 homeobox 2 |
| 222 | NRN1 | 6 | 6004182-6004237 | neuritin 1 |
| 223 | OLIG1 (region 1; OLIG1_354) | 21 | 34442354-34442622 | oligodendrocyte transcription |
| | | | | factor 1 |
| 224 | OLIG1 (region 2; OLIG1_4364) | 21 | 34444364-34444415 | oligodendrocyte transcription |
| | | | | factor 1 |
| 225 | PALLD | 4 | 169798991-169799410 | palladin, cytoskeletal |
| | | | | associated protein |
| 226 | PAPLN | 14 | 73712777-73712896 | papilin, proteoglycan-like |
| | | | | sulfated glycoprotein |
| 227 | PDLIM2 | 8 | 22437942-22438142 | PDZ and LIM domain 2 |
| | | | | (mystique) |
| 228 | PKN1 | 19 | 14542944-14543000 | protein kinase N1 |
| 229 | PRDM14 | 8 | 70984345-70984435 | PR domain containing 14 |
| 230 | PRKG1 | 10 | 52751410-52751477 | protein kinase, cGMP-dependent, |
| | | | | type I |
| 231 | PRMT7 | 16 | 68390257-68390663 | protein arginine methyl- |
| | | | | transferase 7 |
| 232 | PTGER2 | 14 | 52781719-52782139 | prostaglandin E receptor 2 |
| | | | | (subtype EP2), 53 kDa |
| 233 | PTK2B | 8 | 27221308-27221453 | PTK2B protein tyrosine kinase 2 |
| | | | | beta |
| 234 | RAD52 | 12 | 1059343-1059464 | RAD52 homolog (S. cerevisiae) |
| 235 | RBM38 | 20 | 55965125-55965344 | RNA binding motif protein 38 |
| 236 | RHOF | 12 | 122227754-122227931 | ras homolog gene family, |
| | | | | member F (in filopodia) |
| 237 | RNF220 | 1 | 44873266-44874033 | ring finger protein 220 |
| 238 | RTN4RL1 | 17 | 1901564-1901599 | reticulon 4 receptor-like 1 |
| 239 | RXRA | 9 | 137288279-137288410 | retinoid X receptor, alpha |
| 240 | SDCCAG8 | 1 | 243646523-243646579 | serologically defined colon |
| | | | | cancer antigen 8 |
| 241 | SHROOM1 | 5 | 132161402-132161696 | shroom family member 1 |
| 242 | SKI | 1 | 2232330-2232617 | v-ski sarcoma viral oncogene |
| | | | | homolog (avian) |
| 243 | SLC12A8 | 3 | 124860570-124860899 | solute carrier family 12 |
| | | | | (potassium/chloride |
| | | | | transporters), member 8 |
| 244 | SLC25A47 | 14 | 100784681-100784782 | solute carrier family 25, |
| | | | | member 47 |
| 245 | SPEG (region 1; SPEG_9760) | 2 | 220299760-220299820 | SPEG complex locus |
| 246 | SPEG (region 2; SPEG_3572) | 2 | 220313572-220313655 | SPEG complex locus |
| 247 | SUCLG2 | 3 | 67706423-67706568 | succinate-CoA ligase, GDP- |
| | | | | forming, beta subunit |
| 248 | TBC1D10C | 11 | 67176846-67177042 | TBC1 domain family, |
| | | | | member 10C |
| 249 | TMEM132E | 17 | 32964596-32964694 | transmembrane protein 132E |
| 250 | VIPR2 | 7 | 158937914-158937982 | vasoactive intestinal peptide |
| | | | | receptor 2 |
| 251 | WDR66 | 12 | 122356458-122356531 | WD repeat domain 66 |
| 252 | WNT6 | 2 | 219736286-219736388 | wingless-type MMTV integration |
| | | | | site family, member 6 |
| 253 | ZDHHC18 | 1 | 27160118-27160221 | zinc finger, DHHC-type |
| | | | | containing 18 |
| 254 | ZNF382 | 19 | 37096235-37096414 | zinc finger protein 382 |
| 255 | ZNF626 | 19 | 20844163-20844274 | zinc finger protein 626 |

For OPSCC validation, 62 candidates were chosen (Table 8). These were the among the top ranked MDMs in terms of AUC, fold-change, Δ methylation, and p-value. Methylation-specific PCR assays were developed for testing on the discovery tissue samples. Short amplicon primers (<150 bp) were designed to target the most discriminant CpGs with in a DMR and assay checked on controls to ensure that fully methylated fragments amplified robustly and in a linear fashion, and that unmethylated and/or unconverted fragments did not amplify.

TABLE 8
Representative data for validated oropharyngeal DMRs (OPX), including AUC
and fold-change, as compared to normal tissue and buffy coat controls.

| | | AUC | Fold-Change | P-value |
|---|---|---|---|---|
| DMR. | | OPX vs. Normal | OPX vs. Normal | OPX vs. Normal |
| No. | Gene Annotation | Tissue | Tissue | Tissue |
| 22 | ALX4 | 0.9183 | 6.986 | 8.31E−07 |
| 24 | C1orf114_B (C1orf114_6622) | 0.8642 | 13.12 | 8.74E−06 |
| 26 | CA8 | 0.9281 | 18.93 | 2.65E−05 |
| 30 | CCNA1 | 0.9771 | 9.228 | 3.76E−07 |
| 32 | CLSTN2 | 0.951 | 11.95 | 0.002349 |
| 33 | CR1 | 0.9248 | 16.16 | 0.0005182 |
| 35 | DAB1 | 0.9343 | 10.4 | 6.78E−05 |
| 37 | DOK1 | 0.8993 | 12.18 | 3.27E−05 |
| 42 | EMBP1 | 0.9183 | 23.01 | 6.53E−05 |
| 43 | EPDR1_0389 (region 1) | 0.9412 | 12.75 | 7.98E−06 |
| 45 | FLJ43390 | 0.9281 | 20.02 | 2.38E−05 |
| 46 | FMN2 | 0.8971 | 24.94 | 1.96E−06 |
| 51 | GDF6 | 0.9431 | 6.541 | 1.87E−05 |
| 52 | GFRA1 | 0.9346 | 10.16 | 4.29E−06 |
| 54 | HOXB3 | 0.8758 | 19.62 | 2.05E−05 |
| 57 | LDLRAD2 | 0.9052 | 12.27 | 2.67E−06 |
| 64 | LOC648809 | 0.915 | 12.68 | 7.78E−06 |
| 67 | MAPRE2 | 0.902 | 36.88 | 0.0004141 |
| 70 | MAX.chr1.241587339-241587784 | 0.9314 | 13.8 | 5.13E−05 |
| 71 | MAX.chr1.50798781-50799423 | 0.9118 | 13.12 | 1.21E−06 |
| 76 | MAX.chr13.28527984-28528214 | 0.8627 | 14.61 | 0.0001449 |
| 79 | MAX.chr16.3221176-3221223 | 0.9314 | 13.96 | 1.46E−06 |
| 82 | MAX.chr19.11805263-11805639 | 0.9444 | 14.01 | 6.11E−07 |
| 85 | MAX.chr19.22034646-22034887 | 0.9052 | 22.27 | 0.0004558 |
| 89 | MAX.chr19.30718373-30719719 | 1 | 28.8 | 1.21E−06 |
| 92 | MAX.chr2.173099712-173099791 | 0.9537 | 26.08 | 0.01809 |
| 93 | MAX.chr2.66808635-66808731 | 0.9346 | 8.158 | 6.91E−06 |
| 98 | MAX.chr6.38683091-38683226 | 0.9706 | 12.27 | 9.49E−06 |
| 103 | MAX.chr9.79638034-79638077 | 0.8979 | 13.32 | 6.34E−06 |
| 106 | MECOM | 0.915 | 14.77 | 1.16E−05 |
| 112 | ONECUT2 | 0.9526 | 11.25 | 7.71E−07 |
| 114 | PARP15 | 0.8954 | 26.59 | 8.73E−06 |
| 120 | SGIP1 | 0.8856 | 11.03 | 2.60E−05 |
| 121 | SIM2 | 0.8981 | 12.41 | 3.39E−07 |
| 125 | SORCS3_0952 (region 1) | 0.9412 | 10.55 | 1.51E−06 |
| 126 | ST6GALNAC5 | 0.9281 | 15.69 | 1.11E−05 |
| 127 | ST8SIA5 | 0.9444 | 19.4 | 7.50E−07 |
| 130 | TFAP2E | 0.9003 | 13.99 | 0.001418 |
| 131 | TLX2 | 0.9216 | 10.41 | 2.70E−07 |
| 132 | TLX3 | 0.9216 | 13.74 | 4.76E−07 |
| 136 | VSTM2B_6096 (region 1) | 0.9085 | 10.58 | 1.82E−06 |
| 138 | WDR17 | 0.9379 | 18.67 | 8.87E−05 |
| 139 | ZNF254 | 0.9379 | 27.77 | 0.0012 |
| 140 | ZNF43 | 0.9641 | 23.56 | 0.0004046 |
| 142 | ZNF491 | 0.9183 | 20.12 | 2.25E−05 |
| 149 | ZNF763 | 0.9314 | 28.38 | 0.0001029 |
| 150 | ZNF844 | 0.9248 | 18.19 | 9.24E−05 |

| | | AUC | Fold-Change | P-value |
|---|---|---|---|---|
| DMR. | | OPX vs. | OPX vs. | OPX vs. |
| No. | Gene Annotation | Buffy Coat | Buffy Coat | Buffy Coat |
| 168 | FAM19A2 | 0.9967 | 48.96 | 0.00726 |
| 175 | IFFO1 | 0.9967 | 78.83 | 0.002848 |
| 178 | ITGB4 | 1 | 127.4 | 0.0006248 |
| 186 | LOC100192379 | 0.9706 | 73.03 | 0.005885 |
| 190 | MAX.chr1.84326495-84326656 | 0.9869 | 43.82 | 0.001622 |
| 194 | MAX.chr16.85482306-85482494 | 0.9575 | 146 | 0.003815 |
| 203 | MAX.chr6.45631561-45631625 | 0.9739 | 65.16 | 0.0003116 |
| 204 | MAX.chr7.25892382-25892451 | 1 | 91.31 | 0.002958 |
| 217 | MT1IP | 0.9653 | 64.48 | 0.0003501 |
| 218 | NCOR2 | 0.9902 | 71.64 | 0.001616 |
| 223 | OLIG1_2354 (region 1) | 0.9673 | 53.7 | 0.006518 |
| 234 | RAD52 | 1 | 76.19 | 0.007544 |
| 241 | SHROOM1 | 1 | 74.69 | 1.85E−06 |
| 243 | SLC12A8 | 1 | 173.8 | 0.01137 |
| 248 | TBC1D10C | 1 | 67.28 | 0.0001266 |

The results were analyzed logistically to determine AUC and fold change. The analyses for the tissue and buffy coat controls were run separately. Results for the qMSP assay are provided in Table 9. One DMR, ZNF763, was 100% discriminant in separating cancer from benign tissue and 18 MDMs perfectly discriminated cancer from the buffy coat samples, an important characteristic for liquid biopsy applications.

TABLE 9
Representative qMPS data for validated oropharyngeal DMRs (OPX), including
AUC and fold-change, as compared to normal tissue and buffy coat controls.

| | | AUC | AUC | Fold-Change | Fold-Change |
|---|---|---|---|---|---|
| | | OPX vs. | OPX vs. | OPX vs. | OPX vs. |
| DMR. | | Normal | Normal | Normal | Normal |
| No. | Gene Annotation | Tissue | Buffy Coat | Tissue | Buffy Coat |
| 22 | ALX4 | 0.875 | 0.9485 | 18 | 41 |
| 24 | C1orf114_B (C1orf114_6622) | 0.9007 | 0.9926 | 14 | 179 |
| 26 | CA8 | 0.9669 | 1 | 16 | 36 |
| 30 | CCNA1 | 0.9191 | 0.9632 | 20 | 36 |
| 32 | CLSTN2 | 0.6985 | 0.7206 | 11 | 16 |
| 33 | CR1 | 0.5515 | 0.6838 | 1 | 2 |
| 35 | DAB1 | 0.7721 | 0.886 | 8 | 38 |
| 37 | DOK1 | 0.8824 | 0.9522 | 5 | 11 |
| 42 | EMBP1 (version 1) | 0.9412 | 1 | 10 | 20 |
| 42 | EMBP1 (version 2) | 0.9301 | 0.9375 | 35 | 52 |
| 43 | EPDR1_0389 (region 1) | 0.9265 | 0.9375 | 28 | 81 |
| 168 | FAM19A2 | 0.9191 | 0.9926 | 7 | 26 |
| 45 | FLJ43390 (version 1) | 0.9007 | 0.9522 | 9 | 19 |
| 45 | FLJ43390 (version 2) | 0.6912 | 0.8051 | 3 | 6 |
| 46 | FMN2 | 0.9412 | 0.9706 | 11 | 22 |
| 51 | GDF6 | 0.6618 | 0.7868 | 4 | 14 |
| 52 | GFRA1 | 0.489 | 0.6875 | 1 | 2 |
| 54 | HOXB3 | 0.9853 | 1 | 23 | 436 |
| 175 | IFFO1 | 0.8658 | 1 | 3 | 29 |
| 178 | ITGB4 | 0.8272 | 1 | 4 | 188 |
| 57 | LDLRAD2 | 0.9412 | 0.9743 | 12 | 27 |
| 186 | LOC100192379 | 0.8015 | 1 | 7 | 50 |
| 64 | LOC648809 | 0.9926 | 1 | 19 | 99 |
| 67 | MAPRE2 | 0.9007 | 0.9357 | 46 | 497 |
| 70 | MAX.chr1.241587339-241587784 | 0.8051 | 0.9191 | 4 | 9 |
| | (version 1) | | | | |
| 70 | MAX.chr1.241587339-241587784 | 0.9265 | 0.9596 | 16 | 16 |
| | (version 2) | | | | |
| 71 | MAX.chr1.50798781-50799423 | 0.9154 | 0.9779 | 21 | 46 |
| 190 | MAX.chr1.84326495-84326656 | 0.8529 | 1 | 7 | 148 |
| 76 | MAX.chr13.28527984-28528214 | 0.8364 | 0.8088 | 81 | 75 |
| 79 | MAX.chr16.3221176-3221223 | 0.989 | 1 | 25 | 139 |
| 194 | MAX.chr16.85482306-85482494 | 0.6544 | 1 | 2 | 37 |
| 82 | MAX.chr19.11805263-11805639 | 0.9669 | 0.9853 | 13 | 20 |
| 85 | MAX.chr19.22034646-22034887 | 0.7831 | 0.9449 | 9 | 26 |
| 89 | MAX.chr19.30718373-30719719 | 0.8493 | 0.9301 | 10 | 20 |
| | (version 1) | | | | |
| 89 | MAX.chr19.30718373-30719719 | 0.9412 | 1 | 5 | 13 |
| | (version 2) | | | | |
| 92 | MAX.chr2.173099712-173099791 | 0.9081 | 0.9007 | 13 | 5 |
| 93 | MAX.chr2.66808635-66808731 | 0.9044 | 0.9963 | 6 | 51 |
| 98 | MAX.chr6.38683091-38683226 | 0.8971 | 0.932 | 18 | 43 |
| 203 | MAX.chr6.45631561-45631625 | 0.8603 | 0.9963 | 5 | 22 |
| 204 | MAX.chr7.25892382-25892451 | 0.6912 | 0.9485 | 3 | 21 |
| 103 | MAX.chr9.79638034-79638077 | 0.8235 | 1 | 10 | 43 |
| 106 | MECOM | 0.9301 | 0.9522 | 32 | 439 |
| 217 | MT1IP | 0.9412 | 1 | 10 | 124 |
| 218 | NCOR2_1781 (region 1) | 0.8566 | 0.8971 | 16 | 37 |
| 223 | OLIG1 | 0.7757 | 0.9449 | 5 | 20 |
| 112 | ONECUT2 | 0.9669 | 1 | 14 | 47 |
| 114 | PARP15 | 0.9669 | 0.9926 | 111 | 241 |
| 234 | RAD52 | 0.5239 | 0.4614 | 3 | 1 |
| 120 | SGIP1 | 0.8824 | 0.9743 | 26 | 225 |
| 241 | SHROOM1 | 0.9118 | 1 | 5 | 65 |
| 121 | SIM2 | 0.9228 | 1 | 6 | 71 |
| 243 | SLC12A8 | 0.9081 | 1 | 9 | 56 |
| 125 | SORCS3 (region 1; SORCS3_0952) | 0.4669 | 0.6471 | 1 | 2 |
| 125 | SORCS3 (region 2; SORCS3_2558) | 0.9375 | 0.9963 | 17 | 47 |
| 126 | ST6GALNAC5 | 0.8603 | 0.9926 | 10 | 49 |
| 127 | ST8SIA5 | 0.9522 | 0.9596 | 123 | 287 |
| 248 | TBC1D10C | 0.5882 | 0.9816 | 2 | 55 |
| 130 | TFAP2E | 0.7978 | 0.8603 | 18 | 41 |
| 131 | TLX2 | 0.8382 | 0.8971 | 4 | 8 |
| 132 | TLX3 | 0.9963 | 1 | 22 | 163 |
| 136 | VSTM2B_6096 (region 1) | 0.8346 | 0.9191 | 13 | 59 |
| 138 | WDR17 | 0.9191 | 0.9265 | 80 | 104 |
| 139 | ZNF254 | 0.9228 | 0.9044 | 42 | 34 |
| 140 | ZNF43 | 0.9191 | 0.9816 | 42 | 342 |
| 142 | ZNF491 | 0.9412 | 0.9375 | 58 | 42 |
| 149 | ZNF763 | 1 | 0.9816 | 98 | 39 |
| 150 | ZNF844 | 0.9559 | 0.9632 | 30 | 40 |

Additionally, 39 of the 62 DMRs were utilized for further validation experiments (Table 10). These DMRs had tissue to tissue AUCs above 0.80 and/or tissue to buffy coat AUCs above 0.90.

TABLE 10
Validated DMRs, including AUC and fold-change, as
compared to normal tissue and buffy coat controls.

| | | AUC | AUC | Fold-Change | Fold-Change |
|---|---|---|---|---|---|
| | | OPX vs. | OPX vs. | OPX vs. | OPX vs. |
| DMR. | | Normal | Normal | Normal | Normal |
| No. | Gene Annotation | Tissue | Buffy Coat | Tissue | Buffy Coat |
| 24 | C1orf114_B (C1orf114_6622) | 0.9007 | 0.9926 | 14 | 179 |
| 26 | CA8 | 0.9669 | 1 | 16 | 36 |
| 30 | CCNA1 | 0.9191 | 0.9632 | 20 | 36 |
| 42 | EMBP1 (version 1) | 0.9412 | 1 | 10 | 20 |
| 42 | EMBP1 (version 2) | 0.9301 | 0.9375 | 35 | 52 |
| 43 | EPDR1_0389 (region 1) | 0.9265 | 0.9375 | 28 | 81 |
| 168 | FAM19A2 | 0.9191 | 0.9926 | 7 | 26 |
| 46 | FMN2 | 0.9412 | 0.9706 | 11 | 22 |
| 54 | HOXB3 | 0.9853 | 1 | 23 | 436 |
| 175 | IFFO1 | 0.8658 | 1 | 3 | 29 |
| 178 | ITGB4 | 0.8272 | 1 | 4 | 188 |
| 57 | LDLRAD2 | 0.9412 | 0.9743 | 12 | 27 |
| 186 | LOC100192379 | 0.8015 | 1 | 7 | 50 |
| 64 | LOC648809 | 0.9926 | 1 | 19 | 99 |
| 67 | MAPRE2 | 0.9007 | 0.9357 | 46 | 497 |
| 71 | MAX.chr1.50798781-50799423 | 0.9154 | 0.9779 | 21 | 46 |
| 190 | MAX.chr1.84326495-84326656 | 0.8529 | 1 | 7 | 148 |
| 79 | MAX.chr16.3221176-3221223 | 0.989 | 1 | 25 | 139 |
| 82 | MAX.chr19.11805263-11805639 | 0.9669 | 0.9853 | 13 | 20 |
| 93 | MAX.chr2.66808635-66808731 | 0.9044 | 0.9963 | 6 | 51 |
| 98 | MAX.chr6.38683091-38683226 | 0.8971 | 0.932 | 18 | 43 |
| 203 | MAX.chr6.45631561-45631625 | 0.8603 | 0.9963 | 5 | 22 |
| 103 | MAX.chr9.79638034-79638077 | 0.8235 | 1 | 10 | 43 |
| 106 | MECOM | 0.9301 | 0.9522 | 32 | 439 |
| 217 | MT1IP | 0.9412 | 1 | 10 | 124 |
| 112 | ONECUT2 | 0.9669 | 1 | 14 | 47 |
| 114 | PARP15 | 0.9669 | 0.9926 | 111 | 241 |
| 241 | SHROOM1 | 0.9118 | 1 | 5 | 65 |
| 121 | SIM2 | 0.9228 | 1 | 6 | 71 |
| 243 | SLC12A8 | 0.9081 | 1 | 9 | 56 |
| 125 | SORCS3 (region 1; SORCS3_0952) | 0.4669 | 0.6471 | 1 | 2 |
| 125 | SORCS3 (region 2; SORCS3_2558) | 0.9375 | 0.9963 | 17 | 47 |
| 126 | ST6GALNAC5 | 0.8603 | 0.9926 | 10 | 49 |
| 127 | ST8SIA5 | 0.9522 | 0.9596 | 123 | 287 |
| 248 | TBC1D10C | 0.5882 | 0.9816 | 2 | 55 |
| 132 | TLX3 | 0.9963 | 1 | 22 | 163 |
| 139 | ZNF254 | 0.9228 | 0.9044 | 42 | 34 |
| 142 | ZNF491 | 0.9412 | 0.9375 | 58 | 42 |
| 149 | ZNF763 | 1 | 0.9816 | 98 | 39 |
| 150 | ZNF844 | 0.9559 | 0.9632 | 30 | 40 |

DNA from 10 normal saliva cell pellets were also tested with 7 DMRs to confirm the assays as currently configured are suitable for this sample type (Table 11). Note that 3 of the 7 DMRs are virtually silent while the others demonstrate differing degrees of hypermethylation. These results are in line with what would be predicted from the RRBS data.

TABLE 11
DMRs identified in saliva samples as being hypermethylated.

| | | | | MAX chr16 | | |
|---|---|---|---|---|---|---|
| HOXB3 | LOC648809 | TLX3 | ZNF763 | 3221176-3221223 | TBC1D10C | SHROOM1 |
| 5.61 | 0.00 | 0.01 | 0.00 | 3.46 | 56.61 | 22.75 |
| 2.26 | 0.00 | 0.01 | 0.00 | 1.39 | 17.49 | 2.27 |
| 4.39 | 0.00 | 0.28 | 0.00 | 2.67 | 39.26 | 10.44 |
| 1.80 | 0.00 | 0.01 | 0.00 | 2.18 | 20.04 | 5.08 |
| 5.86 | 0.00 | 0.46 | 0.00 | 1.94 | 50.35 | 9.08 |
| 2.09 | 0.00 | 0.01 | 0.00 | 1.82 | 6.97 | 8.34 |
| 4.12 | 0.00 | 0.29 | 0.00 | 2.14 | 22.19 | 10.13 |
| 7.37 | 0.33 | 0.01 | 0.01 | 1.98 | 20.56 | 14.77 |
| 1.72 | 0.00 | 0.01 | 0.00 | 1.15 | 22.82 | 3.64 |
| 6.40 | 0.00 | 0.46 | 0.00 | 3.33 | 42.27 | 17.74 |

Taken together, the DMRs developed for the detection of oropharyngeal cancers demonstrated excellent performance through validation with respect to both normal tissue and normal WBCs (buffy coat) control samples. The DMR markers disclosed in the present disclosure for oropharyngeal cancer, as well as the assays constructed to assess them, are uniquely suitable for detecting these cancers in a non-invasive, clinical setting.

In accordance with the above data, the following materials and method were used. The primer sequences used in the assays described above are listed in Table 12. Note that for 5 DMRs, two different versions of primer pairs were used due to the number of discriminate CpGs. These DMRs include EMBP1, FLJ43390, MAX_chr1_241587339_241587784, MAX_chr19_30718373_30719719, and SORCS3.

TABLE 12
DMRs identifying oropharyngeal cancers and their corresponding primer
sequences.

| | | Forward | SEQ | Reverse | SEQ |
|---|---|---|---|---|---|
| DMR | | Primer 5′-3′ | ID | Primer 5′-3′ | ID |
| No. | Gene Annotation | Sequence (hg19) | NO: | Sequence (hg19) | NO: |
| 22 | ALX4 | CGGTACGTAGTAGGA | 43 | ACAACTCCACATTTA | 44 |
| | | AAATAGATTAAAACG | | CTTTCACGCCAACG | |
| | | T | | | |
| 24 | Clorf114_B | TTGCGTAGATTTTTGT | 45 | GTACCTCTACGTCCTC | 46 |
| | (Clorf114_6622) | CGTTTTCGT | | CATCCGAA | |
| 26 | CA8 | TGGAATATTCGGTTC | 47 | AACGAAAAAACGATT | 48 |
| | | GAACGTACGT | | ACTCTACGCA | |
| 30 | CCNA1 | GTTGGGTTTTTAGGG | 49 | AACGAAAACTAAACG | 50 |
| | | GTTTGAACGT | | CTAAAATAAAAACGA | |
| 32 | CLSTN2 | GGTTTCGATTTTTAAT | 51 | CGCTACCCAAAAATT | 52 |
| | | TTTTTTTAGTCGT | | CCCTTCCCG | |
| 33 | CR1 | GTTGTGGTGTTGTTTG | 53 | TCGACCCCCTCTCTCC | 54 |
| | | CGTTGTCGG | | CGAC | |
| 35 | DAB1 | GGATGTTTATTTTTAG | 55 | ACAACTCAACCCAAA | 56 |
| | | CGGTTTCGT | | ACCGCAAACG | |
| 37 | DOK1 | AGTTCGGCGTTATTG | 57 | CTACAAACCCTCCCC | 58 |
| | | TTTTTCGT | | ACGCT | |
| 42 | EMBP1 (version 1) | CGTATATTCGGGAAC | 59 | GCGAACCTACGCGCG | 60 |
| | | GTTAGTAGCGG | | AAAAAACG | |
| 42 | EMBP1 (version 2) | GTAGTTAGTGTCGCG | 61 | ACATTCCCCAACCCT | 62 |
| | | GGTAGGACGT | | ATCGAACGTT | |
| 43 | EPDR1_0389 (region 1) | TCGGTATAGTGCGGA | 63 | ACTTCCAAAACCGAA | 64 |
| | | AAGAGTCGGC | | CGAAATCGAA | |
| 168 | FAM19A2 | CGGTTCGTAAAAGTT | 65 | AAAACATTCTAAAAA | 66 |
| | | GTAGGAGTATCGT | | AAACTTCGCC | |
| 45 | FLJ43390 (version 1) | TTTCGAGTTGTGGAG | 67 | AAAAAACGAAAACCT | 68 |
| | | GGGCGTTATC | | AAACCCGAC | |
| 45 | FLJ43390 (version 2) | GGGGCGTTATCGTTT | 69 | ATTCAATAATCCCGA | 70 |
| | | TTTAGTAACGT | | AATCCCCGAA | |
| 46 | FMN2 | TAGGGAGTTTTTCGG | 71 | AACACTCGAATACCG | 72 |
| | | GAGAGTTAGGCGG | | CGCGA | |
| 51 | GDF6 | TTCGGTTGATTATTTT | 73 | GCGCTAACTATTCTC | 74 |
| | | ATTTAGCGT | | GATTACGCC | |
| 52 | GFRA1 | TAGTTGTTCGGGGAT | 75 | CGAAATACCGAAATA | 76 |
| | | CGTTGTACGT | | AACCAAACCGA | |
| 54 | HOXB3 | TTTTTCGGGATTAGA | 77 | CAAAAATATACCGAA | 78 |
| | | GAGAGAGCGA | | AAATAACGTCGTT | |
| 175 | IFFO1 | ATGATAGGTTTTAAT | 79 | CCGAACGCGTCGCTA | 80 |
| | | TGGAGGTCGG | | CTTTCGAC | |
| 178 | ITGB4 | TTTTAATATATATTTC | 81 | CCTTCGCGATCCCTTA | 82 |
| | | GGGATTTCGG | | CTACGAA | |
| 57 | LDLRAD2 | TAGGTTGTAGATTAG | 83 | TTAATCTCCCGCTAC | 84 |
| | | GAAGAAGCGG | | AAATCCCGCT | |
| 186 | LOC100192379 | CGGGGGAGGTAAAGT | 85 | AACCCGAAAATATAT | 86 |
| | | ACGGATTTCGG | | AATAAAACGCCGAA | |
| 64 | LOC648809 | CGGGGGAGGTAAAGT | 87 | TCCCGAATTCGTTAT | 88 |
| | | ACGGATTTCGG | | AATCCCCGCC | |
| 67 | MAPRE2 | GTTTTCGGGATTTAG | 89 | CAACCTAAACGCAAA | 90 |
| | | GAGTCGG | | ATACCCCGAA | |
| 70 | MAX.chr1.241587339- | GGTTTGGGATTAGGA | 91 | AATACCGAAACGACA | 92 |
| | 241587784 (version 1) | GTTGGGGTCG | | AAAAACCGAA | |
| 70 | MAX.chr1.241587339- | TTAGGGGATTTTAGC | 93 | CTAAACGAACAAACG | 94 |
| | 241587784 (version 2) | GGGAGTCGG | | AAAAAAACGA | |
| 71 | MAX.chr1.50798781- | CGGGAAAGGGGTTCG | 95 | CAAAACGACTTCCGA | 96 |
| | 50799423 | GATTTTATACGT | | AATATAACGAA | |
| 190 | MAX.chr1.84326495- | TTTGGTGTCGCGTTTT | 97 | AACTATAAATCCCCC | 98 |
| | 84326656 | TATTAGTTC | | AATACCCGCA | |
| 76 | MAX.chr13.28527984- | CGGGGTATTTTTTAGT | 99 | CCCTACTTCTTAATAT | 100 |
| | 28528214 | TTTTTTTGC | | CCCGACGTC | |
| 79 | MAX.chr16.3221176- | CGTGGTCGGGAGTTT | 101 | ACGCAACCGATAATT | 102 |
| | 3221223 | ACGATCGC | | AATATCCGCT | |
| 194 | MAX.chr16.85482306- | ATTCGGGGTAGGAAA | 103 | AATCTTTAACCCCTCC | 104 |
| | 85482494 | TTATTAACGT | | CGCCGAA | |
| 82 | MAX.chr19.11805263- | GGTTCGTAGTCGTCG | 105 | ACAACTCCACATTTA | 106 |
| | 11805639 | CGAAGGGAC | | CTTTCACGCCAACG | |
| 85 | MAX.chr19.22034646- | CGATAAAGTTATCGG | 107 | CGAAACAACGCAACC | 108 |
| | 22034887 | GGATTTTCGA | | GCT | |
| 89 | MAX.chr19.30718373- | TTTCGTTTTTATTTTA | 109 | AACGAAAAAACGATT | 110 |
| | 30719719 (version 1) | GTAGAAAATACGG | | ACTCTACGCA | |
| 89 | MAX.chr19.30718373- | TGATGGTGAAATAGT | 111 | AACGAAAACTAAACG | 112 |
| | 30719719 (version 2) | AAATTTACGTACGGA | | CTAAAATAAAAACGA | |
| 92 | MAX.chr2.173099712- | TTAACGGTAGTATCG | 113 | CGCTACCCAAAAATT | 114 |
| | 173099791 | GTAGTATCGA | | CCCTTCCCG | |
| 93 | MAX.chr2.66808635- | GGTTATTTGTTGTTCG | 115 | TCGACCCCCTCTCTCC | 116 |
| | 66808731 | TTTTGCGGGGAC | | CGAC | |
| 98 | MAX.chr6.38683091- | TTTCGATTATTTAGTT | 117 | ACAACTCAACCCAAA | 118 |
| | 38683226 | TATTGAGGTCGG | | ACCGCAAACG | |
| 203 | MAX.chr6.45631561- | TCGGGTTTGTTTTACG | 119 | CTACAAACCCTCCCC | 120 |
| | 45631625 | TTGAAACGT | | ACGCT | |
| 204 | MAX.chr7.25892382- | CGGGAAAACGTCGTA | 121 | GCGAACCTACGCGCG | 122 |
| | 25892451 | GTCGGCGT | | AAAAAACG | |
| 103 | MAX.chr9.79638034- | GAAAGTCGGCGGTTA | 123 | ACATTCCCCAACCCT | 124 |
| | 79638077 | AGTTGTTCGA | | ATCGAACGTT | |
| 106 | MECOM | CGTTCGTTTTTAGCGG | 125 | ACTTCCAAAACCGAA | 126 |
| | | AGGTAACGC | | CGAAATCGAA | |
| 217 | MT1IP | AGAGTATTTTTTTTAT | 127 | AAAACATTCTAAAAA | 128 |
| | | TTATTTTTACGCGG | | AAACTTCGCC | |
| 218 | NCOR2_1781 (region 1) | CGAAATTTGGTTGGT | 129 | AAAAAACGAAAACCT | 130 |
| | | TTGTTGGGGTC | | AAACCCGAC | |
| 223 | OLIG1_2354 (region 1) | TTTAAGTCGGGTTTA | 131 | ATTCAATAATCCCGA | 132 |
| | | AAGGGGTAGGGCG | | AATCCCCGAA | |
| 112 | ONECUT2 | CGATTTTTCGGGTTTT | 133 | AACACTCGAATACCG | 134 |
| | | TTTCGTATCGT | | CGCGA | |
| 114 | PARP15 | GAAGAGCGTCGGGAG | 135 | GCGCTAACTATTCTC | 136 |
| | | GTTTTTCGC | | GATTACGCC | |
| 234 | RAD52 | CGTCGCGTATATTTA | 137 | CGAAATACCGAAATA | 138 |
| | | GTCGTTATCGG | | AACCAAACCGA | |
| 120 | SGIP1 | TCGTTTGTAGTTCGG | 139 | CAAAAATATACCGAA | 140 |
| | | ACGGCGA | | AAATAACGTCGTT | |
| 241 | SHROOM1 | GCGGTTGTAGTTCGT | 141 | CCGAACGCGTCGCTA | 142 |
| | | AGTGGGTCGT | | CTTTCGAC | |
| 121 | SIM2 | TTGGGTTCGGTTAAA | 143 | CCTTCGCGATCCCTTA | 144 |
| | | GTCGT | | CTACGAA | |
| 243 | SLC12A8 | TTATTTTTGGATTAGC | 145 | TTAATCTCCCGCTAC | 146 |
| | | GATCGACGA | | AAATCCCGCT | |
| 125 | SORCS3 (region 1; | TAAGGATGTATCGAG | 147 | AACCCGAAAATATAT | 148 |
| | SORCS3_0952) | AGGTGGCGTCG | | AATAAAACGCCGAA | |
| 125 | SORCS3 (region 2; | CGGAATGTCGGAGTA | 149 | TCCCGAATTCGTTAT | 150 |
| | SORCS3_2558) | GGTTAGATCGG | | AATCCCCGCC | |
| 126 | ST6GALNAC5 | TTTTTATCGAGAGATT | 151 | CAACCTAAACGCAAA | 152 |
| | | TGGGCGGCG | | ATACCCCGAA | |
| 127 | ST8SIA5 | GGCGGGTTACGTTTG | 153 | AATACCGAAACGACA | 154 |
| | | ATTTTCGG | | AAAAACCGAA | |
| 248 | TBC1D10C | AATTGTTTTTTATAGG | 155 | CTAAACGAACAAACG | 156 |
| | | TGTATAGCGT | | AAAAAAACGA | |
| 130 | TFAP2E | ATTGTAGGTAAGCGT | 157 | CAAAACGACTTCCGA | 158 |
| | | TCGGGAGGGGC | | AATATAACGAA | |
| 131 | TLX2 | GGGTTTGTAGTTTATC | 159 | AACTATAAATCCCCC | 160 |
| | | GGGCGTCGT | | AATACCCGCA | |
| 132 | TLX3 | GGGGTCGTTGGAGGA | 161 | CCCTACTTCTTAATAT | 162 |
| | | AGGGTAGTC | | CCCGACGTC | |
| 136 | VSTM2B_6096 | ATGTCGAGTACGTTA | 163 | ACGCAACCGATAATT | 164 |
| | (region 1) | GAGTAGGCGG | | AATATCCGCT | |
| 138 | WDR17 | TGCGAGGCGAGTATT | 165 | AATCTTTAACCCCTCC | 166 |
| | | ATTAACGCGC | | CGCCGAA | |
| 139 | ZNF254 | AGATTTGGGATTTCG | 167 | ACAACTCCACATTTA | 168 |
| | | GCGATAGCGA | | CTTTCACGCCAACG | |
| 140 | ZNF43 | TTGGAGTTTTGCGTTT | 169 | CGAAACAACGCAACC | 170 |
| | | CGTTTTCGT | | GCT | |
| 142 | ZNF491 | GAGGTTTTAGGTGTT | 171 | AACGAAAAAACGATT | 172 |
| | | TTCGTTACGG | | ACTCTACGCA | |
| 149 | ZNF763 | GGGTATAGCGTAGGA | 173 | AACGAAAACTAAACG | 174 |
| | | GATAGAGCGA | | CTAAAATAAAAACGA | |
| 150 | ZNF844 | CGGTTTTTTTTAGAGT | 175 | CGCTACCCAAAAATT | 176 |
| | | AGGAAAAACGG | | CCCTTCCCG | |

Samples. FFPE tissues were obtained from 18 HPV+ oropharyngeal squamous cell carcinomas (OPSCC), (9 stage I, 7 stage II, and 2 stage III) and 18 cancer free patients (tonsil). Samples were matched by age and gender. All tissues were provided by the Mayo Clinic Tissue Registry. Eighteen normal buffy coat samples from the NOMAD collection were also included in the study. Genomic DNA was purified using the QIAamp FFPE Mini kit (FFPE) and QIAamp DNA Blood Mini kit (buffy coat) (Qiagen, Valencia CA). DNA was re-purified with AMPure XP beads (Beckman-Coulter, Brea CA) and quantified by PicoGreen (Thermo-Fisher, Waltham MA). DNA integrity was assessed using qPCR.

Sequencing. RRBS sequencing libraries were prepared using the Ovation RRBS Methyl-Seq library preparation kit with modifications (Tecan Genomics, Redwood City CA). Briefly, samples were digested with Msp1, ligated to indexed flow cell adapters, bisulfite converted (twice), amplified, combined in a 4-plex format, and sequenced by the Mayo Genomics Facility on the Illumina HiSeq 4000 instrument (Illumina, San Diego CA). Reads were processed by Illumina pipeline modules for image analysis and base calling. Secondary analysis was performed using SAAP-RRBS, a Mayo developed bioinformatics suite. Briefly, reads were cleaned-up using Trim-Galore and aligned to the GRCh37/hg19 reference genome build with BSMAP. Methylation ratios were determined by calculating C/(C+T) or conversely, G/(G+A) for reads mapping to reverse strand, for CpGs with coverage ≥10× and base quality score ≥20.

Biomarker Selection. A proprietary identification pipeline and regression package was used to derive regions of significant differential methylation (DMRs) The difference in average methylation percentage was compared between cases, tissue controls and buffy coat controls; a tiled reading frame within 100 base pairs of each mapped CpG was used to identify DMRs where control methylation was <5%, although this cut-off value was varied contingent on the stringency required. DMRs were only analyzed if the total depth of coverage was 10 reads per subject on average and the variance across subgroups was >0.

Following regression, DMRs were ranked by p-value, area under the receiver operating characteristic curve (AUC) and fold-change difference between cases and controls. No adjustments for false discovery were made during this phase as independent validation was planned a priori.

Specifically, individual CpGs within a DMR were ranked by hypermethylation ratio, namely the number of methylated cytosines at a given locus over the total cytosine count at that site. For cases, the ratios were required to be ≥0.20 (20%); for tissue controls, ≤0.05 (5%); for buffy coat controls, ≤0.01 (1%). DMRs ranged from 60-200 bp and included a minimum cut-off of 5 CpGs per region. DMRs with excessively high CpG density (>30%) were excluded to avoid GC-related amplification problems in the validation phase. For each candidate region, a 2-D methylation intensity heatmap was created which plotted individual CpGs within a region against case-control grouped samples. The methylated CpG patterns for OPSCC vs the respective benign controls and/or no-cancer buffy coat were analyzed. Final selections required coordinated and contiguous hypermethylation (in cases) of individual CpGs across the DMR sequence on a per sample level. Conversely, control samples had to have at least 10-fold less methylation than cases and the CpG pattern had to be empirically discordant.

Biomarker Validation. A subset of DMRs was chosen for further development. The criteria were primarily the logistic-derived area under the ROC curve metric which provides a performance assessment of the discriminant potential of the region. An AUC of 0.85 was chosen as the cut-off for the tissue-to tissue comparisons, and 0.95 for the tissue to buffy coat comparisons. In addition, the methylation fold-change ratio (average cancer hypermethylation ratio/average control hypermethylation ratio) was calculated and a lower limit of 10 was employed for tissue vs tissue comparisons and 20 for the tissue vs buffy coat comparisons. P-values were required to be less than 0.01. DMRs had to be concordantly methylated in cancers and discordant (or unmethylated) in controls. Case-control comparisons included the following: OPSCC vs tonsil tissue controls; and OPSCC vs normal buffy coat.

Quantitative methylation specific PCR (qMSP) primers were designed for candidate genomic hg19 regions using MethPrimer (Li L C and Dahiya R. MethPrimer: designing primers for methylation PCRs. Bioinformatics 2002 November; 18 (11): 1427-31 PMID: 12424112) and QC checked on 20 ng (6250 equivalents) of positive and negative genomic methylation controls. Multiple annealing temperatures were assessed for optimal discrimination. Validation was performed by qMSP on the sequenced DNA samples. This was done to verify that the DMRs contained CpGs that were truly discriminant by testing with an independent, non-NGS targeted PCR platform.

DNA purification was performed as previously described. The EZ-96 DNA Methylation kit (Zymo Research, Irvine CA) was used for the bisulfite conversion step. 10 ng of converted DNA (per marker) was amplified using SYBR Green detection on Roche 480 LightCyclers (Roche, Basel Switzerland). Serially diluted universal methylated genomic DNA (Zymo Research) was used as a quantitation standard. A CpG agnostic ACTB (β-actin) assay was used as an input reference and normalization control. Results were expressed as methylated copies (specific marker)/copies of ACTB.

Statistics. Results were analyzed logistically for individual MDMs (methylated DNA marker) performance.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

The present disclosure has multiple aspects, illustrated by the following non-limiting examples.

What is claimed is:

1. A method of characterizing a biological sample, the method comprising:
determining a methylation profile in at least one differentially methylated region (DMR) of a DNA sample obtained from a subject having or suspected of having an oropharyngeal cancer by treating the sample with a reagent that modifies DNA in a methylation-specific manner.

2. The method of claim 1, wherein the methylation profile in the at least one DMR indicates the subject has or is suspected of having HPV⁺ oropharyngeal squamous cell cancer (HPV⁺ OPSCC).

3. The method of claim 1 or claim 2, wherein the at least one DMR is from a gene selected from ABCB1, ARHGAP12, ASCL1, Clorf114, EMX1, GRIN2D, LOC645323, MAX.chr6.58147682-58147771, MAX.chr9.36739811-36739868, NEUROG3, NID2, TBX15, TMEM200C, TSPYL5, TTYH1, VWC2, ZNF610, ZNF69, ZNF773, ZNF781, ALX4, ATP10A, C1QL3, CA8, CACNA1A, CACNG8, CALCA, CCNA1, CLIC6, CLSTN2, CR1, CTNND2, DAB1, DGKG, DOK1, DOK6, DPP4, DUXA, ELMO1, EMBP1, EPDR1, FGF12, FLJ43390, FMN2, FOXB2, FOXD4, FREM3, GALR1, GDF6, GFRA1, GRIK3, HOXB3, HOXB4, HPSE2, LDLRAD2, LHX2, LOC100131366, LOC345643, LOC386758, LOC648809, LOC728392, MAML3, MAPRE2, MAX.chr1.226288154-226288189, MAX.chr1.2375078-2375126, MAX.chr1.241587339-241587784, MAX.chr1.50798781-50799423, MAX.chr10.22765150-22765477, MAX.chr10.23462342-23462436, MAX.chr11.14926602-14927044, MAX.chr11.58903531-58903592, MAX.chr13.28527984-28528214, MAX.chr13.29106641-29107037, MAX.chr14.100784488-100784782, MAX.chr16.3221176-3221223, MAX.chr16.3222040-3222098, MAX.chr16.71460171-71460282, MAX.chr19.11805263-11805639, MAX.chr19.16394457-16394646, MAX.chr19.21657626-21657769, MAX.chr19.22034646-22034887, MAX.chr19.23299989-23300156, MAX.chr19.30713427-30713588, MAX.chr19.30716926-30717074, MAX.chr19.30718373-30719719, MAX.chr2.118981724-118982174, MAX.chr2.127783107-127783403, MAX.chr2.173099712-173099791, MAX.chr2.66808635-66808731, MAX.chr22.50064113-50064259, MAX.chr3.137489884-137490061, MAX.chr5.138923141-138923219, MAX.chr5.42995180-42995535, MAX.chr6.38683091-38683226, MAX.chr7.121952014-121952084, MAX.chr7.155166980-155167310, MAX.chr8.99986792-99986864, MAX.chr9.79627078-79627116, MAX.chr9.79638034-79638077, MAX.chr9.98789824-98789847, MDFI, MECOM, MED12L, MIR129-2, MIR196A1, NELL1, NPY, ONECUT2, OPCML, PARP15, PDGFD, PEX5L, PRR15, SEMA6A, SFMBT2, SGIP1, SIM2, SLC35F3, SLCO4C1, SORCS3, ST6GALNAC5, ST8SIA5, SV2C, TACC2, TFAP2E, TLX2, TLX3, TRH, TRIM58, VAV3, VSTM2B, WDR17, ZNF254, ZNF43, ZNF486, ZNF491, ZNF518B, ZNF542, ZNF625, ZNF665, ZNF671, ZNF763, ZNF844, AGRN, ANKRD35, ARHGAP27, ARHGAP30, BCL2L11, BIN2, C10orf114, C4orf31, C6orf132, C6orf186, CCDC88B, CRHBP, DAPK1, DNMT3A, DPP10, FAM19A2, FLJ45983, FOSL1, FOXB1, GREM1, HMHA1, HOXA9, IFFO1, INPP4B, ITGB2, ITGB4, ITPKB, KCNIP2, KLHDC7B, LAT, LHX6, LIMK1, LOC100128239, LOC100192379, LOC646278, MAP2K2, MAX.chr1.210426156-210426257, MAX.chr1.84326495-84326656, MAX.chr10.119312785-119312882, MAX.chr15.67326025-67326060, MAX.chr16.54316401-54316453, MAX.chr16.85482306-85482494, MAX.chr17.74994454-74994572, MAX.chr17.76339840-76339972, MAX.chr2.7571082-7571136, MAX.chr21.45577347-45577679, MAX.chr3.14852538-14852568, MAX.chr3.187676564-187676668, MAX.chr4.174430662-174430793, MAX.chr5.177411809-177411836, MAX.chr6.45631561-45631625, MAX.chr7.25892382-25892451, MAX.chr7.402563-402641, MAX.chr7.64349554-64349606, MAX.chr8.142046239-142046398, MAX.chr8.145900842-145901246, MAX.chr9.126101804-126101848, MAX.chr9.126978999-126979182, MAX.chr9.36458633-36458725, MAX.chr9.87905315-87905326, MBP, MENG, MT1A, MT1IP, NCOR2, NFATC1, NKX3-2, NRN1, OLIG1, PALLD, PAPLN, PDLIM2, PKN1, PRDM14, PRKG1, PRMT7, PTGER2, PTK2B, RAD52, RBM38, RHOF, RNF220, RTN4RL1, RXRA, SDCCAG8, SHROOM1, SKI, SLC12A8, SLC25A47, SPEG, SUCLG2, TBC1D10C, TMEM132E, VIPR2, WDR66, WNT6, ZDHHC18, ZNF382, and ZNF626.

4. The method of claim 1 or claim 2, wherein the at least one DMR is from a gene selected from ABCB1, ARHGAP12, ASCL1, Clorf114, EMX1, GRIN2D, LOC645323, MAX.chr6.58147682-58147771, MAX.chr9.36739811-36739868, NEUROG3, NID2, TBX15, TMEM200C, TSPYL5, TTYH1, VWC2, ZNF610, ZNF69, ZNF773, and ZNF781.

5. The method of claim 1 or claim 2, wherein the at least one DMR is from a gene selected from ALX4, ATP10A, Clorf114, C1QL3, CA8, CACNA1A, CACNG8, CALCA, CCNA1, CLIC6, CLSTN2, CR1, CTNND2, DAB1, DGKG, DOK1, DOK6, DPP4, DUXA, ELMO1, EMBP1, EPDR1, FGF12, FLJ43390, FMN2, FOXB2, FOXD4, FREM3, GALR1, GDF6, GFRA1, GRIK3, HOXB3, HOXB4, HPSE2, LDLRAD2, LHX2, LOC100131366, LOC345643, LOC386758, LOC645323, LOC648809, LOC728392, MAML3, MAPRE2, MAX.chr1.226288154-226288189, MAX.chr1.2375078-2375126, MAX.chr1.241587339-241587784, MAX.chr1.50798781-50799423, MAX.chr10.22765150-22765477, MAX.chr10.23462342-23462436, MAX.chr11.14926602-14927044, MAX.chr11.58903531-58903592, MAX.chr13.28527984-28528214, MAX.chr13.29106641-29107037, MAX.chr14.100784488-100784782, MAX.chr16.3221176-3221223, MAX.chr16.3222040-3222098, MAX.chr16.71460171-71460282, MAX.chr19.11805263-11805639, MAX.chr19.16394457-16394646, MAX.chr19.21657626-21657769, MAX.chr19.22034646-22034887, MAX.chr19.23299989-23300156, MAX.chr19.30713427-30713588, MAX.chr19.30716926-30717074, MAX.chr19.30718373-30719719, MAX.chr2.118981724-118982174, MAX.chr2.127783107-127783403, MAX.chr2.173099712-173099791, MAX.chr2.66808635-66808731, MAX.chr22.50064113-50064259, MAX.chr3.137489884-137490061, MAX.chr5.138923141-138923219, MAX.chr5.42995180-42995535, MAX.chr6.38683091-38683226, MAX.chr7.121952014-121952084, MAX.chr7.155166980-155167310, MAX.chr8.99986792-99986864, MAX.chr9.79627078-79627116, MAX.chr9.79638034-79638077, MAX.chr9.98789824-98789847, MDFI, MECOM, MED12L, MIR129-2, MIR196A1, NELL1, NPY, ONECUT2, OPCML, PARP15, PDGFD, PEX5L, PRR15, SEMA6A, SFMBT2, SGIP1, SIM2, SLC35F3, SLCO4C1, SORCS3, ST6GALNAC5, ST8SIA5, SV2C, TACC2, TFAP2E, TLX2, TLX3, TRH, TRIM58, VAV3, VSTM2B, WDR17, ZNF254, ZNF43, ZNF486, ZNF491, ZNF518B, ZNF542, ZNF625, ZNF665, ZNF671, ZNF763, and ZNF844.

6. The method of claim 1 or claim 2, wherein the at least one DMR is from a gene selected from AGRN, ANKRD35, ARHGAP27, ARHGAP30, BCL2L11, BIN2, C10orf114, C4orf31, C6orf132, C6orf186, CCDC88B, CRHBP, DAPK1, DNMT3A, DPP10, ELMO1, EPDR1, FAM19A2, FLJ45983, FOSL1, FOXB1, GREM1, HMHA1, HOXA9, IFFO1, INPP4B, ITGB2, ITGB4, ITPKB, KCNIP2, KLHDC7B, LAT, LHX6, LIMK1, LOC100128239, LOC100192379, LOC646278, MAP2K2, MAX.chr1.210426156-210426257, MAX.chr1.84326495-84326656, MAX.chr10.119312785-119312882, MAX.chr15.67326025-67326060, MAX.chr16.54316401-54316453, MAX.chr16.85482306-85482494, MAX.chr17.74994454-74994572, MAX.chr17.76339840-76339972, MAX.chr2.7571082-7571136, MAX.chr21.45577347-45577679, MAX.chr3.14852538-14852568, MAX.chr3.187676564-187676668, MAX.chr4.174430662-174430793, MAX.chr5.177411809-177411836, MAX.chr6.45631561-45631625, MAX.chr7.25892382-25892451, MAX.chr7.402563-402641, MAX.chr7.64349554-64349606, MAX.chr8.142046239-142046398, MAX.chr8.145900842-145901246, MAX.chr9.126101804-126101848, MAX.chr9.126978999-126979182, MAX.chr9.36458633-36458725, MAX.chr9.87905315-87905326, MBP, MENG, MT1A, MT1IP, NCOR2, NFATC1, NKX3-2, NRN1, OLIG1, PALLD, PAPLN, PDLIM2, PKN1, PRDM14, PRKG1, PRMT7, PTGER2, PTK2B, RAD52, RBM38, RHOF, RNF220, RTN4RL1, RXRA, SDCCAG8, SHROOM1, SKI, SLC12A8, SLC25A47, SPEG, SUCLG2, TBC1D10C, TMEM132E, VIPR2, WDR66, WNT6, ZDHHC18, ZNF382, and ZNF626.

7. The method of claim 1 or claim 2, wherein the at least one DMR is from a gene selected from ALX4, Clorf114, CA8, CCNA1, CLSTN2, CR1, DAB1, DOK1, EMBP1, EPDR1, FLJ43390, FMN2, GDF6, GFRA1, HOXB3, LDLRAD2, LOC648809, MAPRE2, MAX.chr1.241587339-241587784, MAX.chr1.50798781-50799423, MAX.chr13.28527984-28528214, MAX.chr16.3221176-3221223, MAX.chr19.11805263-11805639, MAX.chr19.22034646-22034887, MAX.chr19.30718373-30719719, MAX.chr2.173099712-173099791, MAX.chr2.66808635-66808731, MAX.chr6.38683091-38683226, MAX.chr9.79638034-79638077, MECOM, ONECUT2, PARP15, SGIP1, SIM2, SORCS3, ST6GALNAC5, ST8SIA5, TFAP2E, TLX2, TLX3, VSTM2B, WDR17, ZNF254, ZNF43, ZNF491, ZNF763, and ZNF844.

8. The method of claim 1 or claim 2, wherein the at least one DMR is from a gene selected from FAM19A2, IFFO1, ITGB4, LOC100192379, MAX.chr1.84326495-84326656, MAX.chr16.85482306-85482494, MAX.chr6.45631561-45631625, MAX.chr7.25892382-25892451, MT1IP, NCOR2, OLIG1, RAD52, SHROOM1, SLC12A8, and TBC1D10C.

9. The method of claim 1 or claim 2, wherein the at least one DMR is from a gene selected from MAX.chr19.30718373-30719719, ITGB4, MAX.chr7.25892382-25892451, RAD52, SHROOM1, SLC12A8, and TBC1D10C.

10. The method of claim 1 or claim 2, wherein the at least one DMR is from a gene selected from ALX4, Clorf114, CA8, CCNA1, CLSTN2, CR1, DAB1, DOK1, EMBP1, EPDR1, FAM19A2, FLJ43390, FMN2, GDF6, GFRA1, HOXB3, IFFO1, ITGB4, LDLRAD2, LOC100192379, LOC648809, MAPRE2, MAX.chr1.241587339-241587784, MAX.chr1.50798781-50799423, MAX.chr1.84326495-84326656, MAX.chr13.28527984-28528214, MAX.chr16.3221176-3221223, MAX.chr16.85482306-85482494, MAX.chr19.11805263-11805639, MAX.chr19.22034646-22034887, MAX.chr19.30718373-30719719, MAX.chr2.173099712-173099791, MAX.chr2.66808635-66808731, MAX.chr6.38683091-38683226, MAX.chr6.45631561-45631625, MAX.chr7.25892382-25892451, MAX.chr9.79638034-79638077, MECOM, MT1IP, NCOR2, OLIG1, ONECUT2, PARP15, RAD52, SGIP1, SHROOM1, SIM2, SLC12A8, SORCS3, ST6GALNAC5, ST8SIA5, TBC1D10C, TFAP2E, TLX2, TLX3, VSTM2B, WDR17, ZNF254, ZNF43, ZNF491, ZNF763, and ZNF844.

11. The method of claim 1 or claim 2, wherein the at least one DMR is from a gene selected from CA8, EMBP1, HOXB3, IFFO1, ITGB4, LOC100192379, LOC648809, MAX.chr1.84326495-84326656, MAX.chr16.3221176-3221223, MAX.chr16.85482306-85482494, MAX.chr19.30718373-30719719, MAX.chr9.79638034-79638077, MT1IP, ONECUT2, SHROOM1, SIM2, SLC12A8, TLX3, and ZNF763.

12. The method of claim 1 or claim 2, wherein the at least one DMR is from a gene selected from Clorf114, CA8, CCNA1, EMBP1, EPDR1, FAM19A2, FMN2, HOXB3, IFFO1, ITGB4, LDLRAD2, LOC100192379, LOC648809, MAPRE2, MAX.chr1.50798781-50799423, MAX.chr1.84326495-84326656, MAX.chr16.3221176-3221223, MAX.chr19.11805263-11805639, MAX.chr2.66808635-66808731, MAX.chr6.38683091-38683226, MAX.chr6.45631561-45631625, MAX.chr9.79638034-79638077, MECOM, MT1IP, ONECUT2, PARP15, SHROOM1, SIM2, SLC12A8, SORCS3, ST6GALNAC5, ST8SIA5, TBC1D10C, TLX3, ZNF254, ZNF491, ZNF763, and ZNF844.

13. The method of claim 1 or claim 2, wherein the at least one DMR is from a gene selected from CA8, EMBP1, HOXB3, IFFO1, ITGB4, LOC100192379, LOC648809, MAX.chr1.84326495-84326656, MAX.chr16.3221176-3221223, MAX.chr9.79638034-79638077, MT1IP, ONECUT2, SHROOM1, SIM2, SLC12A8, TLX3, and ZNF763.

14. The method of claim 1 or claim 2, wherein the at least one DMR is from a gene selected from TLX3, MAX.chr16.3221176-3221223, TBC1D10C, and SHROOM1.

15. The method of any one of claims 1 to 14, wherein the at least one DMR is associated with an area under a ROC curve (AUC) greater than or equal to 0.5, and wherein the ROC curve discriminates between a subject having or suspected of having an oropharyngeal cancer and a control DNA sample.

16. The method of any one of claims 1 to 15, wherein the at least one DMR is comprises an increased methylation percentage as compared to a control DNA sample.

17. The method of any one of claims 1 to 15, wherein the at least one DMR is comprises an increased hypermethylation ratio as compared to a control DNA sample.

18. The method of any one of claims 15 to 17, wherein the control DNA sample is from a subject that does not have an oropharyngeal cancer.

19. The method of claim 18, wherein the control DNA sample is selected from a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and a stool sample.

20. The method of claim 19, wherein the tissue sample is selected from an oropharyngeal tissue sample, a soft palate tissue sample, a throat tissue sample, a tongue tissue sample, and a tonsil tissue sample.

21. The method of any one of claims 1 to 20, wherein the subject is a human.

22. The method of any one of claims 1 to 21, wherein the biological sample is obtained from the subject, and wherein the method further comprises extracting the DNA sample from the biological sample.

23. The method of any one of claims 1 to 22, wherein the biological sample is collected with a collection device.

24. The method of any one of claims 1 to 23, wherein the reagent that modifies DNA in a methylation-specific manner is a borane reducing agent.

25. The method of any one of claims 1 to 24, wherein the reagent that modifies DNA in a methylation-specific manner comprises one or more of a methylation-sensitive restriction enzyme, a methylation-dependent restriction enzyme, and a bisulfite reagent.

26. The method of any one of claims 1 to 25, wherein determining the methylation profile of at least one DMR comprises amplifying at least a portion of the DMR using a set of primers.

27. The method of any one of claims 1 to 26, wherein determining the methylation profile of at least one DMR comprises performing at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

28. The method of any one of claims 1 to 27, wherein determining the methylation profile of at least one DMR comprises determining the presence or absence of methylation at one or more CpG sites.

29. The method of claim 28, wherein the one or more CpG sites are present in a coding region, a non-coding region, and/or a regulatory region of a gene.

30. The method of any one of claims 1 to 29, wherein determining the methylation profile of at least one DMR comprises determining a methylation frequency.

31. The method of any one of claims 1 to 30, wherein determining the methylation profile of at least one DMR comprises determining a methylation pattern.


SEQUENCE LISTING (176 sequences)

SEQ ID NO: 1 - DNA, 29 bp, synthetic construct
  source 1..29: mol_type = other DNA
cgcgaaaagattttatattggtattacgt

SEQ ID NO: 2 - DNA, 23 bp, synthetic construct
  source 1..23: mol_type = other DNA
aacccgcacccgaaactaacgac

SEQ ID NO: 3 - DNA, 25 bp, synthetic construct
  source 1..25: mol_type = other DNA
tcgtattttgtaaggtataattcgg

SEQ ID NO: 4 - DNA, 26 bp, synthetic construct
  source 1..26: mol_type = other DNA
aactctttcctatacttcacttcgta

SEQ ID NO: 5 - DNA, 25 bp, synthetic construct
  source 1..25: mol_type = other DNA
tagagttttttatgcgtagcggcgg

SEQ ID NO: 6 - DNA, 25 bp, synthetic construct
  source 1..25: mol_type = other DNA
attctctctatatccccctcgcgaa

SEQ ID NO: 7 - DNA, 31 bp, synthetic construct
  source 1..31: mol_type = other DNA
ttttaattacgagagcgataaaaatttgcgt

SEQ ID NO: 8 - DNA, 27 bp, synthetic construct
  source 1..27: mol_type = other DNA
ccgaaataaataccgaaaaaaatcgat

SEQ ID NO: 9 - DNA, 31 bp, synthetic construct
  source 1..31: mol_type = other DNA
cgggttttagcgatgtttattttagtttcgt

SEQ ID NO: 10 - DNA, 26 bp, synthetic construct
  source 1..26: mol_type = other DNA
ccttttcgttcgtataaaatttcgtt

SEQ ID NO: 11 - DNA, 28 bp, synthetic construct
  source 1..28: mol_type = other DNA
tttttcgttcgtttttttattttttcga

SEQ ID NO: 12 - DNA, 28 bp, synthetic construct
  source 1..28: mol_type = other DNA
aacgcataccatcgacttcaactacgac

SEQ ID NO: 13 - DNA, 25 bp, synthetic construct
  source 1..25: mol_type = other DNA
ggtagtgttttttcgagacgcggtc

SEQ ID NO: 14 - DNA, 20 bp, synthetic construct
  source 1..20: mol_type = other DNA
gatacgcgaacacgcacacg

SEQ ID NO: 15 - DNA, 25 bp, synthetic construct
  source 1..25: mol_type = other DNA
tgtttatggatttaggtgaggacgg

SEQ ID NO: 16 - DNA, 25 bp, synthetic construct
  source 1..25: mol_type = other DNA
aaaaaatacaacgtttaaccgcgaa

SEQ ID NO: 17 - DNA, 25 bp, synthetic construct
  source 1..25: mol_type = other DNA
tgataggatgttcgtttagtcgcgg

SEQ ID NO: 18 - DNA, 23 bp, synthetic construct
  source 1..23: mol_type = other DNA
aaaaaactacgccgatccccgaa

SEQ ID NO: 19 - DNA, 23 bp, synthetic construct
  source 1..23: mol_type = other DNA
ggtcgtttttttagcgacgcggc

SEQ ID NO: 20 - DNA, 25 bp, synthetic construct
  source 1..25: mol_type = other DNA
cgaaaaactaaacaacccaaacgat

SEQ ID NO: 21 - DNA, 26 bp, synthetic construct
  source 1..26: mol_type = other DNA
aggagtttttatttcgttagtttcgt

SEQ ID NO: 22 - DNA, 25 bp, synthetic construct
  source 1..25: mol_type = other DNA
ataaccaccacatctaattctcgtt

SEQ ID NO: 23 - DNA, 25 bp, synthetic construct
  source 1..25: mol_type = other DNA
cgcgtaggtcggattgatttttcgt

SEQ ID NO: 24 - DNA, 23 bp, synthetic construct
  source 1..23: mol_type = other DNA
ttaattaacgcccgaacacccgc

SEQ ID NO: 25 - DNA, 25 bp, synthetic construct
  source 1..25: mol_type = other DNA
gaacgcggtttttaggagatttcga

SEQ ID NO: 26 - DNA, 23 bp, synthetic construct
  source 1..23: mol_type = other DNA
catactaccctctacgccgcgaa

SEQ ID NO: 27 - DNA, 27 bp, synthetic construct
  source 1..27: mol_type = other DNA
tggcggcggaggtagttttaaagatac

SEQ ID NO: 28 - DNA, 25 bp, synthetic construct
  source 1..25: mol_type = other DNA
tcgatcccgaccgaaaactaacgtc

SEQ ID NO: 29 - DNA, 24 bp, synthetic construct
  source 1..24: mol_type = other DNA
tttttgtttttggggcgcgaaaac

SEQ ID NO: 30 - DNA, 27 bp, synthetic construct
  source 1..27: mol_type = other DNA
atacatctcctccaccaactaccccgc

SEQ ID NO: 31 - DNA, 26 bp, synthetic construct
  source 1..26: mol_type = other DNA
ttggagagtttttcgaattttttcgc

SEQ ID NO: 32 - DNA, 19 bp, synthetic construct
  source 1..19: mol_type = other DNA
gaaaaccaccctaacgccg

SEQ ID NO: 33 - DNA, 25 bp, synthetic construct
  source 1..25: mol_type = other DNA
atttggtttaaggtagaacgagcgg

SEQ ID NO: 34 - DNA, 25 bp, synthetic construct
  source 1..25: mol_type = other DNA
tatattaaaatcgctcgacgacgaa

SEQ ID NO: 35 - DNA, 25 bp, synthetic construct
  source 1..25: mol_type = other DNA
ggggttgtttggatcgatcggaatc

SEQ ID NO: 36 - DNA, 24 bp, synthetic construct
  source 1..24: mol_type = other DNA
cccgcaaaaaaaccaaaatctcgc

SEQ ID NO: 37 - DNA, 25 bp, synthetic construct
  source 1..25: mol_type = other DNA
tataggtttcgatggcggcggttac

SEQ ID NO: 38 - DNA, 27 bp, synthetic construct
  source 1..27: mol_type = other DNA
attcgaaaaaaaccgaaaaaacgcgac

SEQ ID NO: 39 - DNA, 30 bp, synthetic construct
  source 1..30: mol_type = other DNA
tcgttaggaattagtggttggatttatcgc

SEQ ID NO: 40 - DNA, 27 bp, synthetic construct
  source 1..27: mol_type = other DNA
cgttctcttattttccgaatacgcgaa

SEQ ID NO: 41 - DNA, 25 bp, synthetic construct
  source 1..25: mol_type = other DNA
tagtttataaacgcggcggaatcgg

SEQ ID NO: 42 - DNA, 27 bp, synthetic construct
  source 1..27: mol_type = other DNA
ccaacgactactaaatcaaaaaacgca

SEQ ID NO: 43 - DNA, 31 bp, synthetic construct
  source 1..31: mol_type = other DNA
cggtacgtagtaggaaaatagattaaaacgt

SEQ ID NO: 44 - DNA, 29 bp, synthetic construct
  source 1..29: mol_type = other DNA
acaactccacatttactttcacgccaacg

SEQ ID NO: 45 - DNA, 25 bp, synthetic construct
  source 1..25: mol_type = other DNA
ttgcgtagatttttgtcgttttcgt

SEQ ID NO: 46 - DNA, 18 bp, synthetic construct
  source 1..18: mol_type = other DNA
cgaaacaacgcaaccgct

SEQ ID NO: 47 - DNA, 25 bp, synthetic construct
  source 1..25: mol_type = other DNA
tggaatattcggttcgaacgtacgt

SEQ ID NO: 48 - DNA, 25 bp, synthetic construct
  source 1..25: mol_type = other DNA
aacgaaaaaacgattactctacgca

SEQ ID NO: 49 - DNA, 25 bp, synthetic construct
  source 1..25: mol_type = other DNA
gttgggtttttaggggtttgaacgt

SEQ ID NO: 50 - DNA, 30 bp, synthetic construct
  source 1..30: mol_type = other DNA
aacgaaaactaaacgctaaaataaaaacga

SEQ ID NO: 51 - DNA, 29 bp, synthetic construct
  source 1..29: mol_type = other DNA
ggtttcgatttttaattttttttagtcgt

SEQ ID NO: 52 - DNA, 24 bp, synthetic construct
  source 1..24: mol_type = other DNA
cgctacccaaaaattcccttcccg

SEQ ID NO: 53 - DNA, 25 bp, synthetic construct
  source 1..25: mol_type = other DNA
gttgtggtgttgtttgcgttgtcgg

SEQ ID NO: 54 - DNA, 20 bp, synthetic construct
  source 1..20: mol_type = other DNA
tcgaccccctctctcccgac

SEQ ID NO: 55 - DNA, 25 bp, synthetic construct
  source 1..25: mol_type = other DNA
ggatgtttatttttagcggtttcgt

SEQ ID NO: 56 - DNA, 25 bp, synthetic construct
  source 1..25: mol_type = other DNA
acaactcaacccaaaaccgcaaacg

SEQ ID NO: 57 - DNA, 23 bp, synthetic construct
  source 1..23: mol_type = other DNA
agttcggcgttattgtttttcgt

SEQ ID NO: 58 - DNA, 20 bp, synthetic construct
  source 1..20: mol_type = other DNA
ctacaaaccctccccacgct

SEQ ID NO: 59 - DNA, 26 bp, synthetic construct
  source 1..26: mol_type = other DNA
cgtatattcgggaacgttagtagcgg

SEQ ID NO: 60 - DNA, 23 bp, synthetic construct
  source 1..23: mol_type = other DNA
gcgaacctacgcgcgaaaaaacg

SEQ ID NO: 61 - DNA, 25 bp, synthetic construct
  source 1..25: mol_type = other DNA
gtagttagtgtcgcgggtaggacgt

SEQ ID NO: 62 - DNA, 25 bp, synthetic construct
  source 1..25: mol_type = other DNA
acattccccaaccctatcgaacgtt

SEQ ID NO: 63 - DNA, 25 bp, synthetic construct
  source 1..25: mol_type = other DNA
tcggtatagtgcggaaagagtcggc

SEQ ID NO: 64 - DNA, 25 bp, synthetic construct
  source 1..25: mol_type = other DNA
acttccaaaaccgaacgaaatcgaa

SEQ ID NO: 65 - DNA, 28 bp, synthetic construct
  source 1..28: mol_type = other DNA
cggttcgtaaaagttgtaggagtatcgt

SEQ ID NO: 66 - DNA, 25 bp, synthetic construct
  source 1..25: mol_type = other DNA
aaaacattctaaaaaaaacttcgcc

SEQ ID NO: 67 - DNA, 25 bp, synthetic construct
  source 1..25: mol_type = other DNA
tttcgagttgtggaggggcgttatc

SEQ ID NO: 68 - DNA, 24 bp, synthetic construct
  source 1..24: mol_type = other DNA
aaaaaacgaaaacctaaacccgac

SEQ ID NO: 69 - DNA, 26 bp, synthetic construct
  source 1..26: mol_type = other DNA
ggggcgttatcgttttttagtaacgt

SEQ ID NO: 70 - DNA, 25 bp, synthetic construct
  source 1..25: mol_type = other DNA
attcaataatcccgaaatccccgaa

SEQ ID NO: 71 - DNA, 28 bp, synthetic construct
  source 1..28: mol_type = other DNA
tagggagtttttcgggagagttaggcgg

SEQ ID NO: 72 - DNA, 20 bp, synthetic construct
  source 1..20: mol_type = other DNA
aacactcgaataccgcgcga

SEQ ID NO: 73 - DNA, 25 bp, synthetic construct
  source 1..25: mol_type = other DNA
ttcggttgattattttatttagcgt

SEQ ID NO: 74 - DNA, 24 bp, synthetic construct
  source 1..24: mol_type = other DNA
gcgctaactattctcgattacgcc

SEQ ID NO: 75 - DNA, 25 bp, synthetic construct
  source 1..25: mol_type = other DNA
tagttgttcggggatcgttgtacgt

SEQ ID NO: 76 - DNA, 26 bp, synthetic construct
  source 1..26: mol_type = other DNA
cgaaataccgaaataaaccaaaccga

SEQ ID NO: 77 - DNA, 25 bp, synthetic construct
  source 1..25: mol_type = other DNA
tttttcgggattagagagagagcga

SEQ ID NO: 78 - DNA, 28 bp, synthetic construct
  source 1..28: mol_type = other DNA
caaaaatataccgaaaaataacgtcgtt

SEQ ID NO: 79 - DNA, 25 bp, synthetic construct
  source 1..25: mol_type = other DNA
atgataggttttaattggaggtcgg

SEQ ID NO: 80 - DNA, 23 bp, synthetic construct
  source 1..23: mol_type = other DNA
ccgaacgcgtcgctactttcgac

SEQ ID NO: 81 - DNA, 26 bp, synthetic construct
  source 1..26: mol_type = other DNA
ttttaatatatatttcgggatttcgg

SEQ ID NO: 82 - DNA, 23 bp, synthetic construct
  source 1..23: mol_type = other DNA
ccttcgcgatcccttactacgaa

SEQ ID NO: 83 - DNA, 25 bp, synthetic construct
  source 1..25: mol_type = other DNA
taggttgtagattaggaagaagcgg

SEQ ID NO: 84 - DNA, 25 bp, synthetic construct
  source 1..25: mol_type = other DNA
ttaatctcccgctacaaatcccgct

SEQ ID NO: 85 - DNA, 26 bp, synthetic construct
  source 1..26: mol_type = other DNA
cgggggaggtaaagtacggatttcgg

SEQ ID NO: 86 - DNA, 29 bp, synthetic construct
  source 1..29: mol_type = other DNA
aacccgaaaatatataataaaacgccgaa

SEQ ID NO: 87 - DNA, 26 bp, synthetic construct
  source 1..26: mol_type = other DNA
cgggggaggtaaagtacggatttcgg

SEQ ID NO: 88 - DNA, 25 bp, synthetic construct
  source 1..25: mol_type = other DNA
tcccgaattcgttataatccccgcc

SEQ ID NO: 89 - DNA, 22 bp, synthetic construct
  source 1..22: mol_type = other DNA
gttttcgggatttaggagtcgg

SEQ ID NO: 90 - DNA, 25 bp, synthetic construct
  source 1..25: mol_type = other DNA
caacctaaacgcaaaataccccgaa

SEQ ID NO: 91 - DNA, 25 bp, synthetic construct
  source 1..25: mol_type = other DNA
ggtttgggattaggagttggggtcg

SEQ ID NO: 92 - DNA, 25 bp, synthetic construct
  source 1..25: mol_type = other DNA
aataccgaaacgacaaaaaaccgaa

SEQ ID NO: 93 - DNA, 24 bp, synthetic construct
  source 1..24: mol_type = other DNA
ttaggggattttagcgggagtcgg

SEQ ID NO: 94 - DNA, 25 bp, synthetic construct
  source 1..25: mol_type = other DNA
ctaaacgaacaaacgaaaaaaacga

SEQ ID NO: 95 - DNA, 27 bp, synthetic construct
  source 1..27: mol_type = other DNA
cgggaaaggggttcggattttatacgt

SEQ ID NO: 96 - DNA, 26 bp, synthetic construct
  source 1..26: mol_type = other DNA
caaaacgacttccgaaatataacgaa

SEQ ID NO: 97 - DNA, 25 bp, synthetic construct
  source 1..25: mol_type = other DNA
tttggtgtcgcgtttttattagttc

SEQ ID NO: 98 - DNA, 25 bp, synthetic construct
  source 1..25: mol_type = other DNA
aactataaatcccccaatacccgca

SEQ ID NO: 99 - DNA, 25 bp, synthetic construct
  source 1..25: mol_type = other DNA
cggggtattttttagttttttttgc

SEQ ID NO: 100 - DNA, 25 bp, synthetic construct
  source 1..25: mol_type = other DNA
ccctacttcttaatatcccgacgtc

SEQ ID NO: 101 - DNA, 23 bp, synthetic construct
  source 1..23: mol_type = other DNA
cgtggtcgggagtttacgatcgc

SEQ ID NO: 102 - DNA, 25 bp, synthetic construct
  source 1..25: mol_type = other DNA
acgcaaccgataattaatatccgct

SEQ ID NO: 103 - DNA, 25 bp, synthetic construct
  source 1..25: mol_type = other DNA
attcggggtaggaaattattaacgt

SEQ ID NO: 104 - DNA, 23 bp, synthetic construct
  source 1..23: mol_type = other DNA
aatctttaacccctcccgccgaa

SEQ ID NO: 105 - DNA, 24 bp, synthetic construct
  source 1..24: mol_type = other DNA
ggttcgtagtcgtcgcgaagggac

SEQ ID NO: 106 - DNA, 29 bp, synthetic construct
  source 1..29: mol_type = other DNA
acaactccacatttactttcacgccaacg

SEQ ID NO: 107 - DNA, 25 bp, synthetic construct
  source 1..25: mol_type = other DNA
cgataaagttatcggggattttcga

SEQ ID NO: 108 - DNA, 18 bp, synthetic construct
  source 1..18: mol_type = other DNA
cgaaacaacgcaaccgct

SEQ ID NO: 109 - DNA, 29 bp, synthetic construct
  source 1..29: mol_type = other DNA
tttcgtttttattttagtagaaaatacgg

SEQ ID NO: 110 - DNA, 25 bp, synthetic construct
  source 1..25: mol_type = other DNA
aacgaaaaaacgattactctacgca

SEQ ID NO: 111 - DNA, 30 bp, synthetic construct
  source 1..30: mol_type = other DNA
tgatggtgaaatagtaaatttacgtacgga

SEQ ID NO: 112 - DNA, 30 bp, synthetic construct; identical to SEQ ID NO: 50
  source 1..30: mol_type = other DNA

SEQ ID NO: 113 - DNA, 25 bp, synthetic construct
  source 1..25: mol_type = other DNA
ttaacggtagtatcggtagtatcga

SEQ ID NO: 114 - DNA, 24 bp, synthetic construct
  source 1..24: mol_type = other DNA
cgctacccaaaaattcccttcccg

SEQ ID NO: 115 - DNA, 28 bp, synthetic construct
  source 1..28: mol_type = other DNA
ggttatttgttgttcgttttgcggggac

SEQ ID NO: 116 - DNA, 20 bp, synthetic construct
  source 1..20: mol_type = other DNA
tcgaccccctctctcccgac

SEQ ID NO: 117 - DNA, 28 bp, synthetic construct
  source 1..28: mol_type = other DNA
tttcgattatttagtttattgaggtcgg

SEQ ID NO: 118 - DNA, 25 bp, synthetic construct
  source 1..25: mol_type = other DNA
acaactcaacccaaaaccgcaaacg

SEQ ID NO: 119 - DNA, 25 bp, synthetic construct
  source 1..25: mol_type = other DNA
tcgggtttgttttacgttgaaacgt

SEQ ID NO: 120 - DNA, 20 bp, synthetic construct
  source 1..20: mol_type = other DNA
ctacaaaccctccccacgct

SEQ ID NO: 121 - DNA, 23 bp, synthetic construct
  source 1..23: mol_type = other DNA
cgggaaaacgtcgtagtcggcgt

SEQ ID NO: 122 - DNA, 23 bp, synthetic construct
  source 1..23: mol_type = other DNA
gcgaacctacgcgcgaaaaaacg

SEQ ID NO: 123 - DNA, 25 bp, synthetic construct
  source 1..25: mol_type = other DNA
gaaagtcggcggttaagttgttcga

SEQ ID NO: 124 - DNA, 25 bp, synthetic construct
  source 1..25: mol_type = other DNA
acattccccaaccctatcgaacgtt

SEQ ID NO: 125 - DNA, 25 bp, synthetic construct
  source 1..25: mol_type = other DNA
cgttcgtttttagcggaggtaacgc

SEQ ID NO: 126 - DNA, 25 bp, synthetic construct
  source 1..25: mol_type = other DNA
acttccaaaaccgaacgaaatcgaa

SEQ ID NO: 127 - DNA, 30 bp, synthetic construct
  source 1..30: mol_type = other DNA
agagtattttttttatttatttttacgcgg

SEQ ID NO: 128 - DNA, 25 bp, synthetic construct
  source 1..25: mol_type = other DNA
aaaacattctaaaaaaaacttcgcc

SEQ ID NO: 129 - DNA, 26 bp, synthetic construct
  source 1..26: mol_type = other DNA
cgaaatttggttggtttgttggggtc

SEQ ID NO: 130 - DNA, 24 bp, synthetic construct
  source 1..24: mol_type = other DNA
aaaaaacgaaaacctaaacccgac

SEQ ID NO: 131 - DNA, 28 bp, synthetic construct
  source 1..28: mol_type = other DNA
tttaagtcgggtttaaaggggtagggcg

SEQ ID NO: 132 - DNA, 25 bp, synthetic construct
  source 1..25: mol_type = other DNA
attcaataatcccgaaatccccgaa

SEQ ID NO: 133 - DNA, 27 bp, synthetic construct
  source 1..27: mol_type = other DNA
cgatttttcgggtttttttcgtatcgt

SEQ ID NO: 134 - DNA, 20 bp, synthetic construct
  source 1..20: mol_type = other DNA
aacactcgaataccgcgcga

SEQ ID NO: 135 - DNA, 24 bp, synthetic construct
  source 1..24: mol_type = other DNA
gaagagcgtcgggaggtttttcgc

SEQ ID NO: 136 - DNA, 24 bp, synthetic construct
  source 1..24: mol_type = other DNA
gcgctaactattctcgattacgcc

SEQ ID NO: 137 - DNA, 26 bp, synthetic construct
  source 1..26: mol_type = other DNA
cgtcgcgtatatttagtcgttatcgg

SEQ ID NO: 138 - DNA, 26 bp, synthetic construct
  source 1..26: mol_type = other DNA
cgaaataccgaaataaaccaaaccga

SEQ ID NO: 139 - DNA, 22 bp, synthetic construct
  source 1..22: mol_type = other DNA
tcgtttgtagttcggacggcga

SEQ ID NO: 140 - DNA, 28 bp, synthetic construct
  source 1..28: mol_type = other DNA
caaaaatataccgaaaaataacgtcgtt

SEQ ID NO: 141 - DNA, 25 bp, synthetic construct
  source 1..25: mol_type = other DNA
gcggttgtagttcgtagtgggtcgt

SEQ ID NO: 142 - DNA, 23 bp, synthetic construct
  source 1..23: mol_type = other DNA
ccgaacgcgtcgctactttcgac

SEQ ID NO: 143 - DNA, 20 bp, synthetic construct
  source 1..20: mol_type = other DNA
ttgggttcggttaaagtcgt

SEQ ID NO: 144 - DNA, 23 bp, synthetic construct
  source 1..23: mol_type = other DNA
ccttcgcgatcccttactacgaa

SEQ ID NO: 145 - DNA, 25 bp, synthetic construct
  source 1..25: mol_type = other DNA
ttatttttggattagcgatcgacga

SEQ ID NO: 146 - DNA, 25 bp, synthetic construct
  source 1..25: mol_type = other DNA
ttaatctcccgctacaaatcccgct

SEQ ID NO: 147 - DNA, 26 bp, synthetic construct
  source 1..26: mol_type = other DNA
taaggatgtatcgagaggtggcgtcg

SEQ ID NO: 148 - DNA, 29 bp, synthetic construct
  source 1..29: mol_type = other DNA
aacccgaaaatatataataaaacgccgaa

SEQ ID NO: 149 - DNA, 26 bp, synthetic construct
  source 1..26: mol_type = other DNA
cggaatgtcggagtaggttagatcgg

SEQ ID NO: 150 - DNA, 25 bp, synthetic construct
  source 1..25: mol_type = other DNA
tcccgaattcgttataatccccgcc

SEQ ID NO: 151 - DNA, 25 bp, synthetic construct
  source 1..25: mol_type = other DNA
tttttatcgagagatttgggcggcg

SEQ ID NO: 152 - DNA, 25 bp, synthetic construct
  source 1..25: mol_type = other DNA
caacctaaacgcaaaataccccgaa

SEQ ID NO: 153 - DNA, 23 bp, synthetic construct
  source 1..23: mol_type = other DNA
ggcgggttacgtttgattttcgg

SEQ ID NO: 154 - DNA, 25 bp, synthetic construct
  source 1..25: mol_type = other DNA
aataccgaaacgacaaaaaaccgaa

SEQ ID NO: 155 - DNA, 26 bp, synthetic construct
  source 1..26: mol_type = other DNA
aattgttttttataggtgtatagcgt

SEQ ID NO: 156 - DNA, 25 bp, synthetic construct
  source 1..25: mol_type = other DNA
ctaaacgaacaaacgaaaaaaacga

SEQ ID NO: 157 - DNA, 26 bp, synthetic construct
  source 1..26: mol_type = other DNA
attgtaggtaagcgttcgggaggggc

SEQ ID NO: 158 - DNA, 26 bp, synthetic construct
  source 1..26: mol_type = other DNA
caaaacgacttccgaaatataacgaa

SEQ ID NO: 159 - DNA, 25 bp, synthetic construct
  source 1..25: mol_type = other DNA
gggtttgtagtttatcgggcgtcgt

SEQ ID NO: 160 - DNA, 25 bp, synthetic construct
  source 1..25: mol_type = other DNA
aactataaatcccccaatacccgca

SEQ ID NO: 161 - DNA, 24 bp, synthetic construct
  source 1..24: mol_type = other DNA
ggggtcgttggaggaagggtagtc

SEQ ID NO: 162 - DNA, 25 bp, synthetic construct
  source 1..25: mol_type = other DNA
ccctacttcttaatatcccgacgtc

SEQ ID NO: 163 - DNA, 25 bp, synthetic construct
  source 1..25: mol_type = other DNA
atgtcgagtacgttagagtaggcgg

SEQ ID NO: 164 - DNA, 25 bp, synthetic construct
  source 1..25: mol_type = other DNA
acgcaaccgataattaatatccgct

SEQ ID NO: 165 - DNA, 25 bp, synthetic construct
  source 1..25: mol_type = other DNA
tgcgaggcgagtattattaacgcgc

SEQ ID NO: 166 - DNA, 23 bp, synthetic construct
  source 1..23: mol_type = other DNA
aatctttaacccctcccgccgaa

SEQ ID NO: 167 - DNA, 25 bp, synthetic construct
  source 1..25: mol_type = other DNA
agatttgggatttcggcgatagcga

SEQ ID NO: 168 - DNA, 29 bp, synthetic construct
  source 1..29: mol_type = other DNA
acaactccacatttactttcacgccaacg

SEQ ID NO: 169 - DNA, 25 bp, synthetic construct
  source 1..25: mol_type = other DNA
ttggagttttgcgtttcgttttcgt

SEQ ID NO: 170 - DNA, 18 bp, synthetic construct
  source 1..18: mol_type = other DNA
cgaaacaacgcaaccgct

SEQ ID NO: 171 - DNA, 25 bp, synthetic construct
  source 1..25: mol_type = other DNA
gaggttttaggtgttttcgttacgg

SEQ ID NO: 172 - DNA, 25 bp, synthetic construct
  source 1..25: mol_type = other DNA
aacgaaaaaacgattactctacgca

SEQ ID NO: 173 - DNA, 25 bp, synthetic construct
  source 1..25: mol_type = other DNA
gggtatagcgtaggagatagagcga

SEQ ID NO: 174 - DNA, 30 bp, synthetic construct; identical to SEQ ID NO: 50
  source 1..30: mol_type = other DNA

SEQ ID NO: 175 - DNA, 27 bp, synthetic construct
  source 1..27: mol_type = other DNA
cggttttttttagagtaggaaaaacgg

SEQ ID NO: 176 - DNA, 24 bp, synthetic construct
  source 1..24: mol_type = other DNA
cgctacccaaaaattcccttcccg